(19) United States
(12) Patent Application Publication
(10) Pub. No.: US 2013/0149311 A1
(43) Pub. Date: Jun. 13, 2013

(54) GFI1B MODULATION AND USES THEREOF

(71) Applicants: Tarik Moroy, Beaconsfield (CA); Cyrus Khandanpour, Montreal (CA); Lothar Vassen, Stolberg (DE); Ehssan Sharif-Askari, Montreal (CA)

(72) Inventors: Tarik Moroy, Beaconsfield (CA); Cyrus Khandanpour, Montreal (CA); Lothar Vassen, Stolberg (DE); Ehssan Sharif-Askari, Montreal (CA)

(73) Assignee: INSTITUT DE RECHERCHES CLINIQUES DE MONTREAL, Montreal, QC (CA)

(21) Appl. No.: 13/696,668

(22) PCT Filed: May 6, 2011
(86) PCT No.: PCT/CA11/50280

§ 371 Date: Feb. 26, 2013

Related U.S. Application Data
(60) Provisional application No. 61/332,311, filed on May 7, 2010.

(51) Int. Cl. A61K 39/395; A61K 31/454; A61K 38/10; A61K 31/7088

(52) U.S. Cl. CPC A61K 39/3955; A61K 31/7088; A61K 31/454; A61K 38/10
USPC 424/139.1; 435/375; 514/44 A; 514/322; 436/501; 506/9; 514/21.4

(57) ABSTRACT

Methods, uses and kits for increasing the number of hematopoietic stem cells (HSCs) in a biological system, such as for increasing the number of HSCs in the bone marrow and/or blood of a subject, based on the modulation of growth factor independence 1b (Gfi1b), are disclosed.

### CROSS REFERENCE TO RELATED APPLICATIONS

This application claims the benefit of U.S. provisional application Ser. No. 61/332,311, filed on May 7, 2010, which is incorporated herein by reference in its entirety.

### TECHNICAL FIELD

The present invention generally relates to hematopoietic stem cells (HSCs), and more particularly to the expansion of HSCs and their mobilization into the bloodstream, and uses thereof.

### BACKGROUND ART

Hematopoietic stem cells (HSCs) are capable of generating all lineages of blood and immune cells throughout life due to their capacity to self-renew and to differentiate into descendant blood and immune cells.

Murine hematopoietic stem cells (HSCs) are highly enriched in a bone marrow fraction defined by a combination of markers (Lin⁻, Sca-1⁺, c-kit⁺, (LSK), CD150⁺, CD48⁻) (Kiel M J et al., Cell. 2005; 121:1109-1121) and are either in a quiescent (dormant) state or undergo cell cycling (Wilson A et al. Cell. 2008. 135:1118-1129; Foudi A et al. Nat Biotechnol. 2009, 27:84-90). During cell division, one daughter cell retains its stem cell properties, whereas the other daughter cell remains a stem cell or differentiates into multipotential progenitors (MPPs; LSK, CD150⁺, CD48⁺ or CD150⁻, CD48⁺), which in turn develop into myeloid, lymphoid and erythroid effector cells. These differentiation processes are controlled by several mechanisms, among which the regulation of transcription figures very prominently.

Donor matched transplantation of bone marrow or hematopoietic stem cells (HSCs) is widely used to treat haematological malignancies and bone marrow dysfunction, but is associated with high mortality. Peripheral blood stem cells are a common source of stem cells for allogeneic hematopoietic stem cell transplantation (HSCT). They are typically collected from the blood through apheresis (or leukapheresis). The success of this type of transplantation depends on the ability of transplanted HSCs to home to the bone marrow and to expand/differentiate to repopulate the blood cell population. Thus, methods for expansion of HSC numbers and their mobilisation into the bloodstream of a donor and/or a recipient could significantly improve therapy. Currently, the peripheral stem cell yield is boosted with administration of Granulocyte-colony stimulating factor (G-CSF) to the donor, which mobilizes stem cells from the donor's bone marrow into the peripheral circulation. However, administration of G-CSF is associated with adverse effects such as mild-to-moderate bone pain after repeated administration, local skin reactions at the site of injection, splenic rupture, adult respiratory distress syndrome (ARDS), alveolar hemorrhage, hemoptysis and allergic reactions.

There is thus a need for novel strategies for increasing the expansion of HSC numbers and their mobilisation into the bloodstream of a donor and/or a recipient.

The present description refers to a number of documents, the content of which is herein incorporated by reference in their entirety.

### SUMMARY OF THE INVENTION

In a first aspect, the present invention provides a method of increasing the number of hematopoietic stem cells (HSCs) in a biological system, said method comprising contacting HSCs from said biological system with an inhibitor of growth factor independence 1b (Gfi1b).

In another aspect, the present invention provides a method of increasing the number of HSCs in the bone marrow and/or blood of a subject, said method comprising administering to said subject an effective amount of an inhibitor of Gfi1b.

In another aspect, the present invention provides a method of increasing the repopulation of HSCs in an HSC transplant recipient, said method comprising contacting the transplanted HSCs with an inhibitor of Gfi1b.

In another aspect, the present invention provides a use of an inhibitor of Gfi1b for increasing the number of hematopoietic stem cells (HSCs) in a biological system.

In another aspect, the present invention provides a use of an inhibitor of Gfi1b for the preparation of a medicament for increasing the number of hematopoietic stem cells (HSCs) in a biological system.

In another aspect, the present invention provides a use of an inhibitor of Gfi1b for increasing the number of hematopoietic stem cells (HSCs) in the bone marrow and/or blood of a subject.

In another aspect, the present invention provides a use of an inhibitor of Gfi1b for the preparation of a medicament for increasing the number of hematopoietic stem cells (HSCs) in the bone marrow and/or peripheral blood of a subject.

In another aspect, the present invention provides a use of an inhibitor of Gfi1b for increasing the repopulation of HSCs in an HSC transplant recipient.

In another aspect, the present invention provides a use of an inhibitor of Gfi1b for the preparation of a medicament for increasing the repopulation of HSCs in an HSC transplant recipient.

In another aspect, the present invention provides an inhibitor of Gfi1b for use in increasing the number of hematopoietic stem cells (HSCs) in a biological system.

In another aspect, the present invention provides an inhibitor of Gfi1b for use in the preparation of a medicament for increasing the number of hematopoietic stem cells (HSCs) in a biological system.

In another aspect, the present invention provides an inhibitor of Gfi1b for use in increasing the number of hematopoietic stem cells (HSCs) in the bone marrow and/or blood of a subject.

In another aspect, the present invention provides an inhibitor of Gfi1b for use in the preparation of a medicament for increasing the number of hematopoietic stem cells (HSCs) in the bone marrow and/or blood of a subject.

In another aspect, the present invention provides an inhibitor of Gfi1b for use in increasing the repopulation of HSCs in an HSC transplant recipient.

In another aspect, the present invention provides an inhibitor of Gfi1b for use in the preparation of a medicament for increasing the repopulation of HSCs in an HSC transplant recipient.

In another aspect, the present invention provides a composition comprising the above-mentioned inhibitor of Gfi1b and a pharmaceutically acceptable carrier.

In an embodiment, the above-mentioned contacting occurs in a transplant donor prior to the transplantation.

In an embodiment, the above-mentioned contacting occurs in said transplant recipient after the transplantation.

In an embodiment, the above-mentioned inhibitor of Gfi1b is an inhibitory nucleic acid. In a further embodiment, the above-mentioned inhibitory nucleic acid is an antisense RNA, an antisense DNA, an siRNA or an shRNA.

In another embodiment, the above-mentioned inhibitor of Gfi1b is a zinc-finger inhibitor. In a further embodiment, the above-mentioned zinc-finger inhibitor is Hoechst33342.

In another embodiment, the above-mentioned inhibitor of Gfi1b is a peptide comprising the amino acid sequence of SEQ ID NO: 18.

In another embodiment, the above-mentioned inhibitor of Gfi1b is an antibody recognizing an epitope within the amino acid sequence of SEQ ID NO: 18.

In an embodiment, the above-mentioned method, use or inhibitor of Gfi1b further comprises modulating the expression of at least one gene depicted in Table I in HSCs.

In an embodiment, the above-mentioned modulation is an increase and said at least one gene is at least one of genes Nos. 1 to 288 depicted in Table I. In a further embodiment, the above-mentioned at least one gene is a gene encoding an adhesion molecule involved in the retention of HSCs in their endosteal niche. In a further embodiment, the above-mentioned adhesion molecule involved in the retention of HSCs in their endosteal niche is VCAM-1, CXCR4 or integrin α4.

In another embodiment, the above-mentioned modulation is a decrease and said at least one gene is at least one of genes Nos. 289 to 573 depicted in Table I. In a further embodiment, the above-mentioned at least one gene is a gene encoding an adhesion molecule involved in endothelial cell adhesion. In a further embodiment, the above-mentioned adhesion molecule involved in endothelial cell adhesion is integrin β1 or integrin β3.

In another aspect, the present invention provides a method for determining whether a test compound may be useful for (i) increasing the number of hematopoietic stem cells (HSCs) in a biological system; (ii) increasing the number of HSCs in the bone marrow and/or blood of a subject; and/or (iii) increasing the repopulation of HSCs in an HSC transplant recipient, said method comprising: (a) contacting said test compound with a Gfi1b polypeptide or a fragment thereof; (b) determining whether said test compound binds to said Gfi1b polypeptide or fragment thereof wherein the binding of said test compound to said Gfi1b polypeptide or fragment thereof is indicative that said test compound may be useful for (i) increasing the number of hematopoietic stem cells (HSCs) in a biological system; (ii) increasing the number of HSCs in the bone marrow and/or blood of a subject; and/or (iii) increasing the repopulation of HSCs in an HSC transplant recipient.

In another aspect, the present invention provides a method for determining whether a test compound may be useful for (i) increasing the number of hematopoietic stem cells (HSCs) in a biological system; (ii) increasing the number of HSCs in the bone marrow and/or blood of a subject; and/or (iii) increasing the repopulation of HSCs in an HSC transplant recipient, said method comprising: (a) contacting said test compound with a cell exhibiting Gfi1b expression or activity; (b) determining whether said test compound inhibits said Gfi1b expression or activity; wherein the inhibition of said Gfi1b expression or activity in the presence of said test compound is indicative that said test compound may be useful for (i) increasing the number of hematopoietic stem cells (HSCs) in a biological system; (ii) increasing the number of HSCs in the bone marrow and/or blood of a subject; and/or (iii) increasing the repopulation of HSCs in an HSC transplant recipient.

In another aspect, the present invention provides a method for determining whether a test compound may be useful for (i) increasing the number of hematopoietic stem cells (HSCs) in a biological system; (ii) increasing the number of HSCs in the bone marrow and/or blood of a subject; and/or (iii) increasing the repopulation of HSCs in an HSC transplant recipient, said method comprising: (a) contacting said test compound with a cell comprising a first nucleic acid comprising a transcriptional regulatory element normally associated with a Gfi1b gene, operably linked to a second nucleic acid encoding a reporter protein; (b) determining whether reporter gene expression or activity is inhibited in the presence of said test compound; wherein the inhibition of said reporter gene expression or activity in the presence of said test compound is indicative that said test compound may be useful for (i) increasing the number of hematopoietic stem cells (HSCs) in a biological system; (ii) increasing the number of HSCs in the bone marrow and/or blood of a subject; and/or (iii) increasing the repopulation of HSCs in an HSC transplant recipient.

In another aspect, the present invention provides a method for determining whether a test compound may be useful for (i) increasing the number of hematopoietic stem cells (HSCs) in a biological system; (ii) increasing the number of HSCs in the bone marrow and/or blood of a subject; and/or (iii) increasing the repopulation of HSCs in an HSC transplant recipient, said method comprising: (a) contacting said test compound with a cell comprising a first nucleic acid comprising a transcriptional regulatory element comprising a Gfi1b binding sequence, operably linked to a second nucleic acid encoding a reporter protein; (b) determining whether reporter gene expression or activity is increased in the presence of said test compound; wherein the increase of said reporter gene expression or activity in the presence of said test compound is indicative that said test compound may be useful for (i) increasing the number of hematopoietic stem cells (HSCs) in a biological system; (ii) increasing the number of HSCs in the bone marrow and/or blood of a subject; and/or (iii) increasing the repopulation of HSCs in an HSC transplant recipient.

In another aspect, the present invention provides a method for determining whether a test compound may be useful for (i) increasing the number of hematopoietic stem cells (HSCs) in a biological system; (ii) increasing the number of HSCs in the bone marrow and/or blood of a subject; and/or (iii) increasing the repopulation of HSCs in an HSC transplant recipient, said method comprising: (a) contacting said test compound with a nucleic acid comprising a Gfi1b binding sequence in the presence of Gfi1b; (b) determining whether said test compound inhibits the binding of Gfi1b to said nucleic acid; wherein the inhibition of the binding of Gfi1b to said nucleic acid in the presence of said test compound is indicative that said test compound may be useful for (i) increasing the number of hematopoietic stem cells (HSCs) in a biological system; (ii) increasing the number of HSCs in the bone marrow and/or of a subject; and/or (iii) increasing the repopulation of HSCs in an HSC transplant recipient.

In an embodiment, the above-mentioned Gfi1b binding sequence comprises TAAATCAC(A/T)GCA (SEQ ID NO: 19).

In an embodiment, the above-mentioned reporter protein is luciferase.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1A shows the gating scheme for HSC and MPPs. Bone marrow cells were stained for the indicated markers and were electronically gated for Lin⁻, Sca-1⁺, c-kit⁺ cells (LSK) cells. The LSK subset was further analyzed for expression of CD150 and CD48 and was subdivided in HSCs, MPP1 and MPP2 according to published procedures. Results are representative for at least three independent experiments;

FIG. 1B shows the activity of the Gfi1b promoter followed by green fluorescence in cells isolated from Gfi1b:GFP knock-in mice based on the gating scheme indicated in FIG. 1A. As additional information, the Mean Fluorescence Intensity of GFP (MFI, representing Gfi1b promoter activity) is indicated. Representative for at least three independent experiments;

FIG. 1C shows the activity of the Gfi1 promoter is followed by green fluorescence in cells isolated from Gfi1:GFP knock-in mice (dotted lines) or Gfi1b^{+/+} mice (full lines) based on the gating scheme indicated in FIG. 1A. As additional information, the Mean Fluorescence Intensity of GFP (MFI, representing Gfi1 promoter activity) is indicated. Representative for at least three independent experiments;

FIG. 1D shows a schematic representation of the murine Gfi1b locus, and the targeting strategy to generate the conditional Gfi1b mouse allele. Exons 2 (which contains the ATG start site of Gfi1b), 3 and 4 are flanked by loxP sites. Upon activation of a Cre allele, these exons are excised, thereby abrogating the expression of the Gfi1b protein;

FIG. 1E shows a Southern Blot of DNA obtained from tails of wt (lanes 1, 2), Gfi1b^{fl/+} (lanes 3, 4) or Gfi1b^{fl/fl} (lanes 5, 6) mice. DNA samples were restricted with HindIII. Using the 5′ probe depicted in FIG. 1D, correct recombination of the locus with the targeting vector is demonstrated by appearance of a 6-kb fragment, whereas the endogenous (wild-type) restriction fragment has a length of 10.5 kb;

FIG. 1F shows a polymerase chain reaction (PCR) genotyping of DNA from tail tip cells of a MxCre tg Gfi1b^{fl/fl} mouse (1) and a wt mouse (2). Mice were injected with plpC and the detection of a ko allele is the result of contaminating lymphocytes in the tail;

FIG. 1G shows a Western Blot of Abelson transformed pre B-cell lines established from bone marrow from plpC-treated Gfi1b^{fl/fl} and MxCre tg Gfi1b^{fl/fl} injected mice. Excision of the Gfi1b locus was stimulated with interferon treatment and abrogated the expression of Gfi1b protein in these cell lines. As loading control, Ponceau staining is shown;

FIG. 2A shows the course of plpC treatment of MxCre tg Gfi1b^{fl/fl} mice and gating strategy determine HSC and MPP frequencies using the indicated markers to stain bone marrow cells. Loss of Gfi1b significantly enhances the number of HSCs defined as LSK (Lin⁻, Sca-1⁺, c-kit⁺ cells), CD150⁺, CD48⁻. Results are representative for at least 3 independent experiments;

FIG. 2B shows the frequency of HSCs in the bone marrow (n=14) of wt and Gfi1b-deficient mice, as determined by flow cytometry (p≦0.001 for both) 30 days after the first plpC injection (equivalent to 21 days after the last injection);

FIG. 2C shows the frequency of CD34⁺ and CD34⁻ HSCs in the bone marrow (n=4) of wt and Gfi1b-deficient mice, as determined by flow cytometry (p≦0.01) 30 days after the first plpC injection (equivalent to 21 days after the last injection).

FIG. 2D shows the frequency of HSCs in the spleen of wt (n=3) and Gfi1b-deficient (n=5) mice, as determined by flow cytometry (P≦0.01) 30 days after the first plpC injection (equivalent to 21 days after the last injection);

FIG. 2E shows the frequency of HSCs in the peripheral blood (n=6) of wt and Gfi1b-deficient mice, as determined by flow cytometry (P≦0.01 for both) 30 days after the first plpC injection (equivalent to 21 days after the last injection);

FIG. 2F shows Gfi1b^{fl/fl} and MxCre tg Gfi1b^{fl/fl} treated with plpC. 30 days after the first plpC injection, peripheral blood cells were analyzed by an Advia™ blood analyzer. Loss of Gfi1b decreases platelet numbers (n=6 for Gfi1bfl/fl and MxCre tg Gfi1b^{fl/fl}) (P≦0.01). Panel f): As in d) for leukocytes

FIG. 2G shows similar experiments as in FIG. 2F, for red blood cells;

FIG. 2H shows similar experiments as in FIG. 2F, for leukocytes;

FIG. 2I shows a genotyping of sorted HSC from plpC-injected MxCre tg Gfi1b^{fl/fl} mice. Excision of the Gfi1b allele was efficient, and nonexcised alleles are below detection limit in HSCs.

FIG. 3A shows the frequency of apoptosis of HSCs in the bone marrow (n=3) of wt and Gfi1b-deficient mice was determined by flow cytometry (p≦0.001 for both) using Annexin staining;

FIG. 3B shows mice intraperitoneally injected with BrdU 18 h before analysis. Bone marrow cells were stained for the indicated markers and for BrdU. A representative result from three independent examinations is shown. Mean values and standard deviations of the three independent experiments are depicted; p≦0.05 for difference in cell cycle progression between wt and Gfi1b-deficient HSCs;

FIG. 3C shows bone marrow cells of plpC-treated Gfi1b^{fl/fl} and MxCre tg Gfi1b^{fl/fl} mice stained with the specific antibodies to define HSCs, Hoechst 3342 and Verapamil according to manufacturer's instruction. Cells were then electronically gated to define HSCs (LSK, CD150⁺, CD48⁻) and Hoechst levels were determined. A histogram representative for three independent examinations is shown. Lower panel: quantification of three independent experiments for HSCs and different MPP fractions; p≦0.05 for difference in cell cycle progression between wt and Gfi1b-deficient HSCs. Values were obtained 30 days after the first (equivalent to 21 days after the last) plpC injection;

FIG. 3D shows a schematic outline to detect BrdU cells following published procedures. 40% of wt HSCs were qualified as “label retaining” whereas only 12% of Gfi1b^{ko/ko} HSCs still retained the label (BrdU) (n=4 for Gfi1b^{fl/fl} and n=4 for MxCre tg Gfi1b^{fl/fl}; p≦0.05);

FIG. 3E shows the detection of reactive oxygen species (ROS) in HSCs. Upper panel: A representative result from three independent experiments is shown. Lower panel: quantification of ROS levels in HSCs from animals with indicated genotypes (MFI, n=3). Values were obtained 30 days after the first (equivalent to 21 days after the last) plpC injection;

FIG. 3F shows the frequency of HSCs in the bone marrow of wt (n=7) and Gfi1b-deficient (n=6) mice, which received N-Acetylcystein (NAC) or were left untreated (n=14) for wt and Gfi1b-deficient). Frequency of HSCs was determined by flow cytometry (p≦0.01 between untreated and NAC treated Gfi1b-deficient HSCs). Values were obtained 30 days after the first (equivalent to 21 days after the last) plpC injection;

FIG. 3G shows the frequency of HSCs in the spleen of wt (n=3) and Gfi1b-deficient (n=4) mice, which received N-Acetylcystein or were left untreated (n=3 for wt and n=5 Gfi1b-deficient) was determined by flow cytometry (p≦0.01 between untreated and NAC-treated Gfi1b-deficient HSCs);

FIG. 3H shows the frequency of HSCs in the peripheral blood of wt (n=3) and Gfi1b-deficient (n=5) mice, which received NAC or were left untreated (n=6 for both genotypes) was determined by flow cytometry (p≦0.01 between untreated and NAC-treated Gfi1b-deficient HSCs);

FIG. 3I shows the genotyping of Gfi1b-deficient HSCs sorted from NAC- and plpC-treated Gfi1b-deficient mice. HSCs: genotyping of HSCs after treatment with NAC. NAC treatment did not affect excision of floxed Gfi1b exons and non-excised HCSs were below detection level. CTL: Two controls with one sample consisting of cells with a flox/wt constellation and one sample consisting of wt cells.

FIG. 4A shows 20,000 bone marrow cells of plpC-treated Gfi1b^{fl/fl} and MxCre tg Gfi1b^{fl/fl} mice seeded on methylcellulose. After the indicated time periods (10 days), the number of colonies was determined, cells were resuspended and 10,000 cells of the suspension were replated (n=6). Cell numbers were analyzed at indicated time points.

FIG. 4B shows a scheme depicting the transplantation of equal number of bone marrow cells. 200 000 bone marrow cells from plpC-treated Gfi1b^{fl/fl} or MxCre tg Gfi1b (Gfi1b^{ko/ko}) (both CD45.2⁺) mice were transplanted with 200 000 CD45.1⁺ bone marrow cells into lethally irradiated CD45.1⁺ mice.

FIG. 4C shows the percentage of CD45.2 positive cells (% CD45.2) in the blood after transplantation acquired at indicated time points (n=4);

FIG. 4D shows CD45 chimerism in the blood determined 24 weeks after transplantation in recipient mice (n=4) overall (All) and for the indicated lineages. Myeloid (Mac-1), B-lymphoid (B220), T-lymphoid (CD3). The difference is significant (p≦0.05) for CD45 chimerism between wt and Gfi1b-deficient cells, when all leukocytes are taken into account (All);

FIG. 4E shows CD45 chimerism determined 24 weeks after transplantation in the blood, bone marrow, spleen and thymus of recipient mice (n=4);

FIG. 4F shows the frequencies of HSCs determined in mice 24 weeks after transplantation with wt CD45.1 cells and with either wt CD45.2 BM cells or with Gfi1b-deficient CD45.2⁺ bone marrow cells (n=4);

FIG. 4G shows the relative proportion of HSCs originating from CD45.2 wt or CD45.2 Gfi1b-deficient HSCs after electronic gating on CD150⁺CD48⁻ cells depicted in FIG. 4F;

FIG. 4H shows HSCs, bone marrow (BM), splenocytes (SP), thymocytes (thy) from mice transplanted with wt CD45.1 and Gfi1b-deficient CD45.2 bone marrow cells genotyped and tested for the presence of the wt (CD45.1) and Gfi1b flox and Gfi1b ko alleles;

FIG. 4I shows the frequencies of HSCs in mice either transplanted with wt CD45.1 and wt CD45.2 bone marrow cells or mice transplanted with wt CD45.1 and Gfi1b-deficient (MxCre tg Gfi1b^{fl/fl}) bone marrow cells (n=4, p≦0.01);

FIG. 4J shows the quantification of which proportion of HSCs originates from CD45.2 wt or CD45.2 Gfi1b-deficient HSCs in mice transplanted with wt CD45.1 and wt CD45.2 bone marrow cells or mice transplanted with wt CD45.1 and Gfi1b-deficient (MxCre tg Gfi1b^{fl/fl}) (n=4, p≦0.01);

FIG. 4K shows the frequency of HSCs circulating in the peripheral blood of mice either transplanted with wt CD45.1 and wt CD45.2 bone marrow cells or mice transplanted with wt CD45.1 and CD45.2 Gfi1b-deficient (MxCre tg Gfi1b^{fl/fl}) (n=4, p≦0.01);

FIG. 4L shows the quantification of which proportion of HSCs circulating in blood originates from CD45.2 wt or CD 45.2 Gfi1b deficient HSCs in CD45.1 mice transplanted with wt CD45.1 and wt CD45.2 bone marrow cells or CD45.1 mice transplanted with wt CD45.1 and Gfi1b-deficient bone marrow cells (n=4, p≦0.01);

FIG. 4M shows the quantification of which proportion of Lin⁻, Sca-1⁺, c-kit⁺ (LSK) cells in bone marrow originate from CD45.2 wt or CD45.2 Gfi1b-deficient HSCs in CD45.1 mice transplanted with wt CD45.1 and wt CD45.2 HSCs or CD45.1 mice transplanted with wt CD45.1 and Gfi1b-deficient bone marrow cells (n=4, p≦0.01);

FIG. 5A shows 50 HSCs originating from either wt (CD45.1) or Gfi1b^{ko/ko} (CD45.2) mice transplanted into lethally irradiated CD45.1⁺ mice. 24 weeks after transplantation, mice were euthanized and examined for the contribution of Gfi1b deficient HSCs to the different lineages;

FIG. 5B shows the percentage of CD45.2 positive cells (% CD45.2) in the blood at indicated time points after transplantation (n=3);

FIG. 5C shows CD45 chimerism in the blood determined 24 weeks after transplantation in recipient mice (n=3) overall (All) and for the indicated lineages. Myeloid (Mac-1), B-lymphoid (B220), T-lymphoid (CD3). The difference is significant (p≦0.05) for CD45 chimerism between wt and Gfi1b deficient cells, when all leukocytes are taken into account (All);

FIG. 5D shows CD45 chimerism in the blood determined 24 weeks after transplantation in the blood, bone marrow, spleen and thymus of recipient mice (n=3);

FIG. 5E shows the frequency of bone marrow HSCs in mice either transplanted with wt CD45.1 and wt CD45.2 HSCs (white) or mice transplanted with wt CD45.1 and Gfi1b-deficient (MxCre tg Gfi1b^{fl/fl}) HSCs (black) was determined (n=3, p≦0.01);

FIG. 5F shows the quantification of which proportion of HSCs originates from CD45.2 wt or CD45.2 Gfi1b-deficient HSCs in mice transplanted with either wt CD45.1 and wt CD45.2 HSCs or mice transplanted with sorted HSCs cells from wt CD45.1 and Gfi1b-deficient CD45.2 mice (MxCre tg Gfi1b^{fl/fl} (n=3, p≦0.01);

FIG. 5G shows the number of HSCs circulating in the peripheral blood of mice either transplanted with wt CD45.1 and wt CD45.2 HSCs or mice transplanted with wt CD45.1 and CD45.2 Gfi1b-deficient HSCs (n=3, p≦0.01);

FIG. 5H shows the quantification of which proportion of HSCs circulating in blood originates from CD45.2 wt or CD45.2 Gfi1b-deficient HSCs in CD45.1 mice transplanted with wt CD45.1 and wt CD45.2 HSCs or CD45.1 mice transplanted with wt CD45.1 and Gfi1b-deficient HSCs (n=3, p≦0.01);

FIG. 5I shows the quantification of which proportion of Lin⁻, Sca-1⁺, c-kit⁺ (LSK) cells in bone marrow originate from CD45.2 wt or CD 45.2 Gfi1b-deficient HSCs in mice transplanted with wt CD45.1 and wt CD45.2 HSCs or CD45.1 mice transplanted with wt CD45.1 and Gfi1b-deficient HSCs (n=3, p≦0.01);

FIG. 5J shows the results of a serial transplantation experiment. Mice were transplanted with bone marrow from wt CD45.1 and Gfi1b-deficient (CD45.2) mice. After 24 weeks, chimerism in peripheral blood was determined and 2 Mio. bone marrow of these chimeric mice was transplanted into new lethally irradiated CD45.1 recipient mice. After 16 weeks, chimerism in the blood in these secondary transplanted mice was determined. The percentage of CD45.2 cells in the blood of the secondary transplant recipients was compared to that from the first transplant. The observed chimerism in the first transplant was set to 100%. (n=7 for second transplant, p≦0.15);

FIG. 5K shows cells from 50 μl of blood obtained from wt CD45.2 or Gfi1b-deficient CD45.2 mice and transplanted together with 200 000 bone marrow cells from wt CD45.1 mice. 12 weeks after transplantation, the number of CD45.2 cells (which was set to 1 for CD45.2 Gfi1b-deficient blood cells) within all hematopoietic cells (CD45) in blood was determined. As a control for specificity of the CD45.2 antibody, blood obtained from an untreated CD45.1 mouse was used.

FIG. 6A shows a flow cytometry analysis of bone marrow cells of plpC-treated wt, MxCre tg Gfi1b^{fl/fl}, MxCre tg Gfi1^{fl/fl} and MxCre tg Gfi1^{fl/fl} Gfi1b^{fl/fl} mice after electronic gating for LSK cells and for the indicated markers. Results for MxCre tg Gfi1^{fl/fl} Gfi1b^{fl/fl} are obtained 15 days after the first plpC injection (4 days after the last plpC injection);

FIG. 6B shows a similar analysis as FIG. 6A, with frequencies depicted in % with regard to total bone marrow (*p≦0.05; ***; p≦0.001; n=14 for wt, n=14 for MxCre tg Gfi1b^{fl/fl} and n=3 for MxCre tg Gfi1^{fl/fl});

FIG. 6C shows that the simultaneous deletion of Gfi1 and Gfi1b reduced the frequency of HSCs in bone marrow by ten-fold about 15 days after the first plpC injection of HSCs (** p≦0.01). Frequencies of HSCs reach again normal (wild type) levels in plpC injected MxCre tg Gfi1^{fl/fl} Gfi1b^{fl/fl} mice, when measured 40 days after the first plpC injection (n=14 for wt, n=14 for MxCre tg Gfi1b^{fl/fl}, n=3 for MxCre tg Gfi1^{fl/fl} and n=3 for MxCre tg Gfi1^{fl/fl} Gfi1b^{fl/fl});

FIG. 6D shows the genotyping of sorted HSCs of plpC injected MxCre tg Gfi1^{fl/fl} Gfi1b^{fl/fl} mice 15 days after the first plpC injection. Excision of the Gfi1 allele is complete, showing the presence of a functional Cre recombinase, but excision of the Gfi1b allele is incomplete.

FIG. 7A shows Gfi1^{GFP/wt} (dotted, middle line), wt (full, left line with grey area) and Gfi1b^{fl/fl} Gfi1^{GFP/wt} (dashed, right line) mice injected with plpC. 30 days after the first injection (equivalent to 21 days after the last injection) mice were sacrificed and examined for expression of GFP, which follows the activity of the Gfi1 promoter. Loss of Gfi1b leads to an enhanced activity of the Gfi1 promoter;

FIG. 7B shows a real time PCR analysis of Gfi1 gene expression in HSCs from mice with the indicated genotypes (n=3);

FIG. 7C shows an overview of genes differentially expressed in wt and Gfi1b-deficient HSCs. Light grey bars represent relatively high expression levels and dark grey bars low expression levels (average fold induction or repression) in Gfi1b^{ko/ko} HSCs compared to wt HSCs. CXCR4 (chemokine (C-X-C motif) receptor 4) and VCAM-1 (vascular cell adhesion molecule-1) were not included in the GSEA defined adhesion molecule pathway but were also down-regulated at the RNA level.

FIG. 7D shows the expression level of different surface adhesion proteins. The expression of these proteins was changed in a manner analogous to the gene expression array results. Mean Fluorescence Intensities (MFI) of the respective surface molecules in Gfi1b^{ko/ko} (ko, black line) and wt HSCs (wt, grey line) are depicted. Dotted line indicates isotype controls;

FIG. 8A shows the amino acid sequence of human Gfi1b polypeptide, isoform 1 (GenBank accession No. NP_{—}004179, SEQ ID NO:2);

FIG. 8B shows the nucleotide sequence of the transcript encoding human Gfi1b polypeptide, isoform 1 (GenBank accession No. NM_{—}004188, SEQ ID NO:1). The coding region (nucleotides 152 to 1144) is indicated in bold;

FIG. 8C shows the amino acid sequence of human Gfi1b polypeptide, isoform 2 (GenBank accession No. NP_{—}001128503, SEQ ID NO:4);

FIG. 8D shows the nucleotide sequence of the transcript encoding human Gfi1b polypeptide, isoform 2 (GenBank accession No. NM_{—}001135031, SEQ ID NO:3). The coding region (nucleotides 152 to 1006) is indicated in bold;

FIG. 8E shows the amino acid sequence of mouse Gfi1b polypeptide, isoform 1 (GenBank accession No. NP_{—}032140, SEQ ID NO:6)

FIG. 8F shows the nucleotide sequence of the transcript encoding mouse Gfi1b polypeptide, isoform 1 (GenBank accession No. NM_{—}008114, SEQ ID NO:5). The coding region (nucleotides 156 to 1148) is indicated in bold;

FIG. 8G shows the amino acid sequence of mouse Gfi1b polypeptide, isoform 2 (GenBank accession No. NP_{—}001153878, SEQ ID NO:8);

FIG. 8H shows the nucleotide sequence of the transcript encoding mouse Gfi1b polypeptide, isoform 2 (GenBank accession No. NM_{—}001160406, SEQ ID NO:7). The coding region (nucleotides 156 to 1247) is indicated in bold; and

FIGS. 9A to 9E show the nucleotide sequence of the genomic-integrated part of the Gfi1b conditional knock-out plasmid construct (SEQ ID NO:9). The sequences of the pBSII-SK+ plasmid backbone and the diphtheria toxin fragment A (DTA) selection marker are not shown, but the sequence of the PGK1-neo resistance gene is included. Introns and exons are shown in lowercase and uppercase, respectively.

### DISCLOSURE OF INVENTION

In the studies described herein, the present inventors have shown that Gfi1b-deficient mice exhibit higher numbers of HSCs in the bone marrow and in peripheral blood. They have also demonstrated that Gfi1b-deficient HSCs retain their ability to self-renew and to initiate multilineage differentiation, are less quiescent than wild-type HSCs, and that this feature is cell autonomous as they also exhibit these features in a host following transplantation. The present inventors have shown that Gfi1b deficiency is associated with a modulation in the expression of several genes, notably genes encoding surface adhesion molecules involved in HSCs homing/trafficking.

Accordingly, in a first aspect, the present invention provides a method of increasing the number of hematopoietic stem cells (HSCs) in a biological system (e.g., a subject, an organ, a tissue, a cell culture), said method comprising inhibiting growth factor independence 1b (Gfi1b) expression or activity in HSCs from said biological system, in an embodiment comprising contacting HSCs from said biological system with an inhibitor of Gfi1b.

In another aspect, the present invention provides a method of increasing the number of HSCs (e.g., by stimulating the proliferation of HSCs) in a subject (in an organ or a tissue of a subject, such as the bone marrow and/or peripheral blood), said method comprising administering to said subject an effective amount of an inhibitor of Gfi1b.

In another aspect, the present invention provides a method of increasing the repopulation of HSCs in an HSC transplant recipient, said method comprising contacting the transplanted (or to be transplanted) HSCs with an inhibitor of Gfi1b. In an embodiment, the above-mentioned contacting occurs in a transplant donor prior to the transplantation. In another embodiment, the above-mentioned contacting occurs in said transplant recipient after the transplantation. In another embodiment, the above-mentioned contacting occurs in vitro or ex vivo to increase the number of HSCs in a sample collected from a HSC donor, prior to transplantation to said recipient. In further embodiments, the above-mentioned contacting occurs at multiple times, e.g., in a transplant donor prior to the transplantation, in vitro or ex vivo in a sample obtained from a donor prior to the transplantation, and/or in the transplant recipient after the transplantation.

The present inventors have shown that Gfi1b deficiency is associated with a modulation in the expression of several genes in HSCs, and more particularly those depicted in Table 6 that show at least a two-fold difference in expression between GFi1b-deficient HSCs and wild-type HSCs. Accordingly, in an embodiment, the above-mentioned method comprises modulating the expression of at least one gene depicted in Table 6 in HSCs.

In a further embodiment, the above-mentioned modulation is an increase and said at least one gene is at least one of genes Nos. 1 to 288 depicted in Table 6. In a further embodiment, the above-mentioned at least one gene is a gene encoding an adhesion molecule involved in the retention of HSCs in their endosteal niche, such as VCAM-1, CXCR4 or integrin α4.

In another embodiment, the above-mentioned modulation is a decrease and said at least one gene is at least one of genes Nos. 289 to 573 depicted in Table 6. In a further embodiment, the above-mentioned at least one gene is a gene encoding an adhesion molecule involved in endothelial cell adhesion, such as integrin β1 or integrin β3.

The term “Hematopoietic stem cells (HSCs)” as used herein refers to multipotent stem cells that give rise to all the blood cell types from the myeloid (monocytes and macrophages, neutrophils, basophils, eosinophils, erythrocytes, megakaryocytes/platelets, dendritic cells), and lymphoid (T-cells, B-cells, NK-cells) lineages. These cells may be isolated from the blood or bone marrow, can renew itself, can differentiate to a variety of specialized cells, and/or can mobilize out of the bone marrow into circulating blood. There appear to be two major types of HSCs that differ in their self-renewal capacity, namely short-term HSCs (defined as CD34⁺ LSK, CD150⁺, CD48⁻) that have the capacity for self-renewal for a limited time prior to full differentiation into a specific lineage, and long-term (CD34⁻ LSK, CD150⁺, CD48⁻) HSCs that have the capacity for self-renewal throughout the life span of an organism.

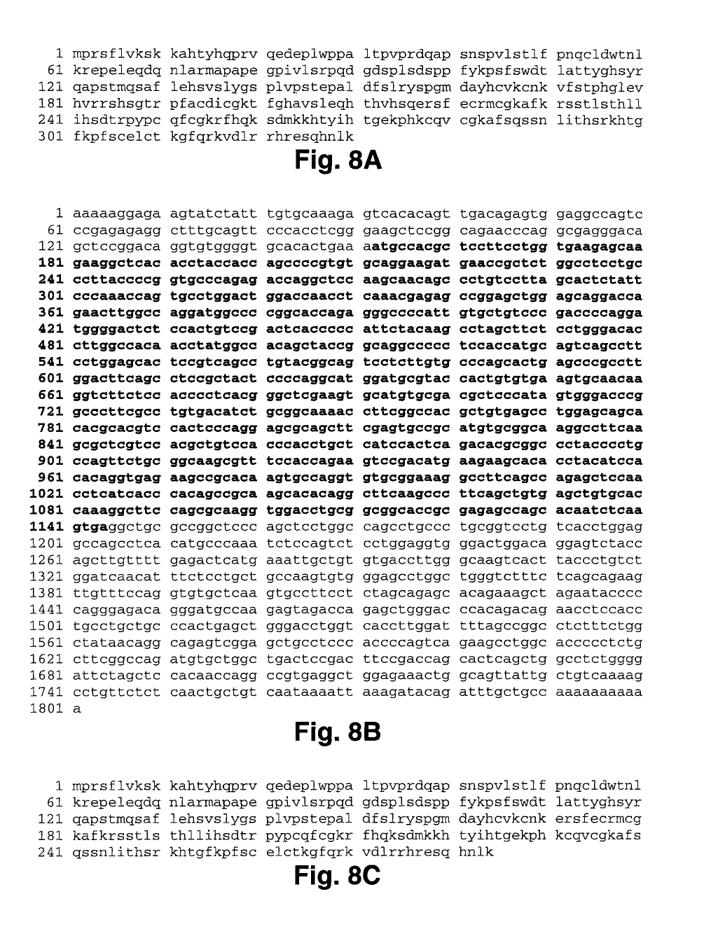

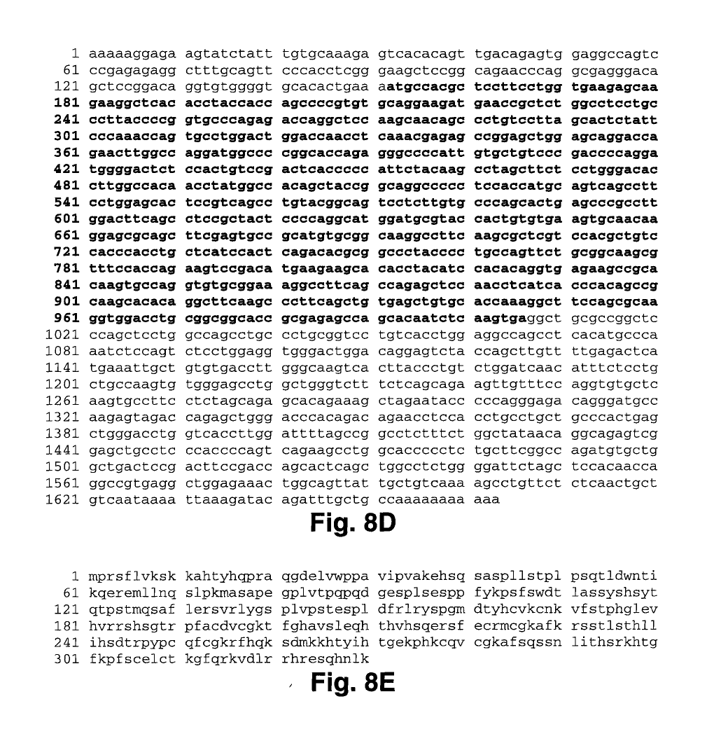

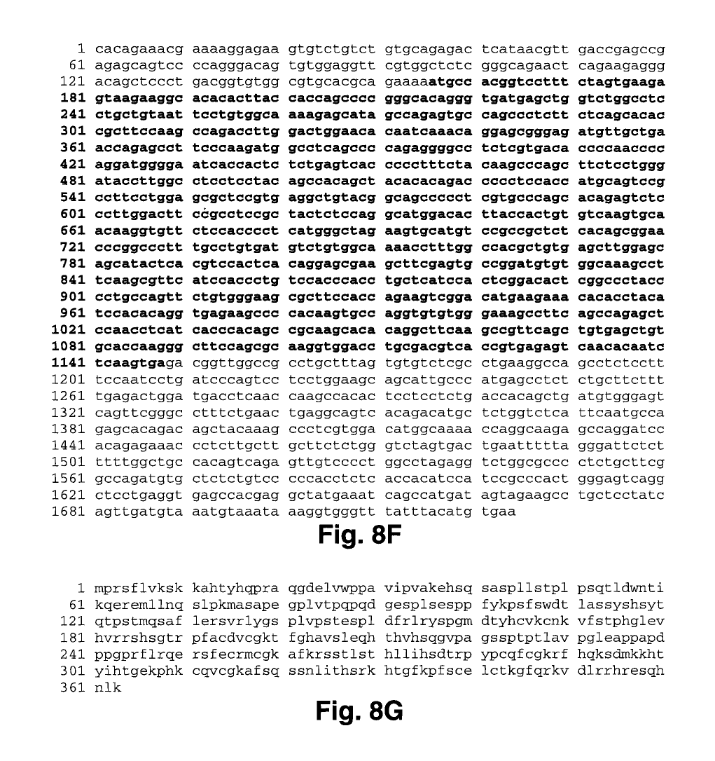

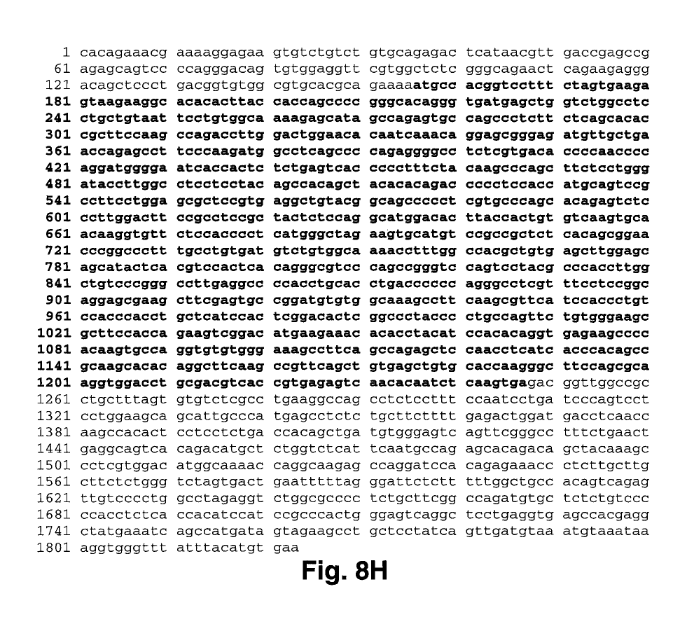

Growth factor independence-1b (Gfi1b) is a transcriptional repressor expressed in various hematopoietic cell populations, and more particularly in erythroid and megakaryocytic cells. It comprises at its N-terminus a highly conserved Snail/Gfi1 (SNAG) domain (extending from residue 1 to about residue 20) involved in transcriptional repression (notably involved in the suppression of GATA-1-mediated transcription of the Gfi-1B promoter, Huang et al., Nucleic Acids Res. 2005; 33(16): 5331-5342). The SNAG domain of Gfi1b is involved in the interaction with the chromatin regulatory proteins REST corepressor (CoREST) and lysine-specific demethylase 1 (LSD1 or KDM1), which in turn play a role in Gfi1b-mediated transcriptional repression (Saleque et al. 2007, Mol. Cell, 27(4), pp. 562-572). HDACs 1 and 2 are also part of the repression complex. Gfi1b also comprises six C2H2-type zinc finger domains (residues 163-186; 192-214; 220-242; 248-270; 276-298; and 304-327) involved in DNA binding and acting as an activation domain at its C-terminus (UniProtKB/Swiss-Prot accession No. Q5VTD9). Residues 91-330 are involved in the interaction with the E3 ubiquitin-protein ligase ARIH2, which is involved in protein ubiquitination and proteasomal degradation. Residues 164-330 are involved in the interaction with GATA-1 (Huang et al., Nucleic Acids Res. 2005; 33(16): 5331-5342). It also interacts with histone methyltransferases EHMT2 and SUV39H1, and thus alters histone methylation by recruiting them to target genes promoters. Mutation at residues 290 (Asn to Ser substitution) has been shown to prevent DNA binding (Wei X. and Kee B. L. Blood 109:4406-4414 (2007)). Two Gfi1b isoforms exist, with isoform 2 lacking residues 171-216 relative to isoform 1 (see FIGS. 8A and 8C).

As used herein, an inhibitor of Gfi1b (or Gfi1b antagonist) refers to an agent that is capable of reducing Gfi1b activity and/or its protein or nucleic acid levels (directly or indirectly), which in an embodiment includes agents that act directly on a Gfi1b protein or nucleic acid. In embodiments, such a decrease comprises a decrease Gfi1b protein activity or levels, a decrease Gfi1b mRNA levels, a decrease Gfi1b transcription or translation, or any combination thereof. General classes of inhibitors of Gfi1b include, but are not limited to, inhibitory nucleic acids, e.g., oligonucleotides containing the Gfi1b binding site, siRNA, antisense, DNAzymes, and ribozymes; small organic or inorganic molecules, e.g., zinc finger inhibitors; peptides (e.g., peptides that bind Gfi1b or to a binding partner thereof such as LSD1 and inhibit Gfi1b-mediated transcriptional repression); proteins, (e.g., dominant negatives of Gfi1b, which compete with Gfi1b for binding to its sequence on DNA but do not exert transcriptional regulation activity, or compete with Gfi1b for binding to LSD1 and/or CoREST), antibodies (antibodies that block the interaction between Gfi1b and one or more of its binding partners such as LSD1 and/or CoREST, or that block the interaction between Gfi1b and its target sequence). An inhibitor that acts directly on Gfi1b, for example, can affect binding of Gfi1b to its target nucleic acid (Wu et al., Nucleic Acids Research 35(7): 2390-2402), can sequester Gfi1b away from the nucleus (thus inhibiting its transcriptional regulation activity), can induce the degradation of Gfi1b protein or mRNA (e.g. increasing proteosomal degradation), can impair Gfi1 b transcription and/or translation.

### Inhibitors of Zinc Finger Proteins

Inhibitors of zinc finger proteins may be used to inhibit Gfi1b activity. Zinc finger inhibitors can work by, e.g., disrupting the zing finger by modification of one or more cysteine residues in the binding sites for Zn²⁺ in the zinc finger protein, resulting in the ejection of zinc ion; removing the zinc from the zinc finger moiety, e.g., by specific chelating agents, also known as “zinc ejectors”, including azodicarbonamide (ADA); or forming a ternary complex at the site of zinc binding on zinc finger proteins, resulting in inhibition of the DNA or RNA binding activity of zinc finger proteins. A number of small molecule inhibitors of zinc fingers are known in the art. For example, picolinic acid derivatives such as a small molecule called Picolinic acid drug substance (PCL-016), and a derivative thereof FSR-488, as described in U.S. Patent Publication No. 2005/0239723, and commercially available from Novactyl (St. Louis, Mo.). Other picolinic acid derivatives with zinc-binding capabilities are described in U.S. Pat. No. 6,410,570. In an embodiment, the agent is a compound that interferes with the binding of zinc-finger containing proteins to DNA, such as Hoechst33342 (Wu et al., Nucleic Acids Research 35(7): 2390-2402).

### RNA/DNA Interference

RNAi is a process whereby double-stranded RNA (dsRNA) induces the sequence-specific degradation of homologous mRNA in cells. In mammalian cells, RNAi can be triggered by duplexes of small interfering RNA (siRNA) (Chiu et al., Mol. Cell. 10:549-561 (2002); Elbashir et al., Nature 411:494-498 (2001)), or by micro-RNAs (miRNA), functional small-hairpin RNA (shRNA), or other dsRNAs which are expressed in vivo using DNA templates with RNA polymerase III promoters.

The initial agent for RNAi in some systems is thought to be dsRNA or modified dsRNA molecules corresponding to a target nucleic acid (e.g., Gfi1b). The dsRNA is then thought to be cleaved into short interfering RNAs (siRNAs) which are for example 21-23 nucleotides in length (19-21 bp duplexes, each with 2 nucleotide 3′ overhangs). The enzyme thought to effect this first cleavage step (the Drosophila version is referred to as “Dicer”) is categorized as a member of the RNase III family of dsRNA-specific ribonucleases. Alternatively, RNAi may be effected via directly introducing into the cell, or generating within the cell by introducing into the cell an siRNA or siRNA-like molecule or a suitable precursor (e.g., vector encoding precursor(s), etc.) thereof. An siRNA may then associate with other intracellular components to form an RNA-induced silencing complex (RISC). The RISC thus formed may subsequently target a transcript of interest via base-pairing interactions between its siRNA component and the target transcript by virtue of homology, resulting in the cleavage of the target transcript approximately 12 nucleotides from the 3′ end of the siRNA. Thus the target mRNA is cleaved and the level of protein product it encodes is reduced.

The nucleic acid molecules or constructs can include dsRNA molecules comprising about 16 to 30 residues, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is complementary to the first strand. The nucleic acid compositions can include both siRNA and modified siRNA derivatives, e.g., siRNAs modified to alter a property such as the pharmacokinetics of the composition, for example, to increase half-life in the body, as well as engineered RNAi precursors.

RNAi may be effected by the introduction of suitable in vitro synthesized siRNA or siRNA-like molecules into cells. RNAi may for example be performed using chemically-synthesized RNA or modified RNA molecules. Alternatively, suitable expression vectors may be used to transcribe such RNA either in vitro or in vivo. In vitro transcription of sense and antisense strands (encoded by sequences present on the same vector or on separate vectors) may be effected using for example T7 RNA polymerase, in which case the vector may comprise a suitable coding sequence operably-linked to a T7 promoter. The in vitro-transcribed RNA may in embodiments be processed (e.g., using E. coli RNase III) in vitro to a size conducive to RNAi. The sense and antisense transcripts are combined to form an RNA duplex which is introduced into a target cell of interest. Other vectors may be used, which express small hairpin RNAs (shRNAs) which can be processed into siRNA-like molecules. Various vector-based methods have been described (see, e.g., Brummelkamp et al. [2002] Science 296: 550). Various methods for introducing such vectors into cells, either in vitro or in vivo (e.g., gene therapy) are known in the art.

Reagents and kits for performing RNAi are available commercially from, for example, Ambion Inc. (Austin, Tex., USA), New England Biolabs Inc. (Beverly, Mass., USA) and Invitrogen (Carlsbad, Calif., USA).

siRNA directed against human Gfi1b are commercially available from several suppliers, including Invitrogen (Gfi1b Stealth RNAi™ siRNA, cat. #HSS188732, HSS188733 and HSS188734), Santa Cruz Biotechnology, inc. (Cat. #sc-37909), Sigma-Aldrich (MISSION° siRNA, Cat. #SASI_Hs01_{—}00223543, SASI_Hs01_{—}00223544, SASI_Hs01_{—}00223545, SASI_Hs01_{—}00223546, SASI_Hs02_{—}00337076, SASI_Hs01_{—}00223547, SASI_Hs01_{—}00223548, SASI_Hs01_{—}00223549, SASI_Hs01_{—}00223550, SASI_Hs01_{—}00223551 and SASI_Hs01_{—}00223552). ShRNA molecules targeting human Gfi1b are described, for example, in Randrianarison-Huetz et al., Blood, 2010; 115: 2784-2795 (sequences of encoding DNA: 5′-GCCTAGCTTCTCCTGGGACTTCAAGAGAGTCCCAGGAGAAGCTAG-3′, SEQ ID NO: 15; 5′-CCCATTCTACAAGCCTAGCTT-3′, SEQ ID NO: 16; and 5′-CCTTAGCACTCTATTCCCAAA-3′, SEQ ID NO: 17;) and are also commercially available from several suppliers including OriGene Technologies (Cat. #TR312792); Santa Cruz Biotechnology, inc. (Cat. #sc-37909-SH), GeneCopoeia (Cat. #HSH020142), Sigma-Aldrich, (Cat. No. SHCLNG-NM_{—}004188).

In addition, Morpholinos represent an advanced form of antisense DNA, which allows repression of a target gene (e.g., Gfi1b) expression with a greater efficiency and are commercially available (GENE TOOLS).

### Antibodies

In an embodiment, the above-mentioned Gfi1b inhibitor is a Gfi1b-specific antibody.

By Gfi1 b-specific antibody in the present context is meant an antibody capable of detecting (i.e. binding to) a Gfi1b or a Gfi1b protein fragment. In an embodiment, the above-mentioned antibody inhibits the biological activity of Gfi1b, such as Gfi1b interaction with its target sequence on DNA (e.g., by binding to one or more of its zinc finger domains). In an embodiment, the antiboby blocks the interaction between Gfi1b and one or more of its partners involved in transcriptional repression (e.g., CoREST and/or LSD1) for example by binding to an epitope located within the SNAG domain of Gfi1b (residues 1 to 20, SEQ ID NO: 18).

The term antibody or immunoglobulin is used to refer to monoclonal antibodies (including full-length monoclonal antibodies), polyclonal antibodies, multispecific antibodies, and antibody fragments so long as they exhibit the desired biological activity. Antibody fragments comprise a portion of a full length antibody, generally an antigen binding or variable region thereof. Examples of antibody fragments include Fab, Fab′, F(ab′)2, and Fv fragments, diabodies, linear antibodies, single-chain antibody molecules, single domain antibodies (e.g., from camelids), shark NAR single domain antibodies, and multispecific antibodies formed from antibody fragments. Antibody fragments can also refer to binding moieties comprising CDRs or antigen binding domains including, but not limited to, V_{H} regions (V_{H}, V_{H}-V_{H}), anticalins, PepBodies, antibody-T-cell epitope fusions (Troybodies) or Peptibodies. Additionally, any secondary antibodies, either monoclonal or polyclonal, directed to the first antibodies would also be included within the scope of this invention.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody A Laboratory Manual, CSH Laboratories).

Polyclonal antibodies are preferably raised in animals by multiple subcutaneous (s.c.), intravenous (i.v.) or intraperitoneal (i.p.) injections of the relevant antigen (e.g., Gfi1b polypeptide or a fragment thereof) with or without an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals may be immunized against the antigen (e.g., a Gfi1b polypeptide or a fragment thereof), immunogenic conjugates, or derivatives by combining the antigen or conjugate (e.g., 100 μg for rabbits or 5 μg for mice) with 3 volumes of Freund's complete adjuvant and injecting the solution intradermally at multiple sites. One month later the animals are boosted with the antigen or conjugate (e.g., with ⅕ to 1/10 of the original amount used to immunize) in Freund's complete adjuvant by subcutaneous injection at multiple sites. Seven to 14 days later the animals are bled and the serum is assayed for antibody titer. Animals are boosted until the titer plateaus. Preferably, for conjugate immunizations, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal antibodies may be made using the hybridoma method first described by Kohler et al., Nature, 256: 495 (1975), or may be made by recombinant DNA methods (e.g., U.S. Pat. No. 6,204,023). Monoclonal antibodies may also be made using the techniques described in U.S. Pat. Nos. 6,025,155 and 6,077,677 as well as U.S. Patent Application Publication Nos. 2002/0160970 and 2003/0083293.

In the hybridoma method, a mouse or other appropriate host animal, such as a rat, hamster or monkey, is immunized (e.g., as hereinabove described) to elicit lymphocytes that produce or are capable of producing antibodies that will specifically bind to the antigen used for immunization. Alternatively, lymphocytes may be immunized in vitro. Lymphocytes then are fused with myeloma cells using a suitable fusing agent, such as polyethylene glycol, to form a hybridoma cell.

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Antibodies directed against Gfi1b and which may inhibit Gfi1b activity are known in the art (see, e.g., Laurent et al., Stem Cells. 2009; 27(9):2153-2162) and are also commercially available (Abnova Corporation, Cat. #H00008328-A01; Abcam, Cat. #ab26132; Sigma-Aldrich, Cat. #HPA007012 and AV30093).

### Other Inhibitors

Gfi1b inhibitors may also be in the form of non-antibody-based scaffolds, such as avimers (Avidia); DARPins (Molecular Partners); Adnectins (Adnexus), Anticalins (Pieris) and Affibodies (Affibody). The use of alternative scaffolds for protein binding is well known in the art (see, for example, Binz and Plückthun, 2005, Curr. Opin. Biotech. 16: 1-11).

In an embodiment, the Gfi1b inhibitor is a dominant negative of Gfi1b (or a nucleic acid encoding same), for example a variant of Gfi1b (in which one or more domains are mutated or deleted, for example) which compete with Gfi1b (for binding to DNA or to one or more of its binding partner) but do not exert transcriptional regulation activity. In an embodiment, the dominant negative comprises one or more of the C2H2-type zinc finger domains but lacks a functional SNAG domain (e.g., lack residues 1 to 20 or a portion thereof), and thus competes with endogenous Gfi1b for binding to DNA but is unable to bind to its partners involved in transcriptional repression (e.g., CoREST and/or LSD1) and to exert transcriptional repression activity.

In another embodiment, the dominant negative comprises the SNAG domain of Gfi1b (residues 1 to 20, SEQ ID NO:18) but lack one or more of the C2H2-type zinc finger domains and thus competes with endogenous Gfi1b for binding to its partners involved in transcriptional repression (e.g., CoREST and/or LSD1), but cannot bind DNA.

In an embodiment, the Gfi1b inhibitor is a peptide comprising the sequence of SEQ ID NO: 18, or a fragment thereof, or a variant thereof, having Gfi1b inhibiting activity. In an embodiment, the above-mentioned peptide (or fragment/variant thereof) contains from about 10 to about 200 amino acids, e.g., from about 20 to about 200 amino acids. In a further embodiment, the above-mentioned peptide (or fragment/variant thereof) contains from about 10 to about 100 amino acids. In a further embodiment, the above-mentioned peptide (or fragment/variant thereof) contains from about 10 to about 90 amino acids. In a further embodiment, the above-mentioned peptide (or fragment/variant thereof) contains from about 10 to about 80 amino acids. In a further embodiment, the above-mentioned peptide (or fragment/variant thereof) contains from about 10 to about 70 amino acids. In a further embodiment, the above-mentioned peptide (or fragment/variant thereof) contains from about 10 to about 60 amino acids. In a further embodiment, the above-mentioned peptide (or fragment/variant thereof) contains from about 10 to about 50 amino acids. In a further embodiment, the above-mentioned peptide (or fragment/variant thereof) contains from about 10 to about 40 amino acids, e.g., from about 10 to about 30, from about 15 to about 25. In an embodiment, the peptide (or fragment/variant thereof) contains about 20 amino acids (18, 19, 20, 21 or 22 amino acids). In another embodiment, the above-mentioned fragment or variant binds to CoREST and/or LSD1. In an embodiment, the above-mentioned variant comprises a domain that is at least 75, 80, 85, 90, or 95% identical to the sequence of SEQ ID NO: 18.

In an embodiment, the Gfi1b inhibitor is a peptide consisting of the sequence of SEQ ID NO: 18.

Other reagents for inhibiting Gfi1b expression include the CompoZr™ Knockout ZFNs kit from Sigma-Aldrich (Cat. #CKOZFN9240-1KT). Such reagent creates targeted double strand breaks at the specific gene (Gfi1b) locus, and, through the cellular process of Non-Homologous End Joining (NHEJ), this double strand break can result in modification of the DNA sequence and therefor create a functional knockout of the targeted gene (Gfi1b).

Other reagents for inhibiting Gfi1b expression include agents that indirectly act on Gfi1b transcription. For example, GATA-1 is known to bind to the Gfi1b promoter and stimulate Gfi1b transcription. Therefore, the inhibitor of Gfi1b may be an agent that decrease the activity or expression of GATA-1. Similarly, Gfi1b interacts with the E3 ubiquitin-protein ligase ARIH2 (also known as TRIAD1), which is involved in protein ubiquitination and subsequent proteasomal degradation. E3 ubiquitin ligases catalyze the covalent conjugation of ubiquitin to specific substrate proteins and depending on the type/nature of the ubiquitin chain conjugated to the protein, ubiquitination can regulate its activity or stability. TRIAD1 has been shown to interact with the DNA-binding domain of Gfi1 and Gfi1b (whose zinc finger domain are 97% identical), and to inhibit Gfi1 ubiquitination, resulting in a prolonged half-life and in increased endogenous Gfi1 protein levels (Marteijn J A et al., Blood. Nov. 1, 2007; 110(9):3128-35. Epub Jul. 23, 2007). Thus, decreasing the activity or expression of ARIH2/TRIAD1 in a HSC may be used to increase ubiquitination and proteasomal degradation of Gfi1b, thus decreasing its expression/activity. In an embodiment, ARIH2 expression is decreased using a siRNA, such as those described in Marteijn J A et al., 2007, supra (uugugaggaagaggaagaa, SEQ ID NO: 13; aauugugaggaagaggaagaa, SEQ ID NO: 14). Also, siRNA directed against human HMGB2 are commercially available from Sigma-Aldrich (MISSION® siRNA, Cat. #SASI_Hs01_{—}00230799 to SASI_Hs01_{—}00230808, SASI_Hs02_{—}00341344 and SASI_Hs02_{—}00341345) and Origene (Cat. #SR307069). ShRNA directed against human ARIH2 are also commercially available from Sigma-Aldrich (MISSION® shRNA Plasmid DNA, Cat. #SHCLND-NM_{—}006321) and Origene (Cat. #TG314665).

Similarly, the high-mobility group HMG protein HMGB2 has been shown to bind to the Gfi1b promoter in vivo and to up-regulate its trans-activation (and expression), and knockdown of HMGB2 in immature hematopoietic progenitor cells leads to decreased Gfi-1B expression (Laurent B et al., Blood. Jan. 21, 2010; 115(3):687-95. Epub Nov. 24, 2009). Thus, decreasing the activity or expression of HMGB2 in a HSC may be used to decrease the expression/activity of Gfi1b. Inhibitors of HMGB2 are known in the art. For example, siRNA directed against human HMGB2 are commercially available from Sigma-Aldrich (MISSION® siRNA, Cat. #SASI_Hs01_{—}00017264 to SASI_Hs01_{—}00017275) and Origene (Cat. #SR302141), and shRNA directed against human HMGB2 are also commercially available from Sigma-Aldrich (MISSION® shRNA Plasmid DNA, Cat. #SHCLND-NM_{—}002129) and Origene (Cat. #TG316577).

In embodiment, the above-mentioned inhibitor of Gfi1b (e.g., nucleic acid, polypeptide, peptide, antibodies, drugs) further comprises a moiety for increasing their entry into a cell and/or into the nucleus of a cell. Molecules or moieties capable of increasing the entry of macromolecules into a cell are well known in the art and include, for example peptides known as protein transduction domains (sometimes termed cell-penetrating peptides (CPP) or Membrane Translocating Sequences (MTS)), such as those found in the HIV-1 Transactivator of transcription (TAT) and the HSV-1 VP22 proteins, the homeodomain of Homeoproteins (e.g., Drosophila's Antennapedia homeodomain (AntpHD), Hox proteins), as well as other synthetic peptides (see, e.g., Beerens A M et al., Curr Gene Ther. October 2003; 3(5): 486-94). Also, the conjugatuon of macromolecules to certain lipids or glycolipids increases the hydrophobic character of the macromolecules and their lipid-solubility, thus faciliting their translocation across the cell membrane. Nuclear localization signals or sequences (NLS), which target a protein to the cell nucleus, are well known in the art.

In another aspect, the present invention provides a composition comprising the above-mentioned inhibitor of Gfi1b and a pharmaceutically acceptable carrier, diluent and/or excipient, for (i) increasing the number of hematopoietic stem cells (HSCs) in a biological system; (ii) increasing the number of HSCs in the bone marrow and/or peripheral blood of a subject; and/or (iii) increasing the repopulation of HSCs in an HSC transplant recipient.

Such compositions may be prepared in a manner well known in the pharmaceutical art. Supplementary active compounds can also be incorporated into the compositions. As used herein “pharmaceutically acceptable carrier” or “excipient” or “diluent” includes any and all solvents, buffers, dispersion media, coatings, antibacterial and antifungal agents, isotonic and absorption delaying agents, and the like that are physiologically compatible. The carrier can be suitable, for example, for intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol) administration (see Remington: The Science and Practice of Pharmacy by Alfonso R. Gennaro, 2003, 21th edition, Mack Publishing Company).

Formulations suitable for oral administration can consist of (a) liquid solutions, such as an effective amount of active agent(s)/composition(s) suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds/compositions of the invention include ethylenevinyl acetate copolymer particles, osmotic pumps, implantable infusion systems, and liposomes. Formulations for inhalation may contain excipients, (e.g., lactose) or may be aqueous solutions containing, for example, polyoxyethylene-9-lauryl ether, glycocholate and deoxycholate, or may be oily solutions for administration in the form of nasal drops, or as a gel.

For preparing pharmaceutical compositions from the compound(s)/composition(s) of the present invention, pharmaceutically acceptable carriers are either solid or liquid. Solid form preparations include powders, tablets, pills, capsules, cachets, suppositories, and dispersible granules. A solid carrier can be one or more substance, which may also act as diluents, flavoring agents, binders, preservatives, tablet disintegrating agents, or an encapsulating material.

In powders, the carrier is a finely divided solid, which is in a mixture with the finely divided active component. In tablets, the active component (an inhibitor of Gfi1b) is mixed with the carrier having the necessary binding properties in suitable proportions and compacted in the shape and size desired. The powders and tablets may typically contain from 5% or 10% to 70% of the active compound/composition. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use are prepared by dissolving the Gfi1b inhibitor in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

In embodiments, the pharmaceutical compositions are formulated to target delivery of the active agent (e.g., an inhibitor of Gfi1b) to a particular cell, tissue and/or organ, such as the bone marrow, which is enriched in HSCs, or the peripheral blood. For example, it is known that formulation of an agent in liposomes results in a more targeted delivery to the bone marrow while reducing side effects (Hassan et al., Bone Marrow Transplant. 1998; 22(9):913-8). Myeloid-specific antigens can also be used to target the bone marrow (Orchard and Cooper, Q. J. Nucl. Med. Mol. Imaging. 2004; 48(4):267-78). In embodiments, the pharmaceutical compositions are formulated to increase the entry of the agent into a cell and/or into the nucleus of a cell.

An “effective amount” is an amount sufficient to effect a significant biological effect, such as (i) increasing the number of hematopoietic stem cells (HSCs) in a biological system; (ii) increasing the number of HSCs in the bone marrow and/or peripheral blood of a subject; and/or (iii) increasing the repopulation of HSCs in an HSC transplant recipient. In an embodiment, the above-mentioned agent or composition is used in an effective amount so as to (i) increase the number of hematopoietic stem cells (HSCs) in a biological system; (ii) increase the number of HSCs in the bone marrow and/or peripheral blood of a subject; and/or (iii) increase the repopulation of HSCs in an HSC transplant recipient, by at least 10%, 20%, 30%, 40%, 50%, 60%, 70%, 80%, 90%, 100% (i.e. 2-fold), 3-fold, 4-fold, 5-fold, 10-fold, 20-fold, 30-fold, 50-fold or 100-fold. An effective amount can be administered in one or more administrations, applications or dosages. The compositions can be administered one from one or more times per day to one or more times per week; including once every other day. The skilled artisan will appreciate that certain factors may influence the dosage and timing required to effectively treat a subject, including but not limited to previous treatments, the general health and/or age of the subject, the target site of action, the patient's weight, special diets being followed by the patient, concurrent medications being used, the administration route, other diseases present and other factors. Moreover, treatment of a subject with a therapeutically effective amount of the compositions described herein can include a single treatment or a series of treatments. The dosage will be adapted by the clinician in accordance with conventional factors such as the extent of the disease and different parameters from the patient. Typically, 0.001 to 1000 mg/kg of body weight/day will be administered to the subject. In an embodiment, a daily dose range of about 0.01 mg/kg to about 500 mg/kg, in a further embodiment of about 0.1 mg/kg to about 200 mg/kg, in a further embodiment of about 1 mg/kg to about 100 mg/kg, in a further embodiment of about 10 mg/kg to about 50 mg/kg, may be used. The dose administered to a patient, in the context of the present invention should be sufficient to effect/induce a beneficial biological effect in the patient over time. The size of the dose also will be determined by the existence, nature, and extent of any adverse side-effects that accompany the administration. Effective doses may be extrapolated from dose response curves derived from in vitro or animal model test systems. For example, in order to obtain an effective mg/kg dose for humans based on data generated from rat studies, the effective mg/kg dosage in rat may be divided by six.

In embodiments, the methods include administering a combination of active agents, for example an inhibitor of Gfi1b in combination with an agent currently used in HSC-based therapies (e.g., in bone marrow and/or HSC transplantation). In an embodiment, the inhibitor of Gfi1b is used in combination with one or more agents used to increase HSC expansion and/or mobilization, such as granulocyte-colony stimulating factor (G-CSF), interleukin-17 (IL-17), cyclophosphamide (Cy), Docetaxel (DXT), or with an anti-rejection agent, such as immunosuppressive drugs. The above-mentioned inhibitor of Gfi1b may be formulated in a single composition with a second active agent, or in several individual compositions which may be co-administered in the course of the treatment. Co-administration in the context of the present invention refers to the administration of more than one active agent in the course of a coordinated treatment to achieve a biological effect and/or an improved clinical outcome. Such co-administration may also be coextensive, that is, occurring during overlapping periods of time. For example, a first agent may be administered to a patient before, concomitantly, before and after, or after a second active agent is administered. The agents may in an embodiment be combined/formulated in a single composition and thus administered at the same time.

The invention further provides a kit or package comprising the above-mentioned inhibitor of Gfi1b or the above-mentioned composition, together with instructions for (i) increasing the number of hematopoietic stem cells (HSCs) in a biological system; (ii) increasing the number of HSCs in the bone marrow and/or peripheral blood of a subject; and/or (iii) increasing the repopulation of HSCs in an HSC transplant recipient. The kit may further comprise, for example, containers, buffers, a device (e.g., syringe) for administering the inhibitor of Gfi1b or a composition comprising same to a subject.

The methods, uses, compositions and kits defined above may be useful for reconstituting the HSCs population in a patient in need of HSC renewal, for example for the treatment of patients affected with disorders, diseases, and/or conditions that would benefit from an increase in the number of HSCs, for example to reconstitute damaged or depleted hematopoietic system. Examples of disorders, diseases, and/or conditions contemplated for treatment by the present methods, uses, compositions and kits include diseases of the blood and bone marrow, such as cancers (e.g., leukemia, lymphoma, multiple myeloma), anemia (aplastic anemia, sickle-cell anemia), immunological disorders, thalassemia major, myelodysplastic syndrome, Blackfan-Diamond syndrome, globoid cell leukodystrophy, severe combined immunodeficiency (SCID), X-linked lymphoproliferative syndrome, and Wiskott-Aldrich syndrome. Patients that may benefit from treatments that utilize the present methods, uses, compositions and kits include candidates for bone marrow transplantation (“BMT”) and hematopoietic stem cell transplantation (“HSCT”), which patients are subjected to radiotherapy and/or chemotherapy regimen to eradicate or severely comprise the recipient's hematopoietic system before transplantation. Other diseases that may be treated through bone marrow transplants include: Hunter's syndrome, Hurler's syndrome, Lesch Nyhan syndrome, and osteopetrosis.

A HSC population obtained from a donor can be induced to proliferate ex vivo or in vitro, or an endogenous HSC population within a patient can be induced to proliferate in vivo or in situ by exposing the HSC population of interest to an agent that inhibit Gfi1b expression and/or activity.

In embodiments, the source of HSCs may be bone marrow, peripheral blood, cord blood (umbilical cord blood), amniotic fluid, fetal liver, or placental/fetal blood.

A given HSC population obtained from a donor or within a recipient host (i.e., a patient) can be induced to expand and/or to egress from the bone marrow by providing compounds/compositions that can inhibit Gfi1b expression and/or activity. For example, the compounds/compositions may be administered to a potential HSC transplant donor (an autologous or heterologous donor) to increase the number of HSCs in the peripheral blood prior to collecting the HSCs using standard methods (e.g., leukapheresis). The compounds/compositions may be administered to an HSC recipient to increase the number of HSCs in the peripheral blood following HSC transplantation. The compounds/compositions may also be used to increase the number of HSCs in a sample (e.g., in vitro or ex vivo) collected from a potential HSC or bone marrow donor. Thus, in embodiments, the methods and used described above further include obtaining a bone marrow and/or peripheral blood sample from a subject, using standard methods (e.g., bone marrow harvest, leukapheresis). The bone marrow and/or peripheral blood sample is maintained in vitro and contacted with an effective amount of an inhibitor of as described herein. The bone marrow and/or peripheral blood sample thus treated can be reintroduced into the subject (autologous transplantation), or transplanted/infused into a second subject, the transplant recipient (allogeneic transplantation), which is preferably HLA-matched with the donor.

Sources of human HSCs include peripheral blood. The HCSs could be mobilized to migrate from marrow to peripheral blood in greater numbers by treating the human donor with a cytokine, such as granulocyte-colony stimulating factor (G-CSF). In the following days, HSCs are collected, for example, based on size and density by counterflow centrifugal elutriation or any other methods known in the art see as equilibrium density centrifugation, velocity sedimentation at unit gravity, immune resetting and immune adherence, T lymphocyte depletion, and/or fluorescence-activated cell sorting (FACS) (see, e.g., Blood and marrow stem cell transplantation: principles, practice, and nursing insights. Marie Bakitas Whedon; Debra Wujcik, Sudbury, Mass.: Jones and Bartlett Publishers©, 1997, Jones and Bartlett series in oncology).

Expansion of HSCs in accordance with methods of the present invention can be performed by treating a HSC population with an effective amount of a Gfi1b inhibitor. When it is used to expand HSCs ex vivo or in vivo in a subject in need of such expansion (ex. subject needing a bone marrow/HSC transplantation, etc.), the expansion treatment with an inhibitor of Gfi1b may also further comprise at least one other active agent capable of directly or indirectly expanding HSCs and/or hematopoietic progenitor cells. Expansion of HSCs can be performed in a bioreactor such as the AastromReplicell™ system from Aastrom Biosciences (USA) or the Cytomatrix™ Bioreactor from Cytomatrix. It can also be performed using low molecular chelate for copper binding such as the StemEx™ from Gamida (Israel) or using culture systems such as MainGen (Germany) or culture medium such as ViaCell (USA). Examples of media used to culture hematopoietic stem cells include a minimum essential medium (MEM) containing about 5 to 20% bovine fetal serum, Dulbecco's modified Eagle medium (DMEM), RPMI 1640 medium, 199 medium and the like. As required, cytokines such as stem cell factor (SCF), interleukin-3 (IL-3), interleukin-6 (IL-6), interleukin-7 (IL-7), interleukin-11 (IL-11), fms-like tyrosine kinase-3 (Flt-3) ligand (FLT), erythropoietin (EPO), and thrombopoietin (TPO), hormones such as insulin, transportation proteins such as transferrin, and the like may further be contained in the medium.

Before transplantation, the number of stem cells may be tested by taking a sample from the stem cells (called pilot sample) and plating these stem cells on a methylcellulose agar complemented with the appropriate cytokines. After 10-20 days, the number of colonies is determined and this allows evaluating how many stem cells were present in the pilot sample. Knowing this number, it is possible to estimate the number of functional stem cells in the original sample.

The present invention also provides methods (in vitro or in vivo methods) for screening of test compounds, to identify compounds that may be useful for (i) increasing the number of hematopoietic stem cells (HSCs) in a biological system; (ii) increasing the number of HSCs in the bone marrow and/or peripheral blood of a subject; and/or (iii) increasing the repopulation of HSCs in an HSC transplant recipient. In general, the methods will include evaluating the effect of a test compound on the expression and/or activity of Gfi1b, or of a reporter protein, in a sample.

Accordingly, in another aspect, the present provides a method (in vitro or in vivo) for determining whether a test compound may be useful for (i) increasing the number of hematopoietic stem cells (HSCs) in a biological system; (ii) increasing the number of HSCs in the bone marrow and/or peripheral blood of a subject; and/or (iii) increasing the repopulation of HSCs in an HSC transplant recipient, said method comprising:
(a) contacting said test compound with a Gfi1b polypeptide or a fragment thereof having Gfi1b activity;
(b) determining whether said test compound binds to said Gfi1b polypeptide or fragment thereof;
   wherein the binding of said test compound to said Gfi1b polypeptide or fragment thereof is indicative that said test compound may be useful for (i) increasing the number of hematopoietic stem cells (HSCs) in a biological system; (ii) increasing the number of HSCs in the bone marrow and/or peripheral blood of a subject; and/or (iii) increasing the repopulation of HSCs in an HSC transplant recipient. In an embodiment, the method further comprises determining whether said test compound inhibits Gfi1b expression and/or Gfi1b activity.

In another aspect, the present provides a method (in vitro or in vivo) for determining whether a test compound may be useful for (i) increasing the number of hematopoietic stem cells (HSCs) in a biological system; (ii) increasing the number of HSCs in the bone marrow and/or peripheral blood of a subject; and/or (iii) increasing the repopulation of HSCs in an HSC transplant recipient, said method comprising:
(a) contacting said test compound with a cell exhibiting Gfi1b expression and/or activity;
(b) determining whether said test compound inhibits said expression and/or Gfi1b activity;
   wherein the inhibition of said Gfi1b expression and/or activity in the presence of said test compound is indicative that said test compound may be useful for (i) increasing the number of hematopoietic stem cells (HSCs) in a biological system; (ii) increasing the number of HSCs in the bone marrow and/or peripheral blood of a subject; and/or (iii) increasing the repopulation of HSCs in an HSC transplant recipient.

In another aspect, the present provides a method (in vitro or in vivo) for determining whether a test compound may be useful for (i) increasing the number of hematopoietic stem cells (HSCs) in a biological system; (ii) increasing the number of HSCs in the bone marrow and/or peripheral blood of a subject; and/or (iii) increasing the repopulation of HSCs in an HSC transplant recipient, said method comprising:
(a) contacting said test compound with a cell comprising a first nucleic acid comprising a transcriptional regulatory element normally associated with a Gfi1b gene, operably linked to a second nucleic acid encoding a reporter protein;
(b) determining whether reporter gene expression or activity is inhibited in the presence of said test compound;
   wherein the inhibition of said reporter gene expression or activity in the presence of said test compound is indicative that said test compound may be useful for (i) increasing the number of hematopoietic stem cells (HSCs) in a biological system; (ii) increasing the number of HSCs in the bone marrow and/or peripheral blood of a subject; and/or (iii) increasing the repopulation of HSCs in an HSC transplant recipient.

In another aspect, the present provides a method (in vitro or in vivo) for determining whether a test compound may be useful for (i) increasing the number of hematopoietic stem cells (HSCs) in a biological system; (ii) increasing the number of HSCs in the bone marrow and/or peripheral blood of a subject; and/or (iii) increasing the repopulation of HSCs in an HSC transplant recipient, said method comprising:
(a) contacting said test compound with a nucleic acid comprising a Gfi1b binding sequence (e.g., a sequence comprising TAAATCAC(A/T)GCA) in the presence of Gfi1b;
(b) determining whether said test compound inhibits the binding of Gfi1b to said nucleic acid;
   wherein the inhibition of the binding of Gfi1b to said nucleic acid in the presence of said test compound is indicative that said test compound may be useful for (i) increasing the number of hematopoietic stem cells (HSCs) in a biological system; (ii) increasing the number of HSCs in the bone marrow and/or peripheral blood of a subject; and/or (iii) increasing the repopulation of HSCs in an HSC transplant recipient.

In another aspect, the present invention provides a method (in vitro or in vivo) for determining whether a test compound may be useful for (i) increasing the number of hematopoietic stem cells (HSCs) in a biological system; (ii) increasing the number of HSCs in the bone marrow and/or peripheral blood of a subject; and/or (iii) increasing the repopulation of HSCs in an HSC transplant recipient, said method comprising:
(a) contacting said test compound with a cell comprising a first nucleic acid comprising a transcriptional regulatory element comprising a Gfi1b binding sequence, operably linked to a second nucleic acid encoding a reporter protein;
(b) determining whether reporter gene expression or activity is increased in the presence of said test compound (i.e. determining whether the test compound is able to block the transcription repressor activity of Gfi1b, thus resulting in an increase in the expression of the reporter gene);
   wherein the increase of said reporter gene expression or activity in the presence of said test compound is indicative that said test compound may be useful for (i) increasing the number of hematopoietic stem cells (HSCs) in a biological system; (ii) increasing the number of HSCs in the bone marrow and/or peripheral blood of a subject; and/or (iii) increasing the repopulation of HSCs in an HSC transplant recipient.

The above-noted screening method or assay may be applied to a single test compound or to a plurality or “library” of such compounds (e.g., a combinatorial library). Any such compounds may be utilized as lead compounds and further modified to improve their therapeutic, prophylactic and/or pharmacological properties for (i) increasing the number of hematopoietic stem cells (HSCs) in a biological system; (ii) increasing the number of HSCs in the bone marrow and/or peripheral blood of a subject; and/or (iii) increasing the repopulation of HSCs in an HSC transplant recipient.

Test compounds (drug candidates) may be obtained from any number of sources including libraries of synthetic or natural compounds, including peptide/polypeptide librairies, small molecule libraries, RNAi libraries. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means.

Screening assay systems may comprise a variety of means to enable and optimize useful assay conditions. Such means may include but are not limited to: suitable buffer solutions, for example, for the control of pH and ionic strength and to provide any necessary components for optimal activity and stability (e.g., protease inhibitors), temperature control means for optimal activity and/or stability, of Gfi1b, and detection means to enable the detection of its activity. A variety of such detection means may be used, including but not limited to one or a combination of the following: radiolabelling, antibody-based detection, fluorescence, chemiluminescence, spectroscopic methods (e.g., generation of a product with altered spectroscopic properties), various reporter enzymes or proteins (e.g., horseradish peroxidase, green fluorescent protein), specific binding reagents (e.g., biotin/(strept)avidin), and others.

As noted above, the invention further relates to methods (in vitro or in vivo) for the identification and characterization of compounds capable of decreasing Gfi1b gene expression. Such a method may comprise assaying Gfi1b gene expression in the presence versus the absence of a test compound. Such gene expression may be measured by detection of the corresponding RNA or protein, or via the use of a suitable reporter construct comprising one or more transcriptional regulatory element(s), such as a promoter, normally associated with a Gfi1b gene, operably-linked to a reporter gene (i.e., any gene whose expression and/or activity may be detected, e.g., enzymatically or fluorescently), such as a luciferase gene (see, for example, Vassen et al., Nucleic Acids Research, 2005, Vol. 33, No. 3: 987-998) or other genes whose expression and/or activity may be detected (e.g., chloramphenicol acetyltransferase (CAT), beta-D galactosidase (LacZ), beta-glucuronidase (gus), luciferase, or fluorescent proteins (e.g., GFP, YFP, CFP, dsRed).

A first nucleic acid sequence is “operably-linked” with a second nucleic acid sequence when the first nucleic acid sequence is placed in a functional relationship with the second nucleic acid sequence. For instance, a promoter is operably-linked to a coding sequence if the promoter affects the transcription or expression of the coding sequences.

Generally, operably-linked DNA sequences are contiguous and, where necessary to join two protein coding regions, in reading frame. However, since, for example, enhancers generally function when separated from the promoters by several kilobases and intronic sequences may be of variable lengths, some polynucleotide elements may be operably-linked but not contiguous. “Transcriptional regulatory element” is a generic term that refers to DNA sequences, such as initiation and termination signals, enhancers, and promoters, splicing signals, polyadenylation signals which induce or control transcription of protein coding sequences with which they are operably-linked. The expression of such a reporter gene may be measured on the transcriptional or translational level, e.g., by the amount of RNA or protein produced. RNA may be detected by for example Northern analysis or by the reverse transcriptase-polymerase chain reaction (RT-PCR) method (see for example Sambrook et al. (1989) Molecular Cloning: A Laboratory Manual (2nd edition), Cold Spring Harbor Laboratory Press, Cold Spring Harbor, N.Y., USA).

Protein levels may be detected either directly using affinity reagents (e.g., an antibody or fragment thereof (for methods, see for example Harlow, E. and Lane, D (1988) Antibodies: A Laboratory Manual, Cold Spring Harbor Laboratory Press, Cold Spring Harbor, N.Y.); a ligand which binds the protein) or by other properties (e.g., fluorescence in the case of green fluorescent protein) or by measurement of the protein's activity, which may entail enzymatic activity to produce a detectable product (e.g., with altered spectroscopic properties) or a detectable phenotype (e.g., alterations in cell growth/function). Suitable reporter genes include but are not limited to chloramphenicol acetyltransferase (CAT), beta-D galactosidase (LacZ), beta-glucuronidase (gus), luciferase, or fluorescent proteins (e.g., GFP, YFP, CFP, dsRed).

Gfi1b protein expression levels could be determined using any standard methods known in the art. Non-limiting examples of such methods include Western blot, tissue microarray, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microscopy, fluorescence activated cell sorting (FACS), flow cytometry, and assays based on a property of the protein including but not limited to DNA binding, ligand binding, or interaction with other protein partners.

Methods to determine Gfi1b nucleic acid (mRNA) levels are known in the art, and include for example polymerase chain reaction (PCR), reverse transcriptase-PCR (RT-PCR), in situ PCR, SAGE, quantitative PCR (q-PCR), in situ hybridization, Southern blot, Northern blot, sequence analysis, microarray analysis, detection of a reporter gene, or other DNA/RNA hybridization platforms. For RNA expression, preferred methods include, but are not limited to: extraction of cellular mRNA and Northern blotting using labeled probes that hybridize to transcripts encoding all or part of one or more of the genes of this invention; amplification of Gfi1b mRNA expressed using gene-specific primers, polymerase chain reaction (PCR), quantitative PCR (q-PCR), and reverse transcriptase-polymerase chain reaction (RT-PCR), followed by quantitative detection of the product by any of a variety of means; extraction of total RNA from the cells, which is then labeled and used to probe cDNAs or oligonucleotides encoding all or part of Gfi1b, arrayed on any of a variety of surfaces.

In embodiments, competitive screening assays may be done by combining a Gfi1b polypeptide, or a fragment thereof and a probe (e.g., a nucleic acid probe comprising a Gfi1b-binding sequence, such as TAAATCAC(A/T)GCA, SEQ ID NO: 19) to form a probe:Gfi1b binding domain complex in a first sample followed by adding a test compound. The binding of the test compound is determined, and a change, or difference in binding of the probe in the presence of the test compound indicates that the test compound is capable of binding to the Gfi1b binding domain and potentially modulating Gfi1b activity.

The binding of the test compound may be determined through the use of competitive binding assays. In this embodiment, the probe is labeled with an affinity label such as biotin. Under certain circumstances, there may be competitive binding between the test compound and the probe, with the probe displacing the candidate agent. In one case, the test compound may be labeled. Either the test compound, or a compound of the present invention, or both, is added first to the Gfi1b binding domain for a time sufficient to allow binding to form a complex.

The assay may be carried out in vitro utilizing a source of Gfi1b which may comprise a naturally isolated or recombinantly produced Gfi1b (or a variant/fragment thereof having Gfi1b activity), in preparations ranging from crude to pure. Such assays may be performed in an array format. In certain embodiments, one or a plurality of the assay steps are automated.

In embodiments, the assays described herein may be performed in a cell or cell-free format.

A homolog, variant and/or fragment of Gfi1b which retains Gfi1b activity (e.g., transcription repression activity) may also be used in the methods of the invention. A fusion protein comprising Gfi1b or a variant/fragment thereof having Gfi1b activity, fused to a second polypeptide, such as a fluorescent tag (or any tag facilitating detection of the fusion protein), may also be used to assess the effect of a test compound on Gfi1b activity and/or expression.

In an aspect, the present invention provides a method (in vitro or in vivo) for determining whether a test compound may be useful for (i) increasing the number of hematopoietic stem cells (HSCs) in a biological system; (ii) increasing the number of HSCs in the bone marrow and/or peripheral blood of a subject; and/or (iii) increasing the repopulation of HSCs in an HSC transplant recipient, said method comprising:
(a) contacting said test compound with a cell comprising a first nucleic acid comprising a transcriptional regulatory element comprising a Gfi1b binding sequence, operably linked to a second nucleic acid encoding a reporter protein;
(b) determining whether reporter gene expression or activity is increased in the presence of said test compound (i.e. determining whether the test compound is able to block the transcription repressor activity of Gfi1b, thus resulting in an increase in the expression of the reporter gene);
   wherein the increase of said reporter gene expression or activity in the presence of said test compound is indicative that said test compound may be useful for (i) increasing the number of hematopoietic stem cells (HSCs) in a biological system; (ii) increasing the number of HSCs in the bone marrow and/or peripheral blood of a subject; and/or (iii) increasing the repopulation of HSCs in an HSC transplant recipient.

In embodiment, the method includes determining whether the test compound affects Gfi1b-mediated transcriptional repression. Thus, the sample can include a Gfi1b binding/recognition sequence operably linked to a reporter gene, such as a gene encoding a fluorescent protein (e.g., green, red, blue, cyan or yellow fluorescent protein) or any other detectable gene product (e.g., luciferase, beta-galactosidase, chloramphenicol acetyltransferase (CAT)). The effect of the test compound on Gfi1b-mediated transcriptional repression of the reporter gene can be measured by determining expression of the reporter gene, e.g., by detecting fluorescent emission in the case of a fluorescent protein, in the presence or absence of the test compound.

### MODE(S) FOR CARRYING OUT THE INVENTION

The present invention is illustrated in further details by the following non-limiting examples.

### EXAMPLE 1

### Materials and Methods

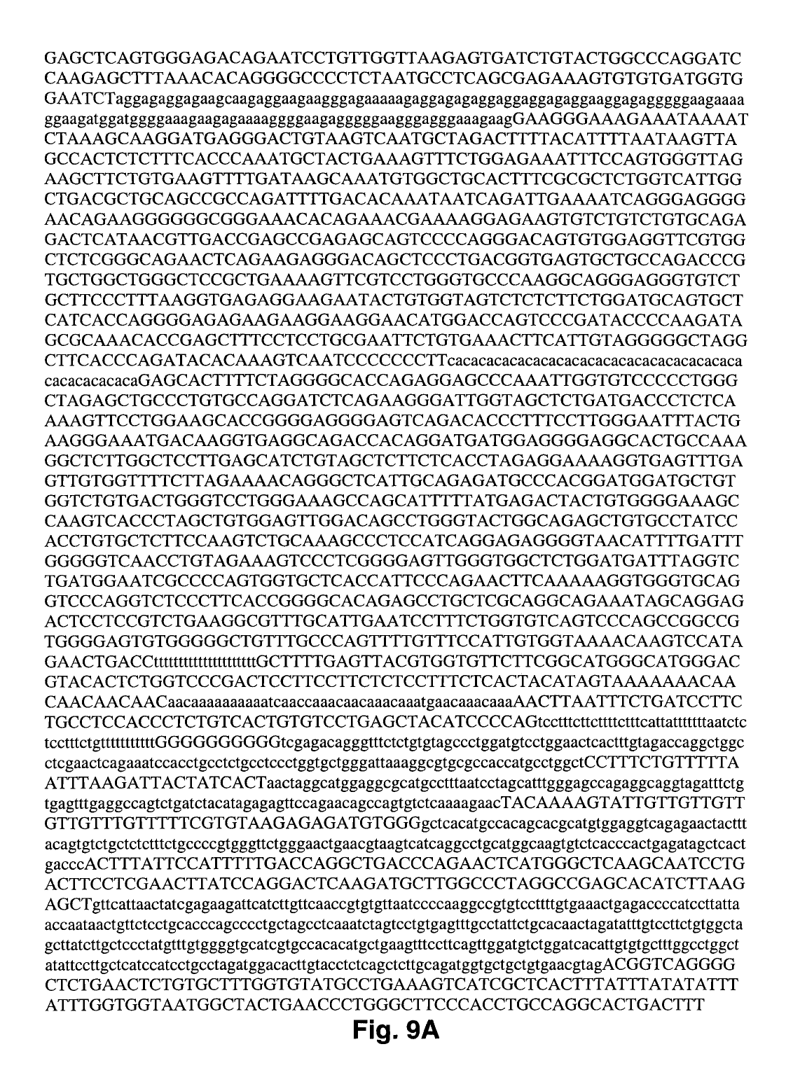

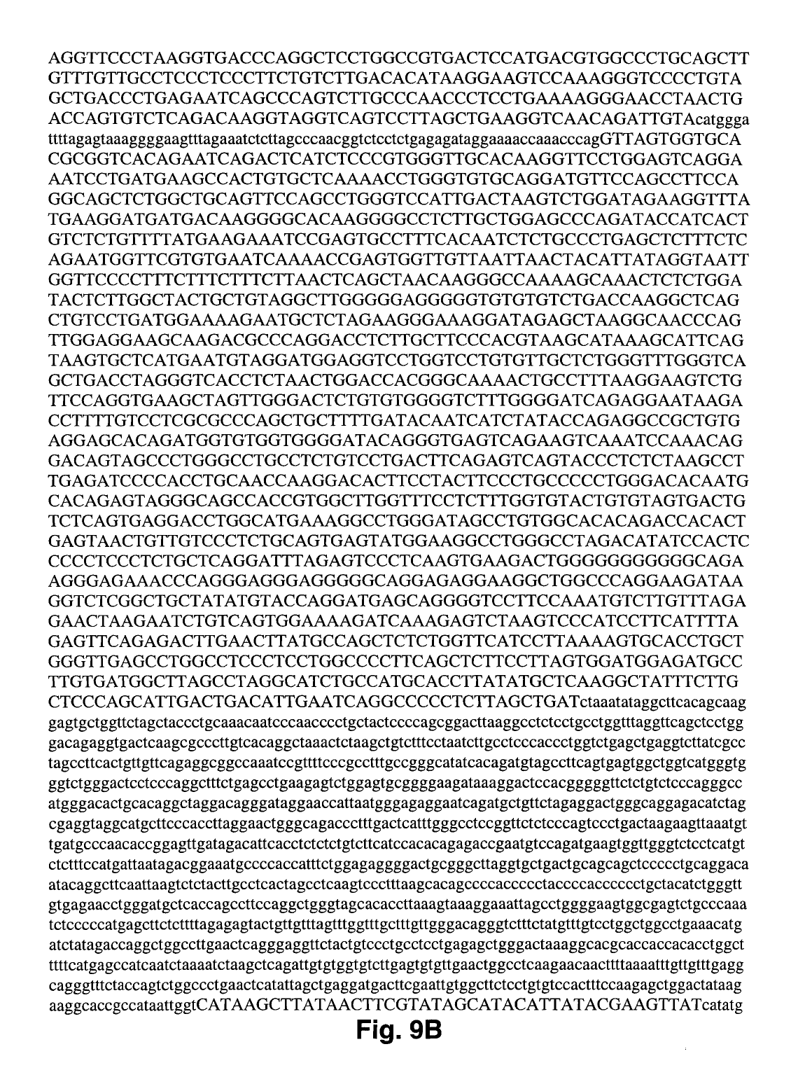

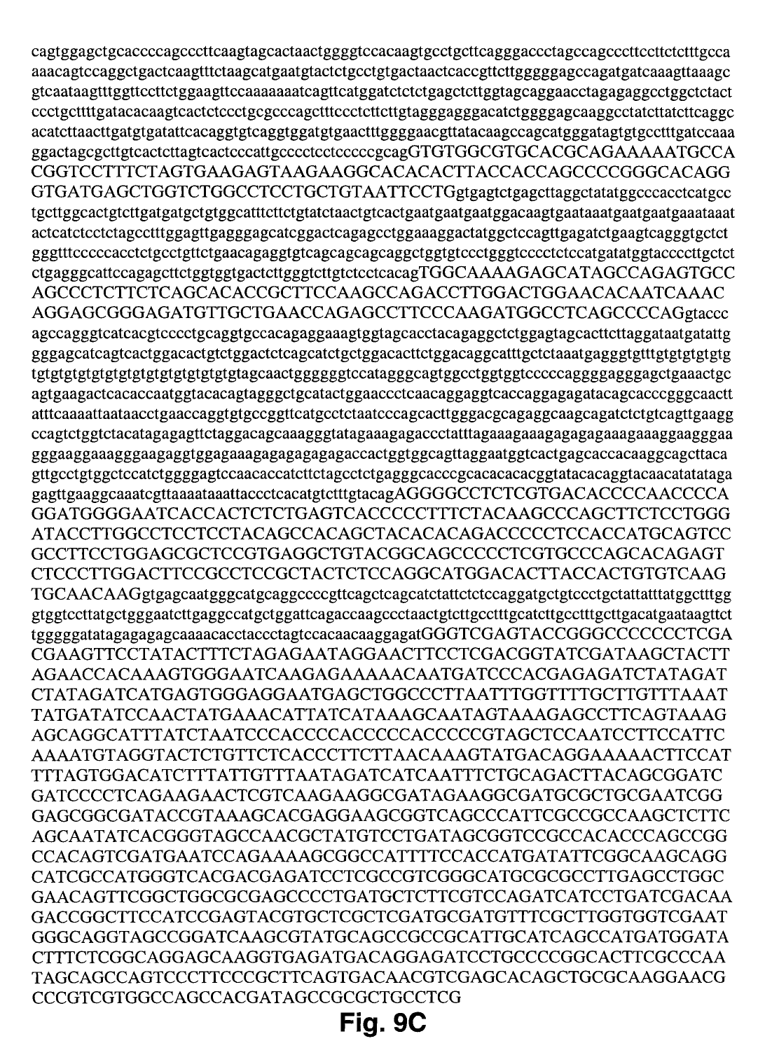

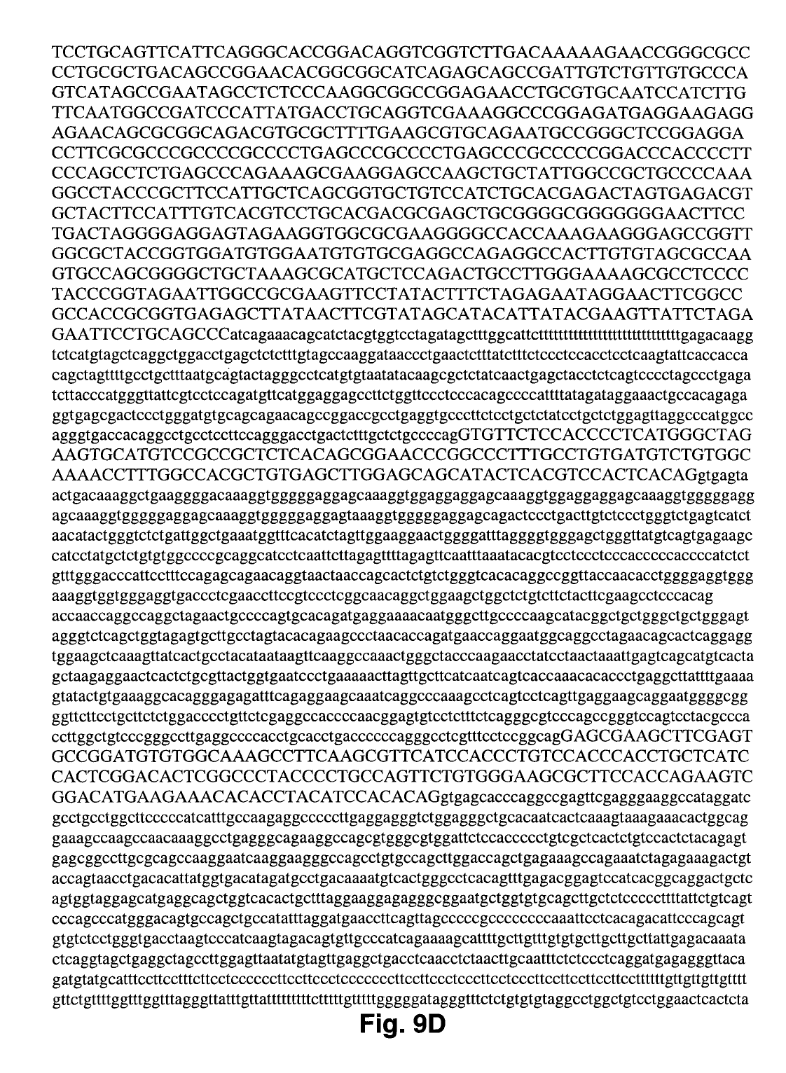

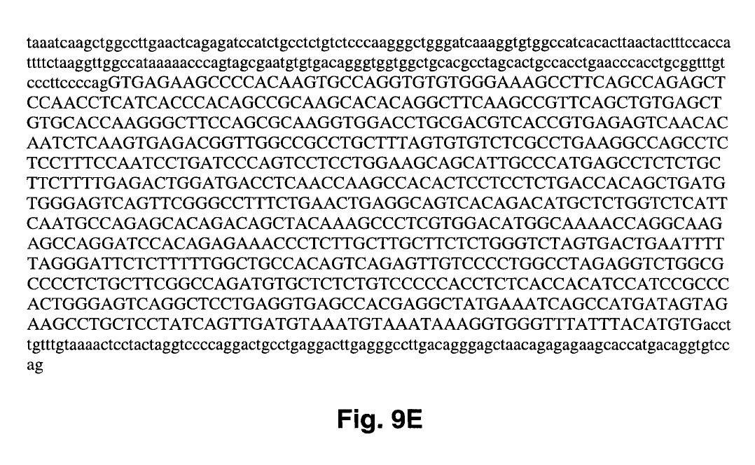

Mice. Gfi1b^{fl/fl} mice were generated by homologous recombination in R1 embryonic stem cells. The nucleotide sequence of the genomic-integrated part of the Gfi1b conditional knock-out construct is depicted in FIGS. 9A-9E (the sequences of the pBSII-SK+ plasmid backbone and the diphtheria toxin fragment A (DTA) selection marker are not shown, but the sequence of the PGK1-neo resistance gene is included). All mice were backcrossed with C57BI/6 mice and the C57BI/6 background was verified by specific satellite PCR. Gfi1^{fl/fl}, Gfi1^{GFP/WT} and Gfi1b^{GFP/WT} mice were described previously (Yucel R et al., J Biol Chem. 2004; 279:40906-40917; Vassen L et al. Blood. 2007; 109: 2356-2364; Zhu J et al. Proc Natl Acad Sci USA. 2006; 103:18214-18219). All mice were housed under (SPF) conditions.

Treatment. MxCre tg Gfi1^{fl/fl} or Gfi1b^{fl/fl} mice were injected with polyinosinic-polycytidylic acid (plpC) (Sigma-Aldrich) at a dose of 500 μg per injection every other day for a total of 5 injections. As control, wt or Gfi1b^{fl/fl} mice not carrying the MxCre tg were injected with plpC. With regard to N-Acetyl-Cystein (Sigma-Aldrich, Mississagua) treatment, mice were fed every day with 500 μl N-Acety-Cystein (50 mg/ml)

Flow cytometry analysis, sorting of HSC and progenitors. HSCs and progenitors were analyzed with a LSR™, or Cyan flow cytometers and HSC were sorted with a MoFlo™ from adult mouse bone marrow as described previously (Kiel et al., 2005, supra; Adolfsson J et al., Cell 2005; 121:295-306). The BrdU experiments and the determination of cell cycle phases by Hoechst staining was done according to described procedures (Wilson et al., 2005, supra). Reactive oxygen species (ROS) were analyzed by staining HSCs with 5-(and-6)-carboxy-2′,7′-dichlorodihydrofluorescein diacetate (carboxy-H2DCFDA) (Invitrogen, Burlington, Canada) for 30 min at 37° C. After staining, cells were analyzed by flow cytometry for level of ROS in HSCs.

Methylcellulose culture. 20,000 bone marrow cells were seed on methylcellulose (M3434, StemCell technologies, Vancouver, Canada) supplemented with EPO, IL-3, IL-6 and SCF. After 10 days, the number of colonies was determined. Subsequently, cells were resuspended and 10,000 cells of the suspension were replated on fresh methylcellulose medium.

Transplantation. The number of functional stem cells was determined in vivo using a limiting dilution assay, as described previously (Akala O O et al., Nature 2008; 453:228-232). Different amounts bone marrow cells from plpC-treated Gfi1b^{fl/fl} and MxCre tg Gfi1b^{fl/fl} mice (both CD45.2⁺) were transplanted together with 200,000 CD45.1⁺ bone marrow cells into lethally irradiated CD45.1⁺ mice. 18 weeks after transplantation, the peripheral blood of the recipient mice was analyzed for the contribution of CD45.2⁺ cells and a percentage of higher than 1% was considered a positive call. Using the L-Calc™ software from Invitrogen, the frequency of functional stem cells was determined.

PCR genotyping. Genotyping of Gfi1b^{fl/fl} mice was performed using the following primers:

| (SEQ ID NO: 10) | |
|---|---|
| LP5-3s: | GGTTTCTACCAGTCTGGCCCTGAACTC; |
| (SEQ ID NO: 11) | |
| LP3-3r: | CTCACCTCTCTGTGGCAGTTTCCTATC; |
| (SEQ ID NO: 12) | |
| LP5-4r: | TACATTCATGCTTAGAAACTTGAGTC. |

The product length of the wt allele is 255 bp, 295 bp for the floxed allele and 540 bp for the deleted allele.

Microarray Studies. Microarray data have been deposited in the GEO database (Accession No.=20655). Samples were hybridized with Affymetrix™ Mouse Gene 1.0 ST Arrays. Data was processed using the Affymetrix™ Expression Console software; algorithm-name: rma-gene-default. Only genes up- or down-regulated more than 2 times were taken into consideration.

Statistical Analysis. The unpaired Student t-test was chosen for analyzing the differences in the number of HSCs, CMPs, GMPs and platelets. ANOVA was used to compare plating efficiency between wt and Gfi1b-deficient bone marrow cells. All p-values were calculated two-sided, and values of p<0.05 were considered statistically significant. Statistical analysis was done with GraphPad™ Prism software (GraphPad software, La Jolla, Calif., USA).

### EXAMPLE 2

### Gfi1b is Highly Expressed in HSCs and Loss of Gfi1b Drastically Increases HSC Numbers

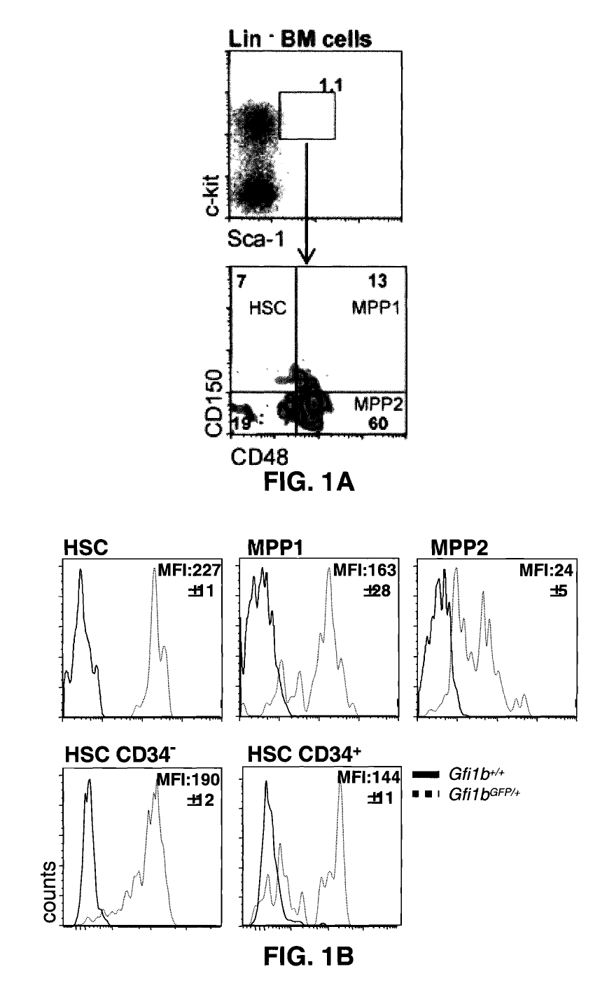

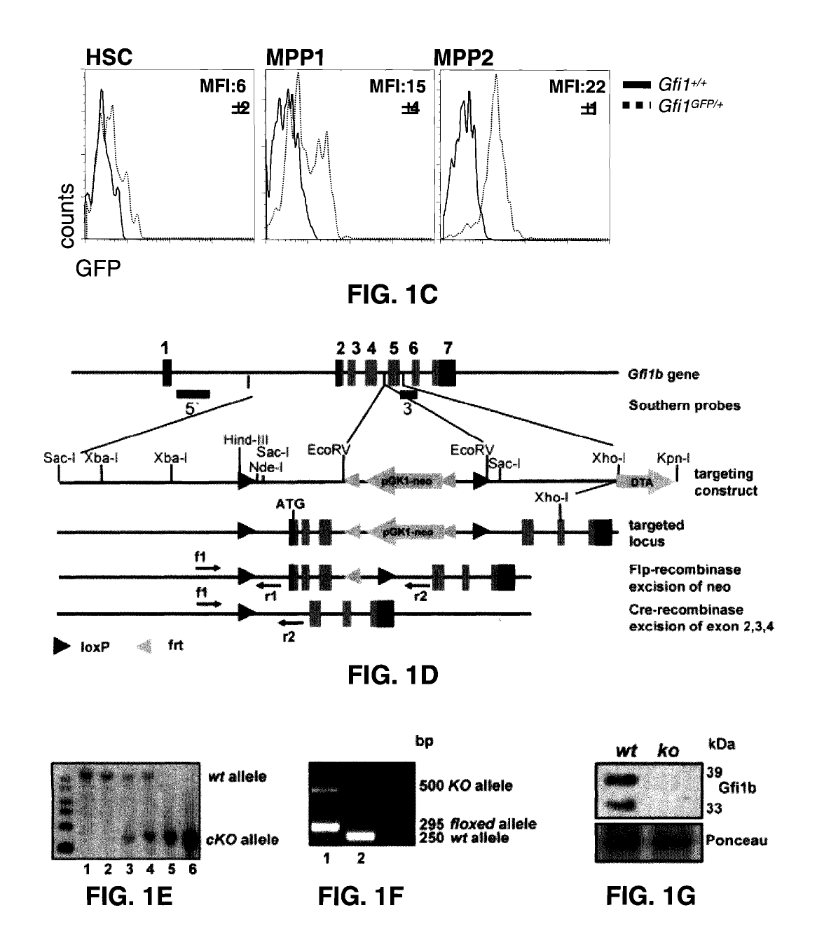

Using previously described Gfi1b:GFP knock-in mice (Gfi1b^{GFP/+}), in which the level of GFP follows Gfi1b promoter activity and Gfi1b mRNA levels (Vassen et al., 2007, supra), it was observed that Gfi1b is highly expressed in virtually all HSCs (defined as: Lin⁻, Sca-1⁺, c-kit⁺, (LSK), CD150⁺, CD48⁻) but is significantly down-regulated in the more differentiated MPP subsets (defined as MPP1: Lin⁻, Sca-1⁺, c-kit⁺, (LSK), CD150⁺, CD48⁺ and MPP2: Lin⁻, Sca-1⁺, c-kit⁺, (LSK), CD150⁻, CD48⁺) (FIGS. 1A and 1B). The dormant CD34⁻ HSC fraction (Wilson et al., 2005, supra) from Gfi1b^{GFP/WT} mice showed similar mean fluorescence intensities (MFI) than the activated CD34⁺ HSCs (FIG. 1B). In addition, using similar reporter mice for Gfi1 (Gfi1:GFP knock-in mice (Gfi1^{GFP/+}), in which the Gfi1 promoter activity and mRNA levels can be measured by monitoring green fluorescence (Yucel R et al., J Biol Chem. 2004; 279:40906-40917; Vassen et al., 2007, supra)), it was determined that expression levels of Gfi1 and Gfi1b were different in HSC and MPP subsets. In particular, the Gfi1b gene is highly expressed in HSCs and downregulated upon differentiation to the MPPs (FIGS. 1B, 1C), whereas Gfi1 shows lowest levels in HSCs and is upregulated in the MPP fractions, pointing to the possibility that both transcription factors are differentially regulated and have different roles in these cells.

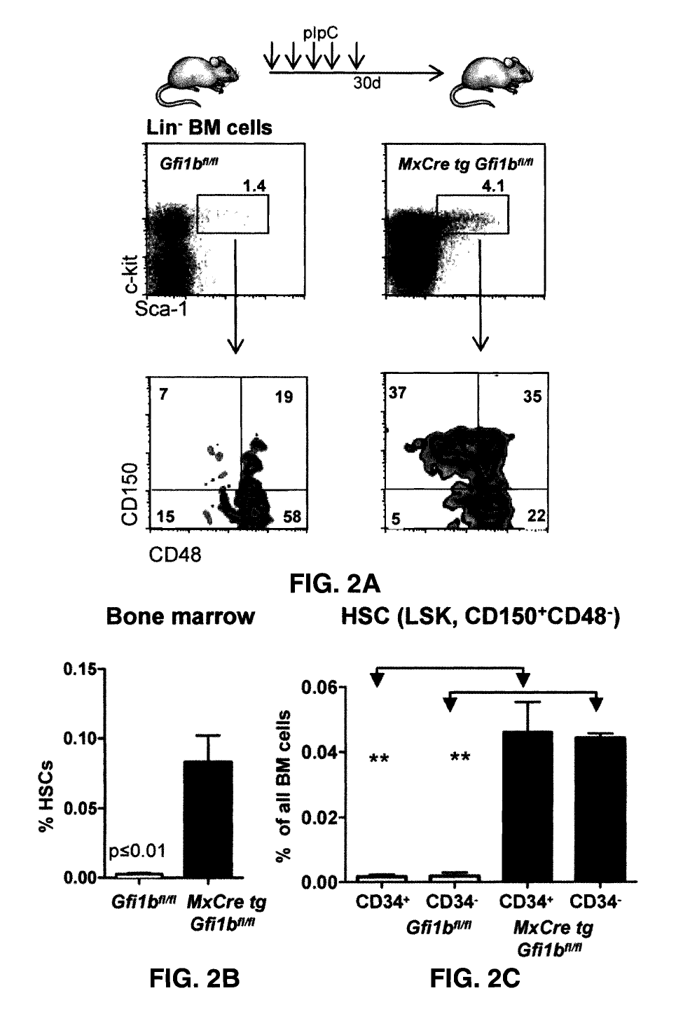

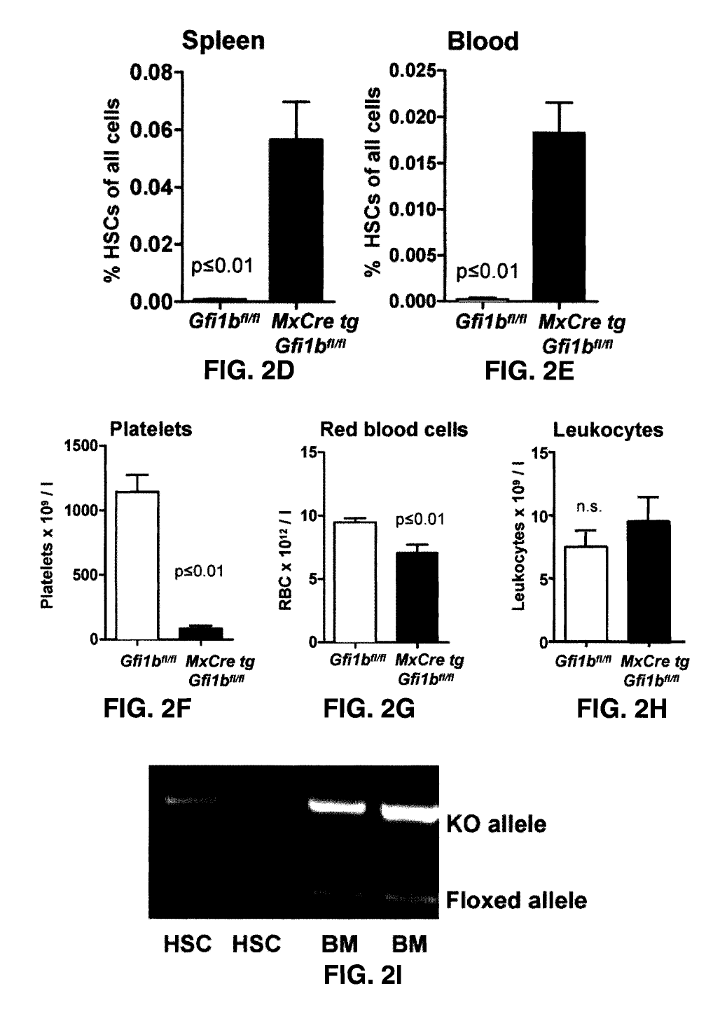

It was investigated whether Gfi1b plays a particular role, different from Gfi1, in HSCs. Since constitutively deficient Gfi1b mice die at mid-gestation (Saleque S et al., Genes Dev. 2002; 16:301-306) and thus cannot be used for analysing adult HSCs, a Gfi1b conditional mouse carrying floxed Gfi1b alleles and an MxCre transgene was generated (FIG. 1D). In these MxCre tg Gfi1b^{fl/fl} mice, Gfi1b exons 2-4 can be deleted after injection of plpC, leading to the abrogation of Gfi1b expression (FIGS. 1E to 1G). In order to exclude possible effects of plpC and interferon-alpha on HSCs, MxCre Gfi1b^{fl/fl} and Gfi1b^{fl/fl} mice were examined 20 days after the last plpC injection. It has been shown that this time period is sufficient to wean off effects of plpC or interferon-alpha on HSCs (Essers M A et al. Nature. 2009; 458:904-908). As shown in FIGS. 2A to 2E and Table 1, Gfi1b-deficient mice show increased frequencies of HSCs in bone marrow, spleen and in the peripheral blood (between 30- to 100-fold, respectively) relative to wild-type mice, a feature that is not observed in Gfi1-deficient mice (Zeng H et al. EMBO J. 2004; 23:4116-4125; Hock H et al. Nature. 2004; 431:1002-1007). The expansion affected both short-term (defined as CD34⁺ LSK, CD150⁺, CD48⁻) and long-term (CD34⁻ LSK, CD150⁺, CD48⁻) HSCs (FIG. 2C, Table 1).

TABLE 1
Change of hematological compartments and cell populations after
Gfi1b deletion

| | | Gfi1b^{fl/fl} | fold | |
|---|---|---|---|---|
| | Gfi1b^{fl/fl} | Mx-Cre tg | change | p-value |
| Number of BM | 36 ± 9, | 41 ± 13, | 1.13 | 0.21 |
| cells × 10⁶ | (n = 28) | (n = 27) | | |
| Number of | 94 ± 9, | 180 ± ,25 | 2 | 0.04 |
| splenocytes × 10⁶ | (n = 5) | (n = 9) | | |
| % Lin⁻ cells | 1.9 ± 0.3, | 3.1 ± 0.4, | 1.63 | 0.002 |
| in BM | (n = 14) | (n = 14) | | |
| Number of Lin⁻ | 0.7 ± 0.1, | 1.5 ± 0.2, | 2 | 0.01 |
| cells × 10⁶ | (n = 14) | (n = 14) | | |
| Number of HSCs | 1 000 ± 115, | 39000 ± 11000, | 39 | 0.0001 |
| in BM | (n = 14) | (n = 14) | | |
| Number of HSCs | 442 ± 150, | 51 000 ± 12800, | 115 | 0.01 |
| in Spleen | (n = 3) | (n = 5) | | |
| Number of HSCs | 15 ± 13, | 1435 ± 200, | 95 | 0.01 |
| per 1 ml blood | (n = 6) | (n = 6) | | |
| Number of CD34⁺ | 1100 ± 500, | 28000 ± 9000, | 25 | 0.04 |
| HSC | (n = 3) | (n = 3) | | |
| Number of CD34⁻ | 780 ± 280, | 25500 ± 500, | 32 | 0.001 |
| HSC | (n = 3) | (n = 3) | | |

The number of bone marrow (BM) cells, splenocytes and % of Lin⁻ cells was determined in wt and Gfi1b deficient mice.
The increase in number of splenocytes is mostly due to increase of number of erythroid progenitors.
HSCs are defined by immunophenotype as Lin⁻, Sca-1⁺, Kit⁺, CD150⁺, CD48⁻.
Depicted are Mean values, SEM and number of samples.
P-values are based on unpaired two-sided t-test.

The deletion of Gfi1b increased the number of Lin⁻ cells in the bone marrow but did not significantly alter the overall cellularity of the bone marrow (Table 1). In contrast there was an increase in the number of splenocytes in Gfi1b-deleted mice (Table 1), which was mainly the result of an expansion of erythroid progenitors in the spleen. Since the total number of bone marrow cells was not altered, the increased frequencies of HSCs correlated well with the increased absolute numbers of HSCs in bone marrow, spleen and blood indicating and expansion between 39- and over 100-fold, respectively (Table 1). It was also found that the number of platelets and erythrocytes in the peripheral blood was reduced compared to wt mice, albeit to different extents, whereas the total number of leukocytes was not changed (FIGS. 2F-2H). This is consistent with the established role of Gfi1b in the erythroid-megakaryocytic lineage (Anguita E et al., Haematologica January 2010; 95(1):36-46; Hernandez A, et al., Ann Hematol. August 2010; 89(8):759-765; Laurent B, et al. Blood. Jan. 21 2010; 115(3):687-695; Osawa M, et al. Blood. Oct. 15 2002; 100(8):2769-2777; Randrianarison-Huetz V et al. Blood. Apr. 8 2010; 115(14):2784-2795; Garcon L, et al. Blood. Feb. 15 2005; 105(4):1448-1455; Huang D Y et al. Nucleic Acids Res. 2004; 32(13):3935-3946; Saleque S, et al. Mol Cell. Aug. 17 2007; 27(4):562-572; Saleque S, et al. Genes Dev. Feb. 1 2002; 16(3):301-306). Finally, it was also verified whether the excision of the floxed Gfi1b regions was efficient in HSCs after plpC induction and observed that cells with non-excised Gfi1b alleles were below detection level (FIG. 2I).

### EXAMPLE 3

### HSCs from Gfi1b-Deficient Mice are Less Quiescent that wt HSCs and Contain More Reactive Oxygen Species (ROS)

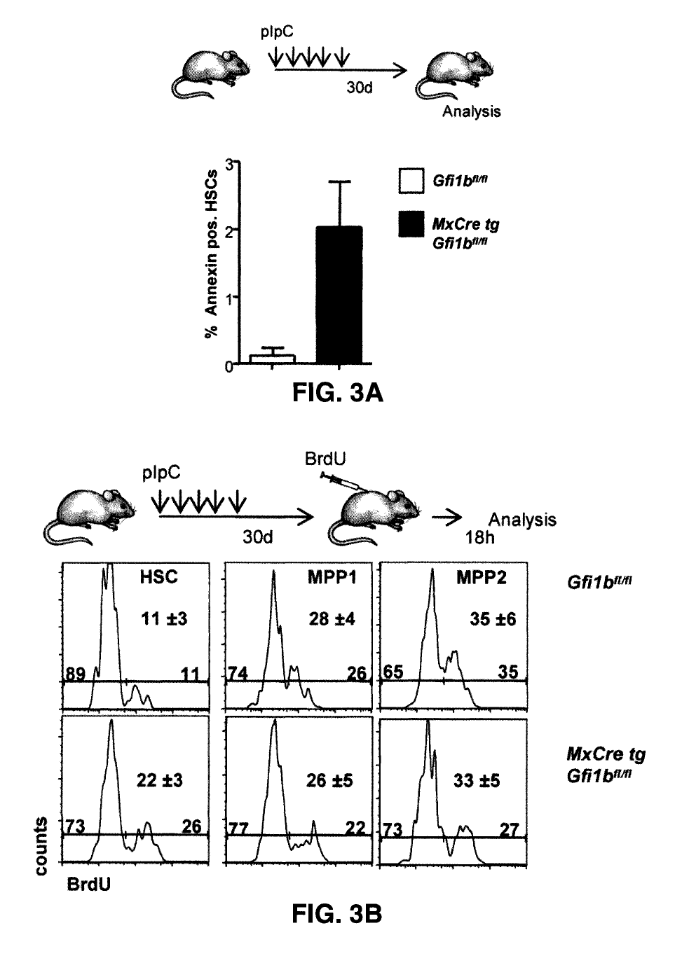

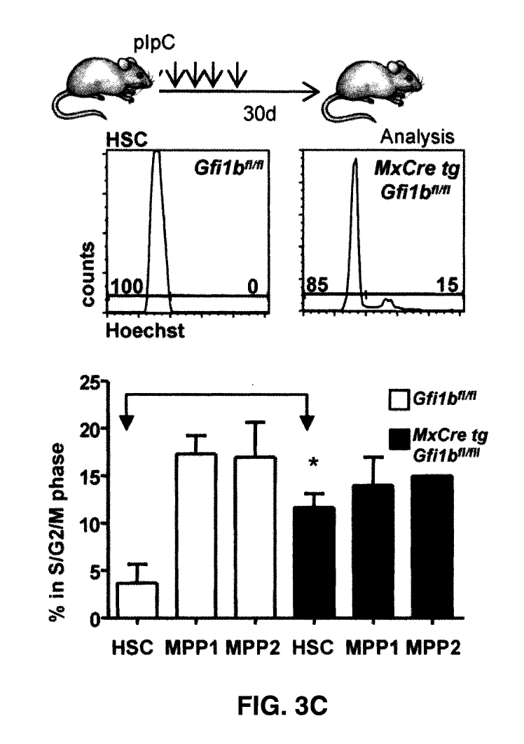

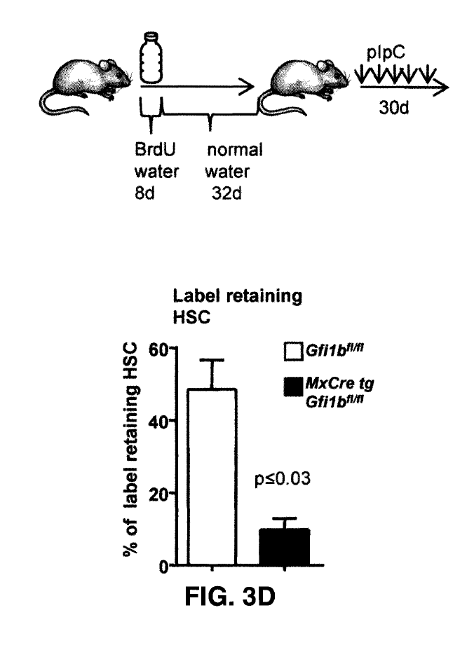

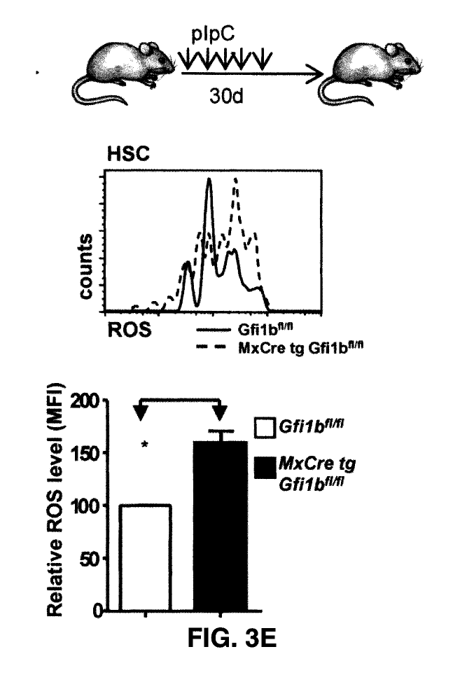

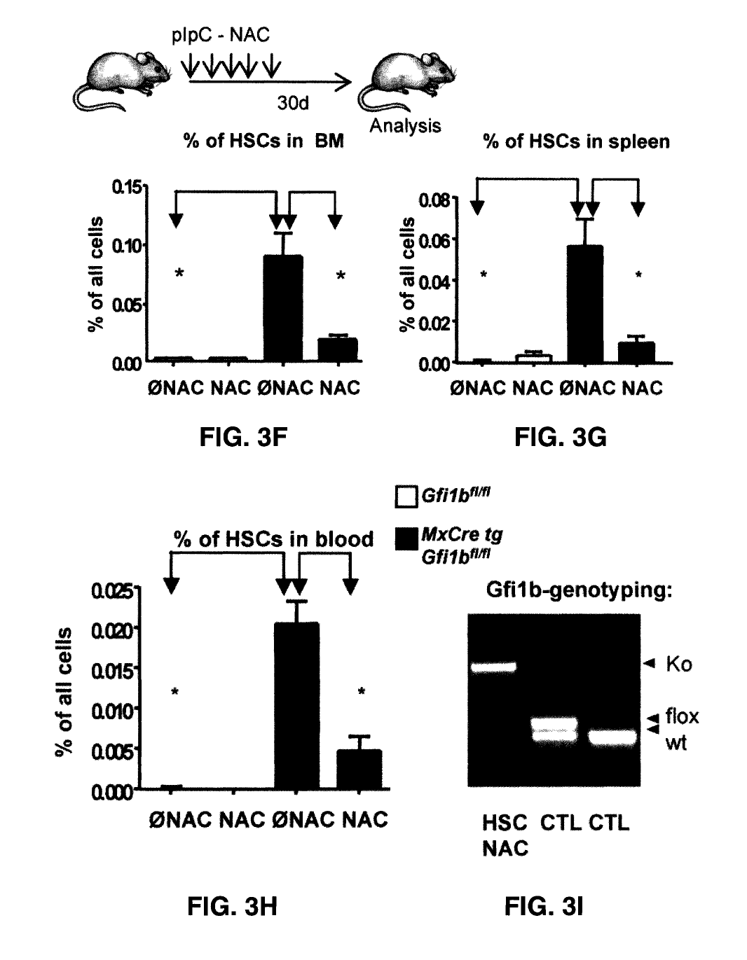

The increased numbers of HSCs in Gfi1b-deficient mice could be the result of a lower rate of spontaneous cell death or more proliferation. Gfi1b deficient (Gfi1b^{ko/ko}) HSCs underwent a slightly higher rate of spontaneous apoptosis than wt HSCs, but remained still under 2.5% (FIG. 3A). Using a BrdU pulse chase approach, it was found that the loss of Gfi1b correlated with increased frequencies of cycling HSCs, but had no or little effect on cells from the MPP subsets (FIG. 3B). Staining with Hoechst showed that Gfi1b^{ko/ko} mice had a higher percentage of HSCs in S and G2/M phases than wt mice (FIG. 3C), but that cell cycle progression of the MPPs was not altered. These two results indicate that Gfi1b restricts specifically the proliferation of HSCs and hence might control HSCs dormancy, but does not affect the rate of cell cycle progression in the different MPP fractions (FIG. 3c). In support of this, a label retention assay showed that only 10% of Gfi1b^{ko/ko} HSCs were quiescent, i.e., did not divide during the observation period (FIG. 3D). In contrast, 45% of the plpC-treated wt HSCs did not undergo a cell division at the end of the same time period (FIG. 3D). These findings indicates that a significant proportion of Gfi1b^{ko/ko} HSCs is no longer dormant and has entered the cell cycle. HSCs are kept in a dormant state at the endosteal niche, which provides a hypoxic environment and protects them against oxidative damage by reactive oxygen species (ROS), whereas high ROS are characteristic for activated HSCs and MPPs (Eliasson P and Jonsson J I. J Cell Physiol. 2010; 222:17-22; Arai F and Suda T. Ann NY Acad Sci. 2007; 1106:41-53. As shown in FIG. 3E, Gfi1b^{ko/ko} HSCs had a significantly increased level of ROS, when compared to the wt HSC population.

To verify whether loss of Gfi1b activates HSCs and that this activation leads to increased level of ROS which in turn could lead to an expansion of HSCs, mice were fed with N-Acetyl-Cystein (NAC), which counteracts the effects of ROS (Ito K, et al. Nat Med. April 2006; 12(4):446-451). It was found that administration of NAC significantly limited the expansion of Gfi1b^{ko/ko} HSCs in the bone marrow, spleen and peripheral blood both with regard to frequencies and absolute numbers (FIGS. 3F to 3H, Table 2) but did not affect the plpC-mediated excision of the floxed Gfi1b exons in HSCs (FIG. 3I). This indicated that elevated levels of ROS are at least partially responsible for the expansion of Gfi1b-deficient HSCs.

TABLE 2
Change of hematological compartments and cell populations after Gfi1b
deletion and N-Acetyl-Cystein injection

| | | Gfi1b^{fl/fl} | fold | |
|---|---|---|---|---|
| | Gfi1b^{fl/fl} | Mx-Cre tg | change | p-value |
| Number of BM | 45 ± 4, | 44 ± 2, | 1 | 0.8 |
| cells × 10⁶ NAC | (n = 7) | (n = 6) | | |
| treatment | | | | |
| Number of | 86 ± 22, | 104 ± 22, | 1.2 | 0.5 |
| splenocytes × 10⁶ | (n = 4) | (n = 5) | | |
| NAC treatment | | | | |
| % Lin⁻ cells | 1.5 ± 0.1, | 2.45 ± 0.5, | 1.6 | 0.15 |
| in BM NAC treatment | (n = 7) | (n = 6) | | |
| Number of Lin⁻ | 0.8 ± 0.1, | 1.1 ± 0.4, | 1.4 | 0.32 |
| cells × 10⁶ NAC | (n = 7) | (n = 6) | | |
| treatment | | | | |
| Number of HSCs | 1700 ± 700, | 5700 ± 1100, | 3 | 0.01 |
| in BM NAC treatment | (n = 7) | (n = 6) | | |
| Number of HSCs | 197 ± 77, | 6000 ± 2000, | 30 | 0.07 |
| in Spleen NAC | (n = 3) | (n = 4) | | |
| treatment | | | | |
| Number of HSCs | 11 ± 1, | 307 ± 100, | 28 | 0.03 |
| per 1 ml blood | (n = 3) | (n = 5) | | |
| NAC treatment | | | | |

The number of bone marrow (BM) cells, splenocytes and % of Lin⁻ cells was determined in wt and Gfi1b deficient mice.
Mice were fed daily with N-Acetyl-Cystein. HSCs are defined by immunophenotype as Lin⁻, Sca-1⁺, Kit⁺, CD150⁺, CD48⁻.
Depicted are Mean values, SEM and number of samples.
P-values are based on unpaired two-sided t-test.

### EXAMPLE 4

### Loss of Gfi1b Does Not Affect the Multipotency or Self-Renewal Capacity of HSCs

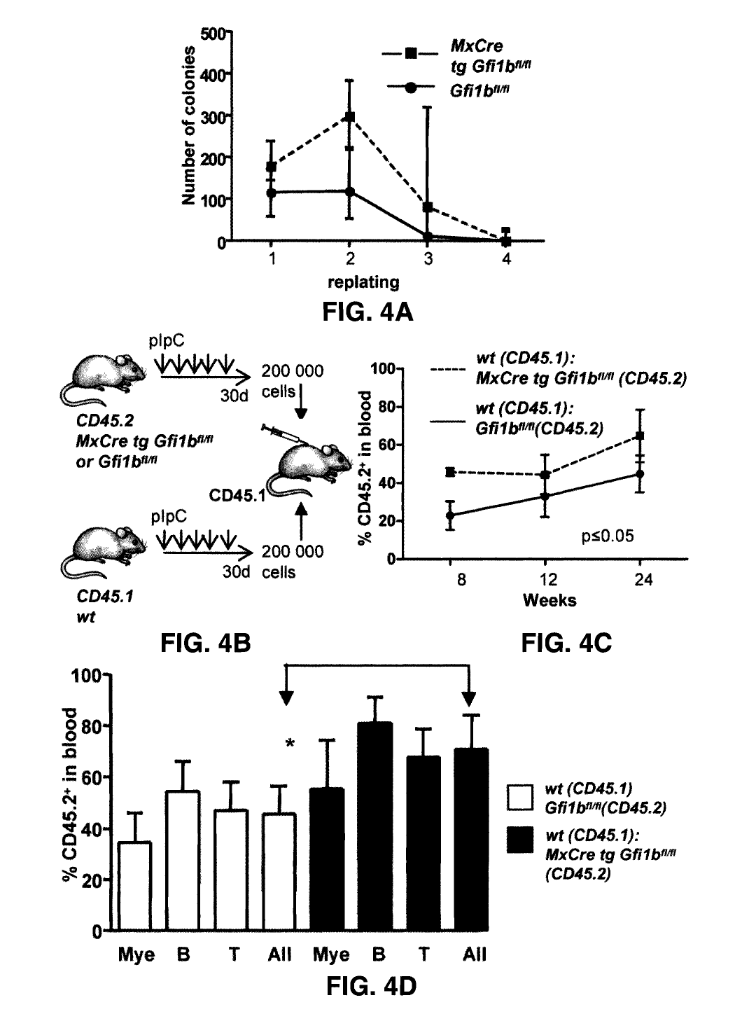

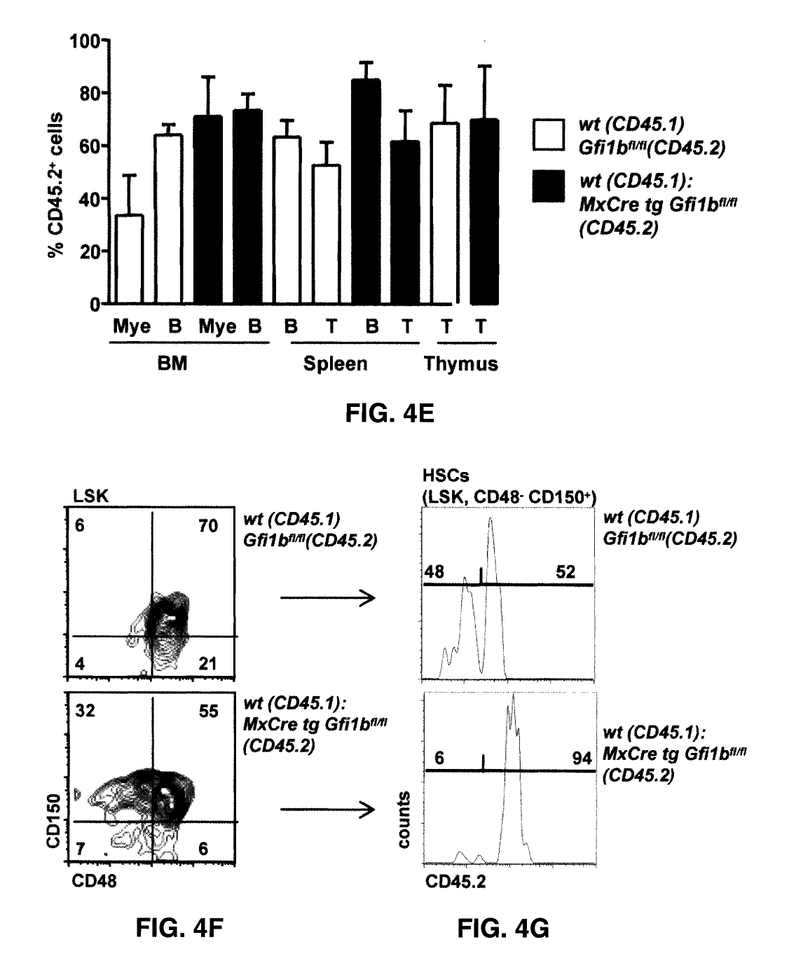

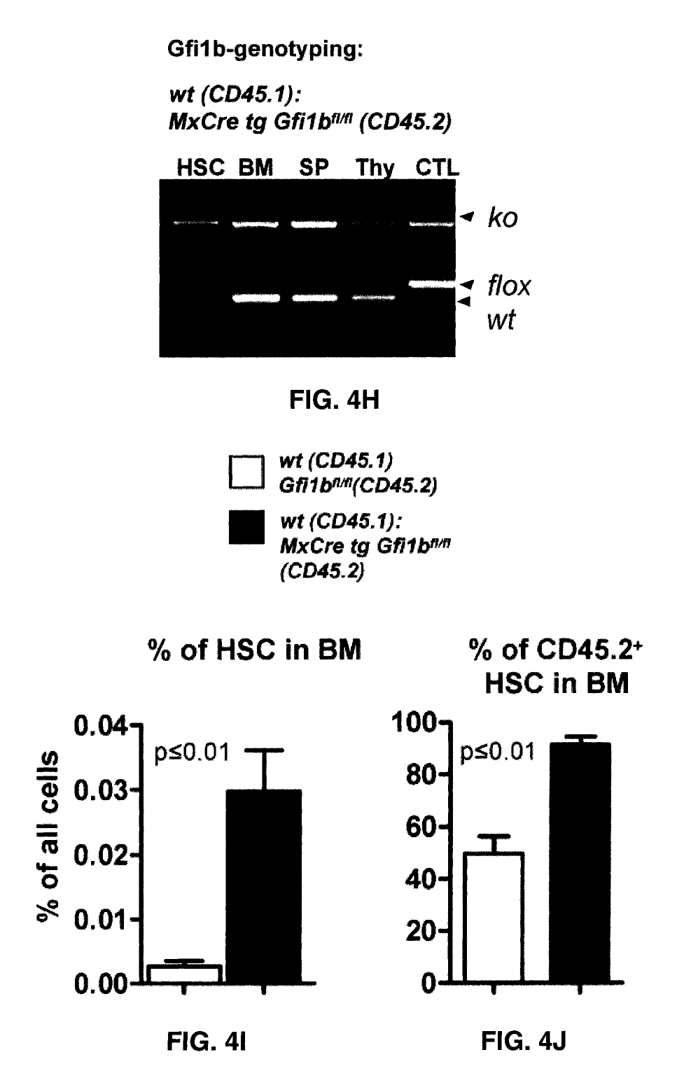

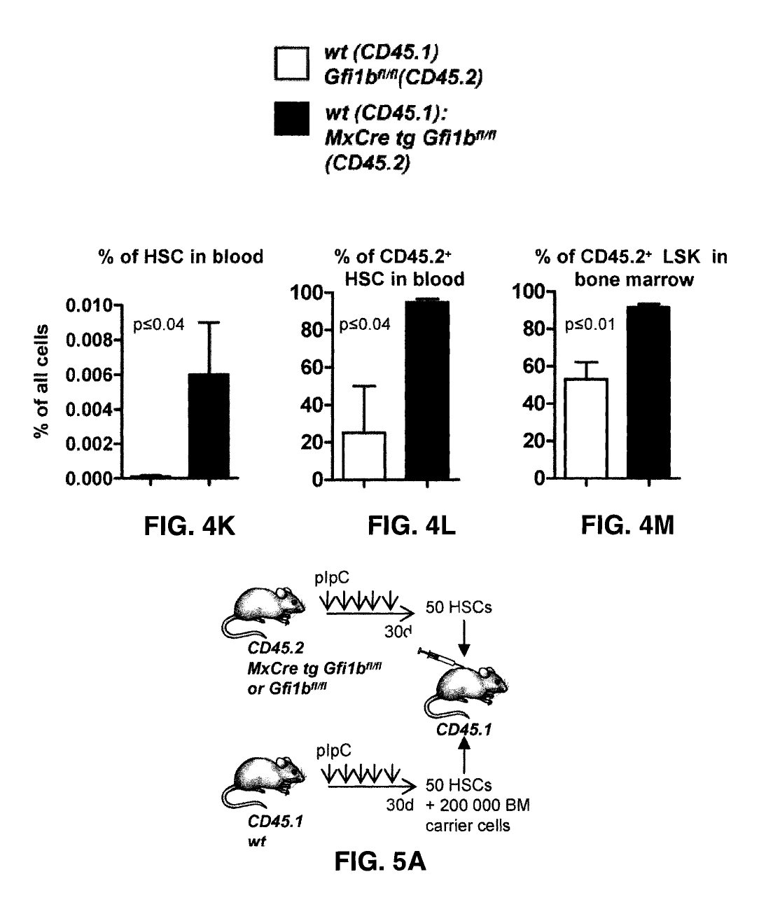

Next, it was investigated whether loss of Gfi1b might change the self-renewal capacity of HSCs. Gfi1b^{ko/ko} bone marrow cells generated the same type of colonies (including CFU-E, BFU-E, CFU-G, CFU-M, CFU-GM, CFU-GEMM) as wt cells, when seeded in methylcellulose and showed initially a higher replating efficiency and generated a higher number of colonies than wt bone marrow (FIG. 4A), which is in contrast to findings for Gfi1 (Zeng H et al. 2004, supra; Hock H et al. 2004, supra). However, after the 4^{th} cycle, Gfi1b^{ko/ko} cells exhausted their replating ability similar to wt cells (FIG. 4A). A limiting dilution assay was also performed to verify the number of functional HSCs in vivo, and a HSCs frequency of 1/7,000 cells was detected in Gfi1b^{ko/ko} mice, as compared to 1/46,000 cells in wt mice (Tables 3 and 4, p≦0.03). These findings suggested that Gfi1b deficiency enhances the number of functional HSCs by a factor of about 6 to 7 (Table 4).

TABLE 3
Determination of functional stem cells by limiting dilution assay

| Genotype | Dose (# of cells) | Positive recipients |
|---|---|---|
| Wt | 200 000 | 3/3 |
| Wt | 100 000 | 3/3 |
| Wt | 20 000 | 1/3 |
| Wt | 10 000 | 0/3 |
| Wt | 5 000 | 0/3 |
| Gfi1bko | 200 000 | 3/3 |
| Gfi1bko | 100 000 | 3/3 |
| Gfi1bko | 20 000 | 3/3 |
| Gfi1bko | 5 000 | 1/3 |

The number of functional stem cells was determined in-vivo by limiting dilution.
Indicated number of plpC treated Gfi1b^{fl/fl} and MxCre tg Gfi1b^{fl/fl} (Gfi1b^{ko/ko}) (both CD45.2⁺) bone marrow cells were transplanted with 200 000 CD45.1⁺ bone marrow cells into lethally irradiated CD45.1⁺ mice.
About 18 weeks after transplantation, peripheral blood was examined for presence of CD45.2⁺ cells.
A percentage higher than 1% was a positive call.

TABLE 4
Determination of functional stem cells by limiting dilution assay

| | One functional stem cell | |
|---|---|---|
| Genotype | within | Upper and lower limit |
| Wt | 1:46 000 | 1:20 000-1:200 000 |
| MxCre tg Gfi1b^{fl/fl} | 1:7 000* | 1:2 000-1:23 000 |

Based on the results in Table 2 number of functional stem cells was determined.
*denotes a statistically significant difference with p ≦ 0.05.

To further examine whether loss of Gfi1b alters self-renewal and multipotency of HSCs, 200 000 bone marrow cells from either wt or Gfi1b-deficient CD45.2 mice were transplanted in competition with wt CD45.1 bone marrow cells (FIG. 4B). Transplanted Gfi1b-deficient bone marrow cells were able to compete with wt CD45.1 cells with regard to blood, bone marrow, spleen and thymus repopulation and recipient mice transplanted with Gfi1b-deficient bone marrow cells even showed a significantly higher level of chimerism (measured as the percentage of CD45.2⁺ cells) in blood than recipients that received wt CD45.2 cells (FIGS. 4C and D). However, when frequencies of CD45.2⁺ myeloid or lymphoid cells were measured in bone marrow, spleen and thymus, there was no difference between mice that had received wt or Gfi1b-deficient bone marrow (FIG. 4E). In addition, a strong and highly significant expansion of transplanted CD45.2⁺ Gfi1b deficient HSCs in blood and bone marrow was observed (FIGS. 4F to 4M). Gfi1b-deficient (CD45.2⁺) HSCs represented almost 90% of all HSCs in the recipient animals (FIGS. 4F to 4J). A similar expansion of Gfi1b-deficient HSCs was also detectable in the peripheral blood of recipients that received Gfi1b-deficient bone marrow indicating that the phenotype of HSCs expansion observed in mice lacking Gfi1b is cell autonomous (FIGS. 4K and 4L).

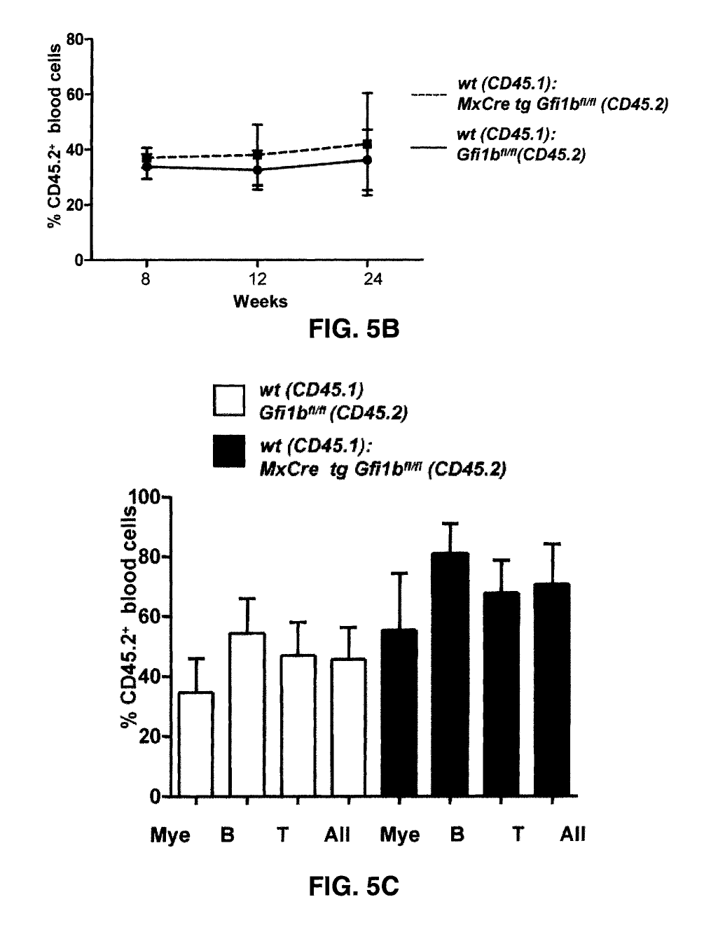

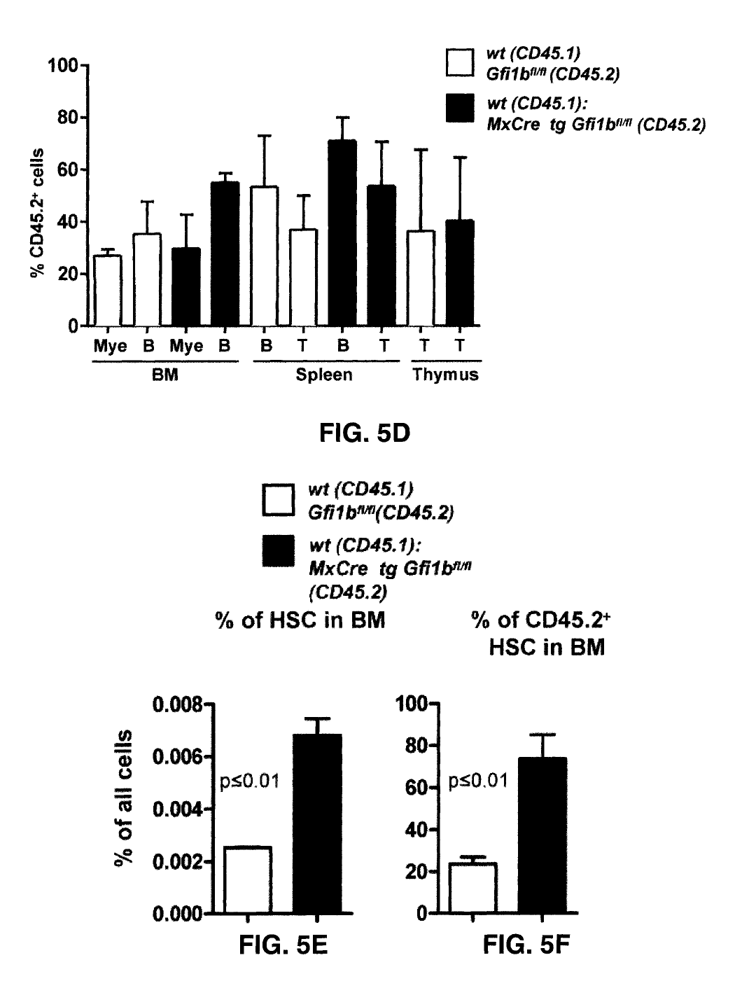

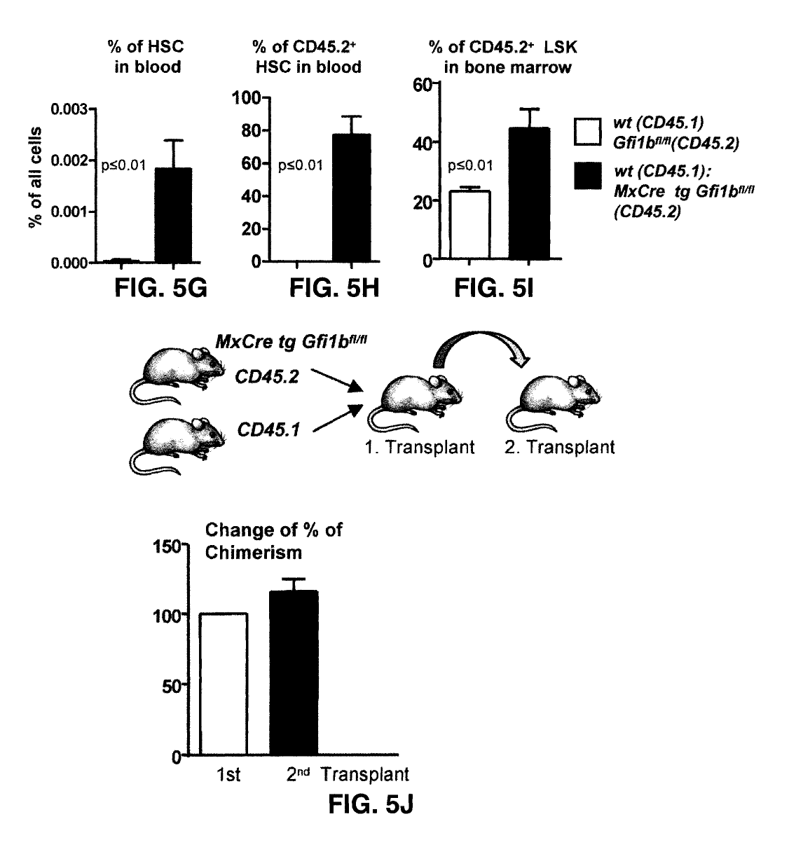

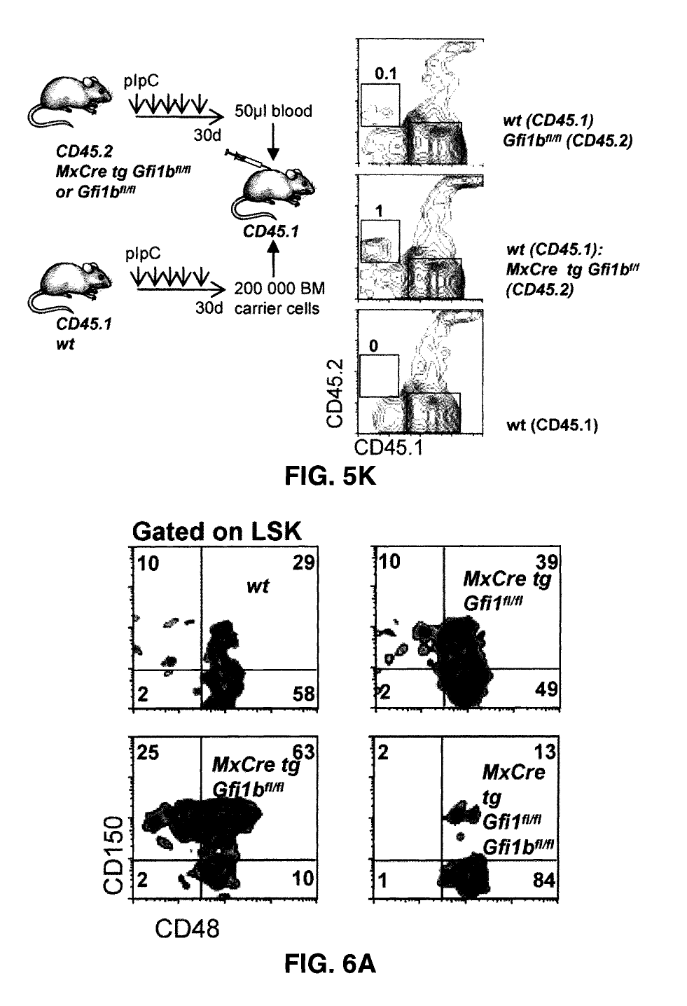

The bone marrow of Gfi1b-deficient mice contains about 39 times more phenotypically defined stem cells (HSCs, FIG. 2B, and Table 1). Yet, limiting dilution experiments suggested only 6-times more functional stem cells in Gfi1b-deficient bone marrow (Tables 3 and 4). One possible explanation for this discrepancy would be that, as a result of activation, Gfi1b^{ko/ko} HSCs are at least partially compromised in their stemness and their ability to compete with wt HSCs. To test this, a mixture of 50 sorted wt CD45.1⁺ HSCs (defined as above as LSK, CD48⁻, CD150⁺) was transplanted with either 50 sorted CD45.2⁺ wt HSCs or 50 sorted CD45.2⁺ HSCs from Gfi1b^{ko/ko} mice into syngenic recipient animals (CD45.1⁺) (FIG. 5A). It was observed that, Gfi1b^{ko/ko} HSCs could contribute to the same extent to myeloid and lymphoid lineage differentiation in blood and peripheral organs as wt CD45.2⁺ HSCs (FIGS. 5B to 5D). A significant expansion of Gfi1b-deficient CD45.2⁺ HSCs and LSK cells was again detected in the bone marrow and peripheral blood of recipient animals (FIGS. 5E to 5I). This expansion of HSCs is comparable to the result obtained after transplantation of the same number of wt and Gfi1b-deficient bone marrow cells (FIGS. 4I to L, FIGS. 5E to 5I).

It was next examined whether loss of Gfi1b might exhaust the self-renewal capacity of Gfi1b-deficient HSCs in a serial transplantation assay. Syngeneic mice (CD45.1) were transplanted with wt (CD45.1) and CD45.2⁺ Gfi1b-deficient bone marrow and the degree of chimerism in the primary and secondary recipient was determined by measuring the percentage of CD45.2⁺ cells in the blood (FIG. 5J). The experiment showed that the degree of chimerism in a secondary transplantation is maintained. The results of these experiments indicate that Gfi1b^{ko/ko} HSCs maintain their stemness and multipotency, as well as their ability to expand in blood and bone marrow beyond wt HSC numbers. It is thus unlikely that the difference between over 30-fold elevated numbers of phenotypically defined HSCs on one hand and a 6-fold elevated number of functional HSCs (limiting dilution assay) on the other hand is due to a loss of multipotency and self-renewal capacity.

HSCs residing in peripheral blood of mice have long-term potential capacity (Wright D E, et al. Science. Nov. 30 2001; 294(5548):1933-1936). Since a significant expansion of phenotypically defined HSCs was observed in the blood of Gfi1b-deficient mice, experiments to verify whether these blood HSCs represent true functional stem cells were performed. To test this, 50 μl of blood originating either from wt or Gfi1b^{ko/ko} (both CD45.2⁺) mice was transplanted alongside with 200 000 bone marrow cells from wt CD45.1 mice. Gfi1b^{ko/ko} HSCs from peripheral blood were able to give rise to CD45.2⁺ cells (FIG. 5K), indicating that Gfi1b^{ko/ko} HSCs found in blood are functionally intact stem cells. Taken together, these data indicate that Gfi1b^{ko/ko} HSCs are not compromised in their ability to compete with wt HSCs and maintain their stemness, self-renewal capacity and multi potency.

### EXAMPLE 5

### Either Gfi1b or Gfi1 Play a Role in the Maintenance of HSCs

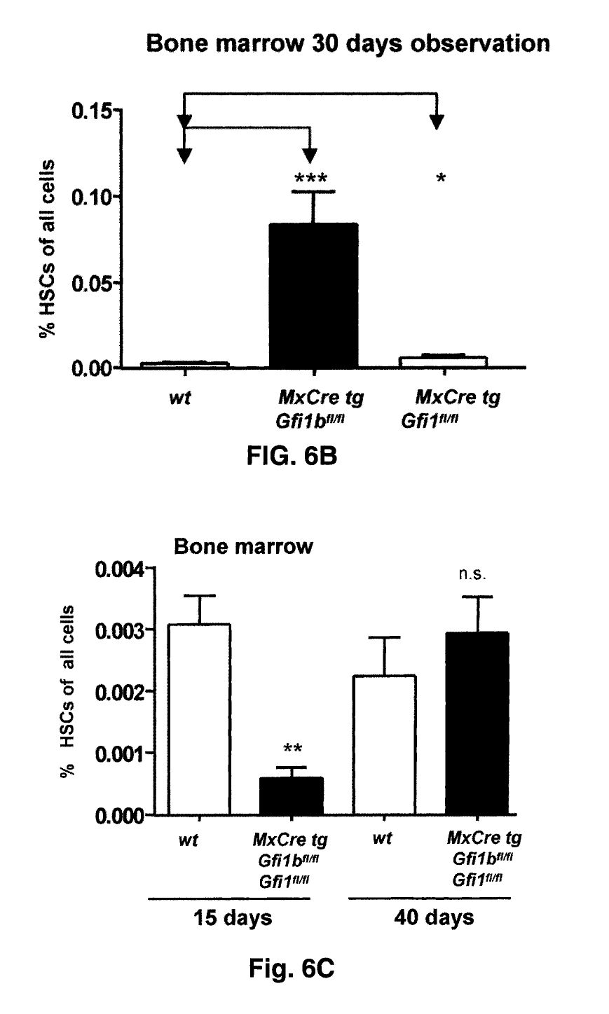

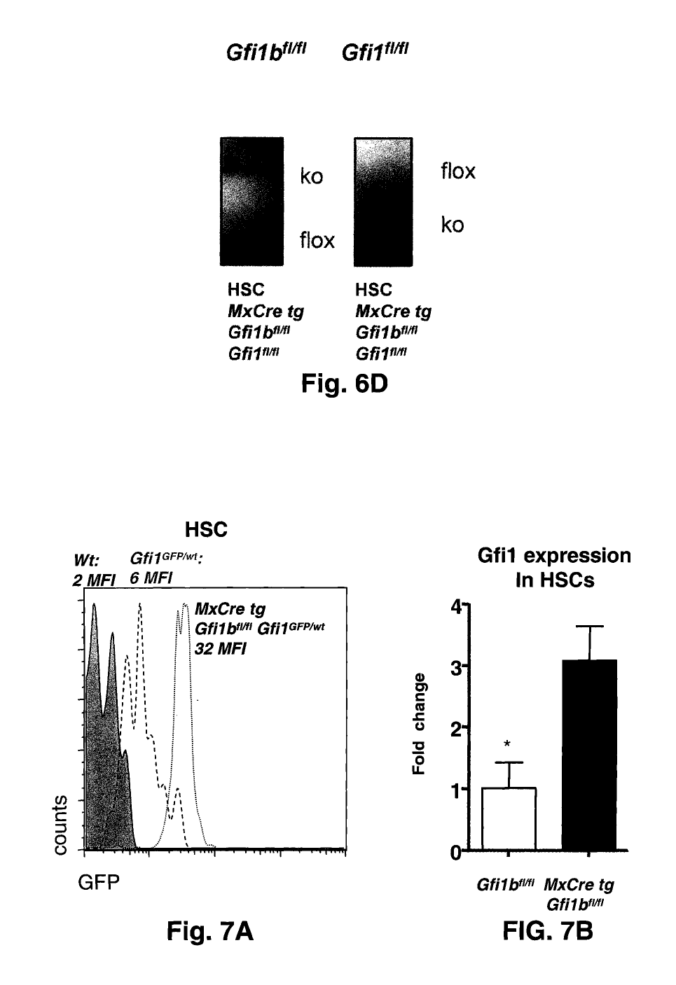

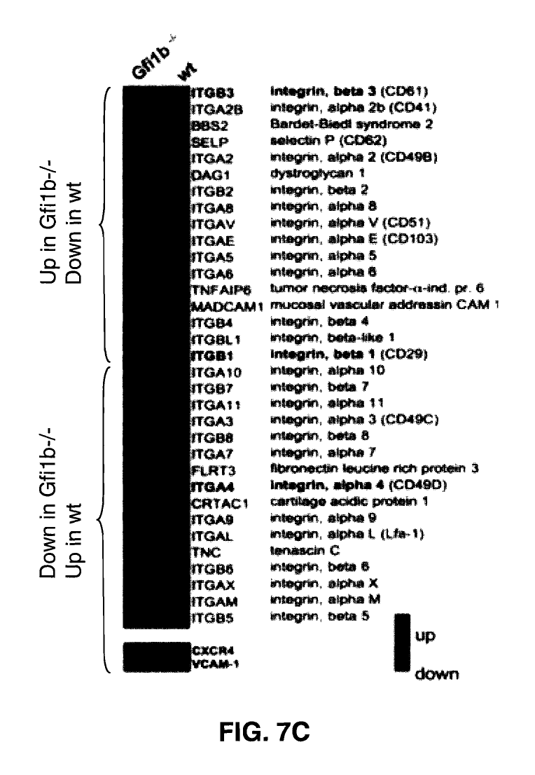

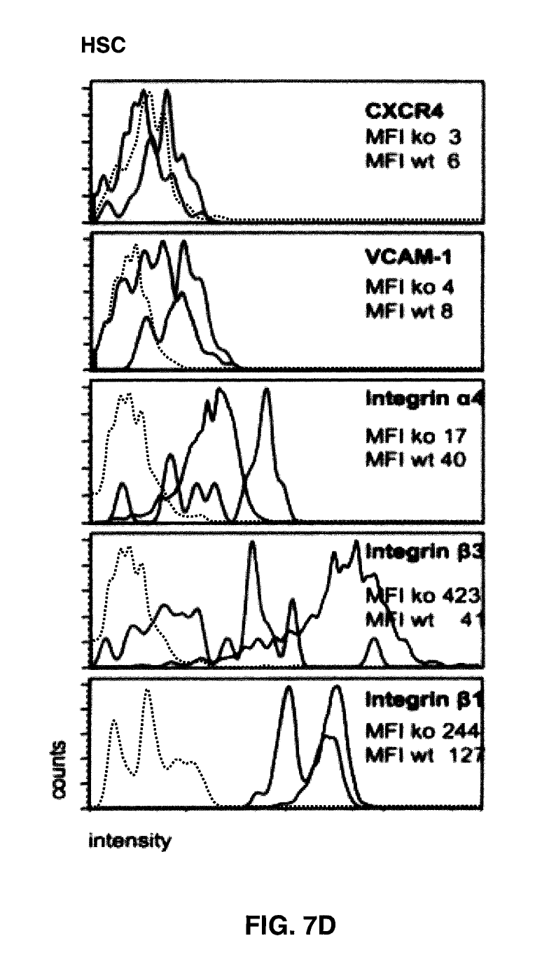

A direct comparison of both Gfi1- and Gfi1b-deficient mice confirmed that loss of Gfi1 led to an increase of HSCs, very likely due to higher cell proliferation (Zeng H et al. 2004, supra; Hock H et al. 2004, supra), but that this increase was by far not as pronounced as in Gfi1b-deficient mice (FIGS. 5A and 5B). However, when both Gfi1 and Gfi1b were deleted and mice were examined 15 days after the first plpC injection, a drastic (>5-fold) reduction of HSCs over wt numbers was observed (FIGS. 6A and 6C). Genotyping of the few HSCs remaining in these double-deficient mice showed repeatedly that one Gfi1b allele was not excised, but both Gfi1 alleles were deleted, indicating a functional Cre recombinase (FIG. 6D). It was also found that, if double Gfi1/Gfi1b-deficient mice were observed for a longer period of time (40 days after the first plpC injection), HSCs numbers were restored to wt levels (FIG. 6C), but these HSCs showed again only a partial excision of the Gfi1b locus. In addition, an upregulation of Gfi1 was measured in HSCs in which Gfi1b was deleted (FIG. 7A), and that HSCs, in which Gfi1b was deleted, up-regulated the expression of Gfi1 mRNA (FIG. 7B), showing the ability of Gfi1b and Gfi1 for crossregulation (Vassen L, et al. Nucleic Acids Res. 2005; 33(3):987-998; Doan L L, et al. Nucleic Acids Res. 2004; 32(8):2508-2519). These data demonstrate that down-regulation of Gfi1b leads to upregulation of Gfi1 in HSCs and suggest that the complete deletion of both Gfi1 and Gfi1b is incompatible with the generation or maintenance of HSCs.

### Example 6

### Loss of Gfi1b Affects Expression of Surface Molecules Important for the Hematopoietic Stem Cell Niche

To further explore how Gfi1b might function in HSCs and how its function differs from Gfi1, the relative expression levels of several genes in wt and Gfi1b^{ko/ko} HSCs was determined using Affymetrix™ gene arrays. The list of genes exhibiting at least a 2-fold difference in expression in wt vs. Gfi1b^{ko/ko} HSCs is provided in Table 6. It was found that the expression of genes encoding cell adhesion molecules and integrins was significantly deregulated in Gfi1b^{ko/ko} HSCs (FIG. 7C). Notably, the expression of VCAM-1, CXCR4 and integrin α4, which play a role in the retention of HSCs in their endosteal niche (Kiel M J et al., 2005, supra; Forsberg E C and Smith-Berdan S. Haematologica. 2009; 94:1477-1481; Wilson A et al., Curr Opin Genet Dev. 2009; 19:461-468; Kiel M J and Morrison S J. Nat Rev Immunol. 2008; 8:290-301; Martinez-Agosto J A et al., Genes Dev. 2007; 21:3044-3060; Wilson A and Trumpp A. Nat Rev Immunol. 2006; 6:93-106) were expressed at lower levels in Gfi1b^{ko/ko} HSCs as compared to wt HSCs (FIG. 7C, Table 5). On the other hand, adhesion molecules such as integrin β1 and β3 that mediate endothelial cell adhesion (Sixt M et al., Curr Opin Cell Biol. 2006; 18:482-490; Cantor J M et al., Immunol Rev. 2008; 223:236-251) were upregulated at mRNA and protein levels (FIG. 7D, Table 5), indicating that loss of Gfi1b directly or indirectly affects expression of cell surface molecules that have a role in niche organization.

TABLE 5
Change of expression of different surface proteins on stem cells after
deletion of Gfi1b.

| | Gfi1b^{fl/fl} | MxCre tg Gfi1b^{fl/fl} | |
|---|---|---|---|
| | Relative expression | Relative expression | |
| Surface protein | level | level | p-value |
| Integrin α4 | 1 | 0.48 ± 0.18 | 0.02 |
| (CD49d) | | | |
| CXCR4 | 1 | 0.53 ± 0.09 | 0.01 |
| VCAM-1 | 1 | 0.46 ± 0.07 | 0.01 |
| Integrin β3 (CD61) | 1 | 13.7 ± 1.9 | 0.02 |
| Integrin β1 (CD29) | 1 | 1.53 ± 0.2 | 0.05 |

In three independent experiments expression by Mean Fluorescence level of the different proteins was measured. To facilitate differences in up or down regulation of the different proteins, the expression in the Gfi1b^{fl/fl} was set to 1 (n=3 for all sets). Depicted are mean values and SEM. P-values are based on unpaired two-sided t-test.

TABLE 6

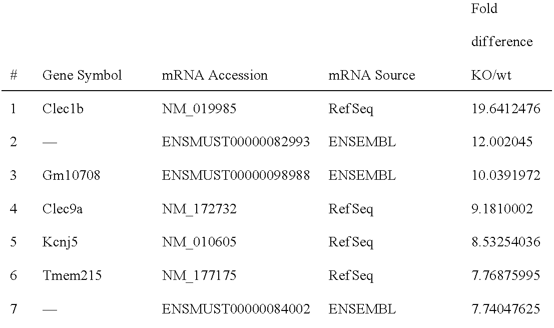

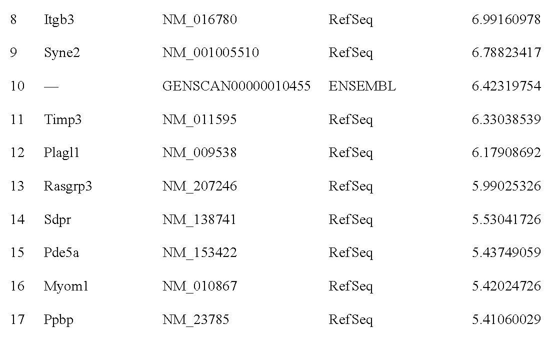

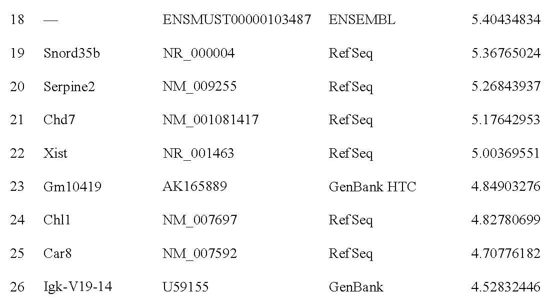

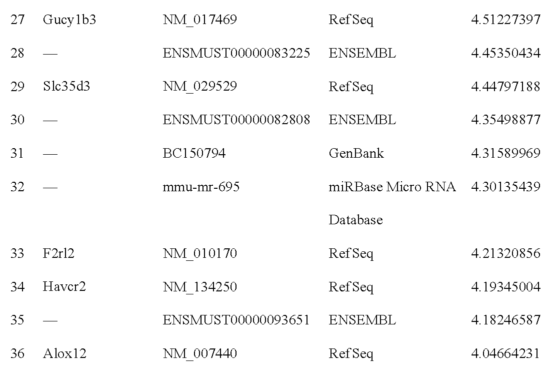

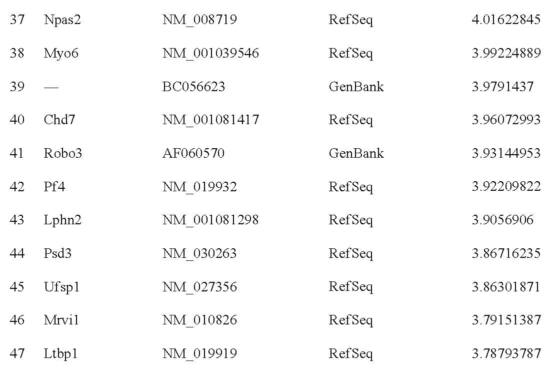

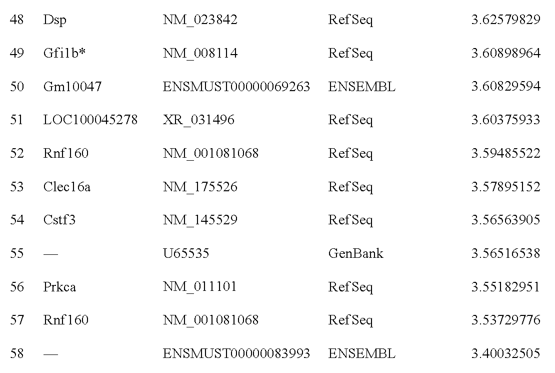

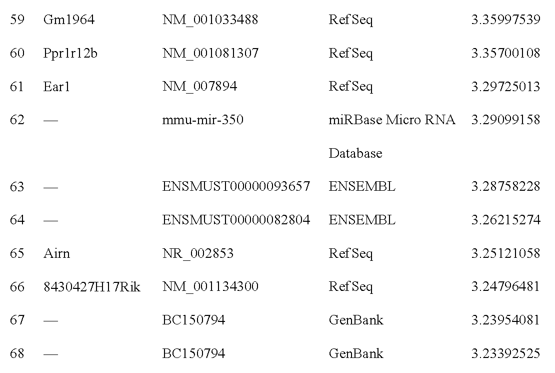

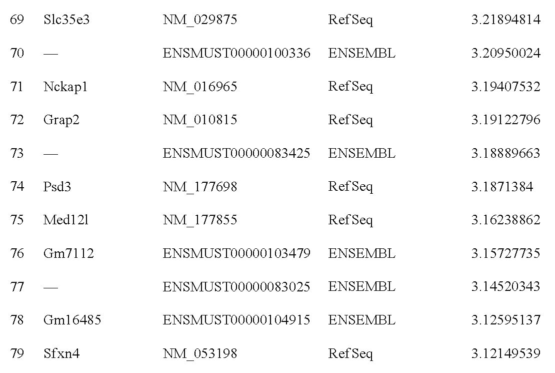

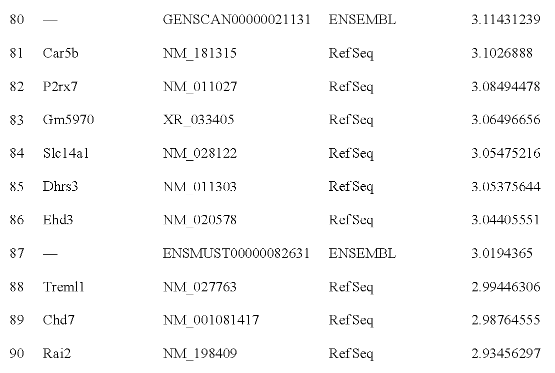

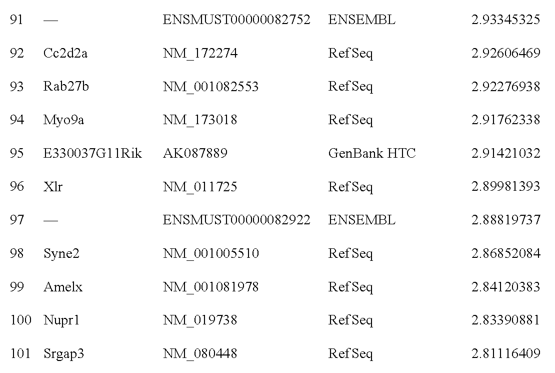

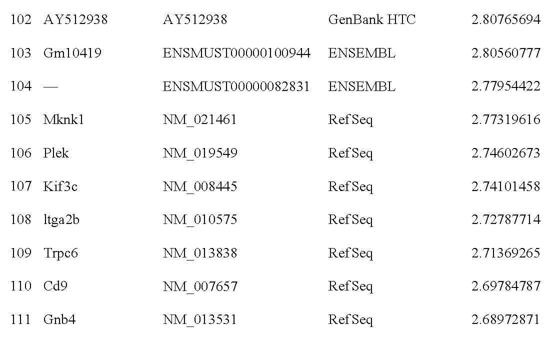

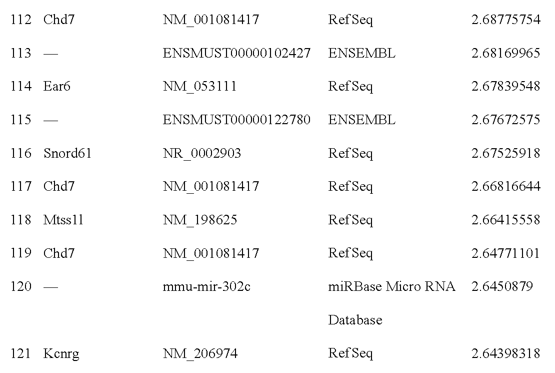

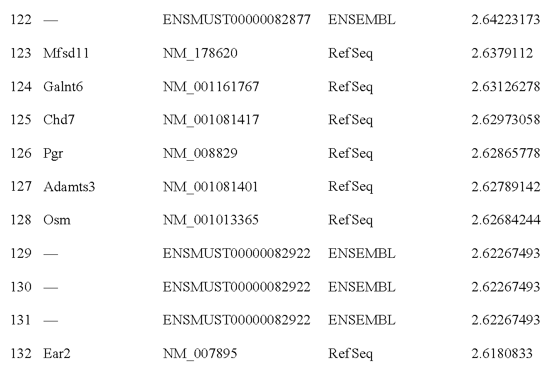

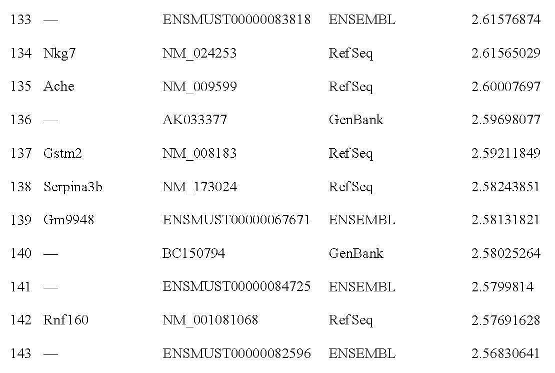

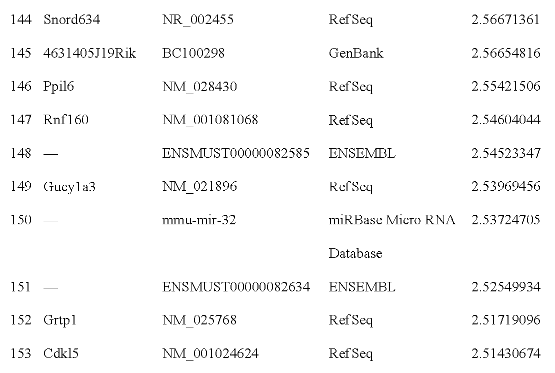

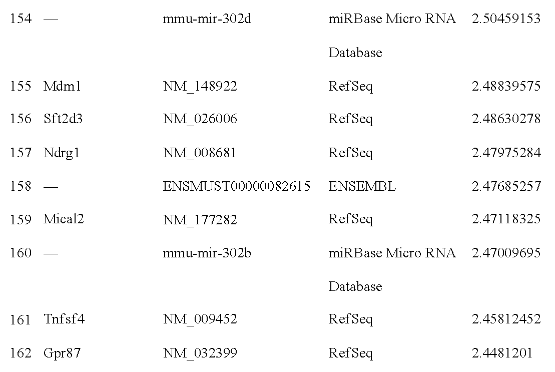

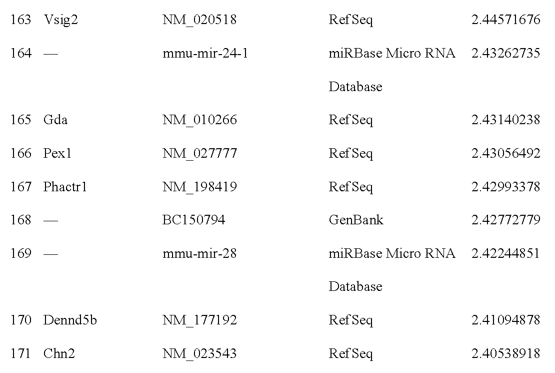

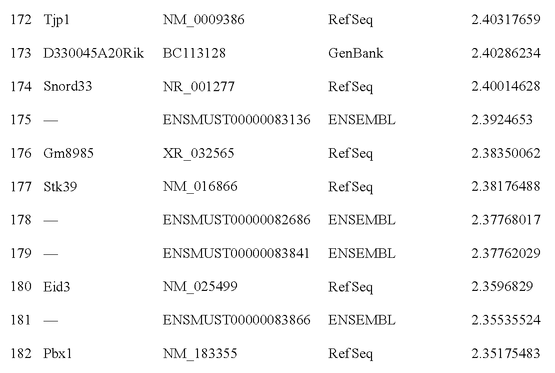

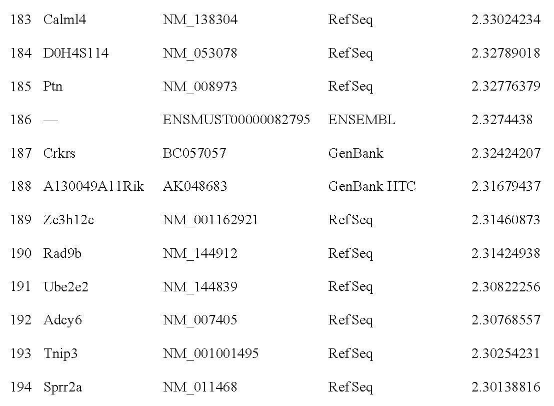

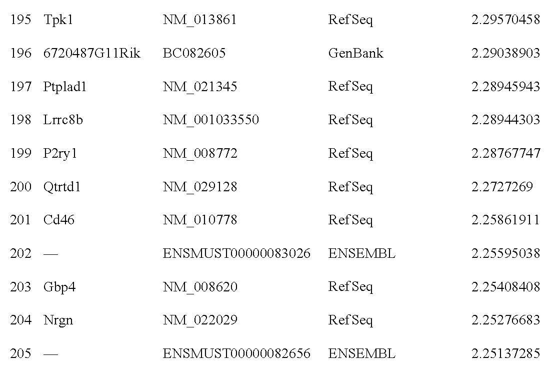

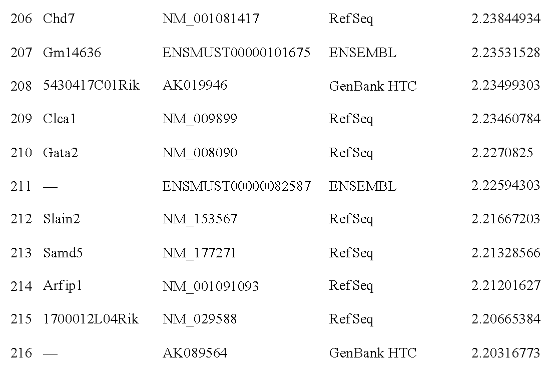

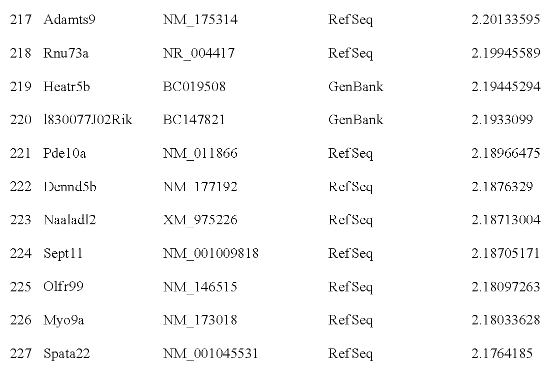

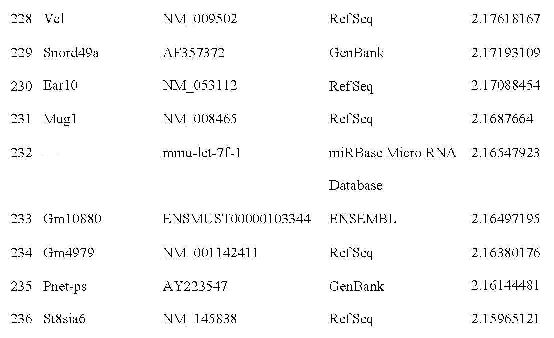

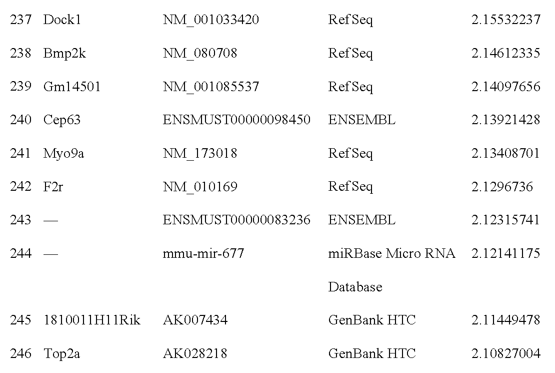

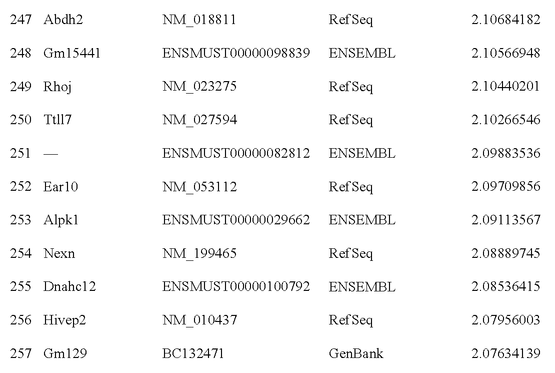

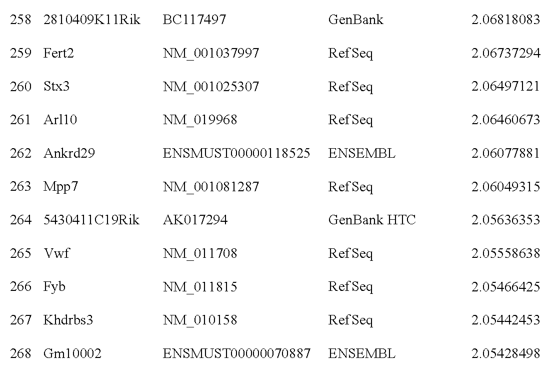

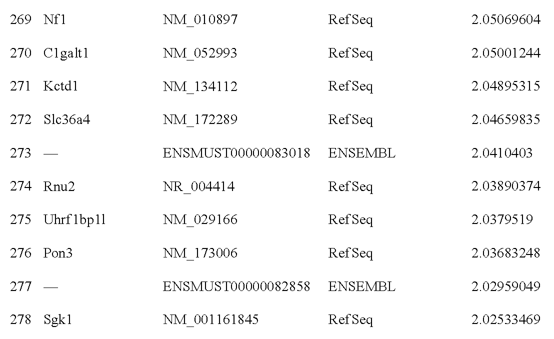

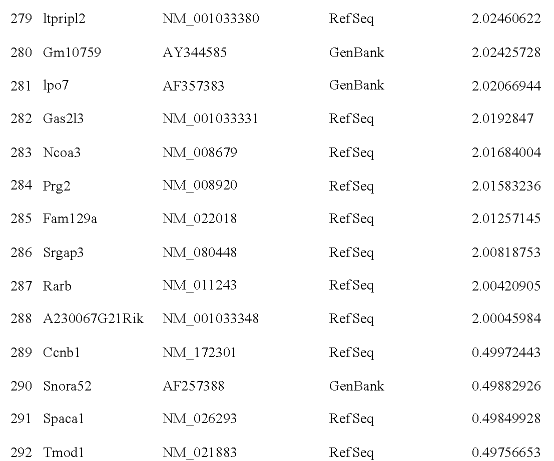

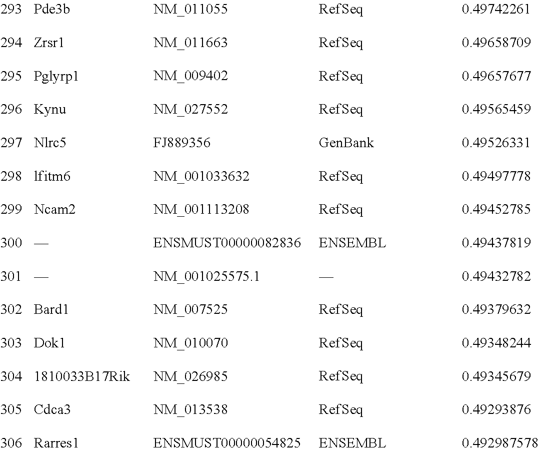

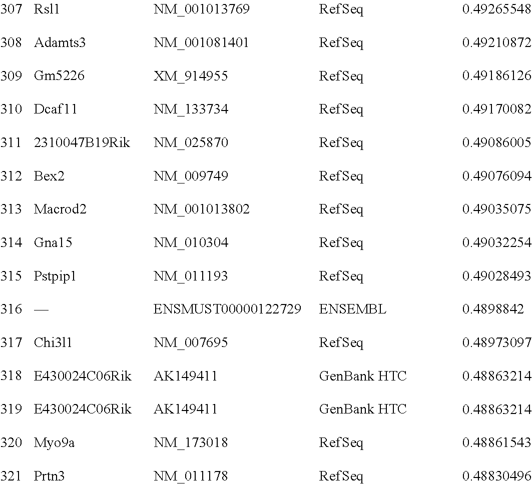

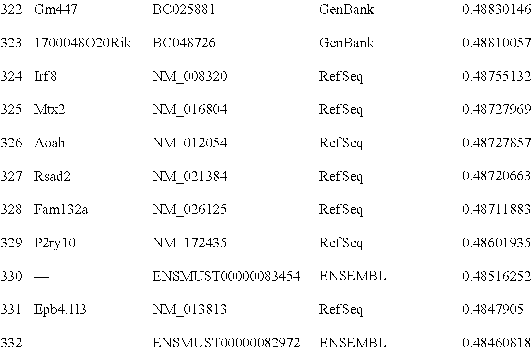

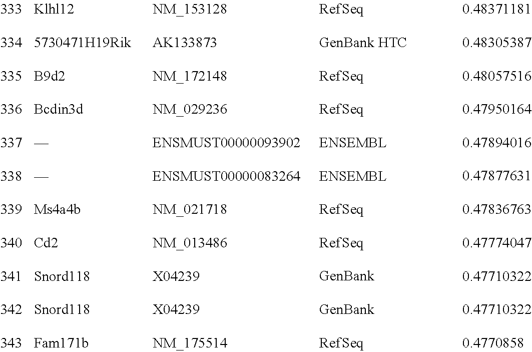

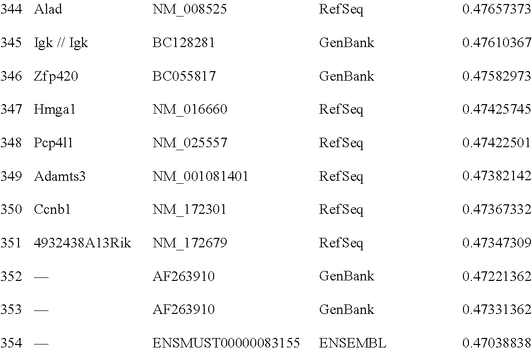

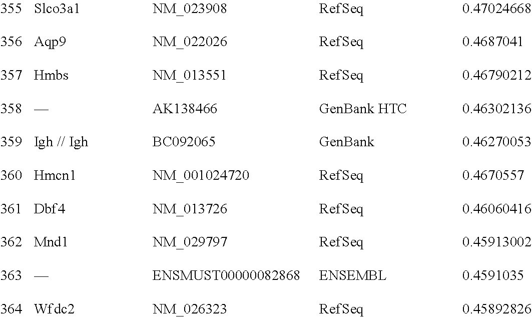

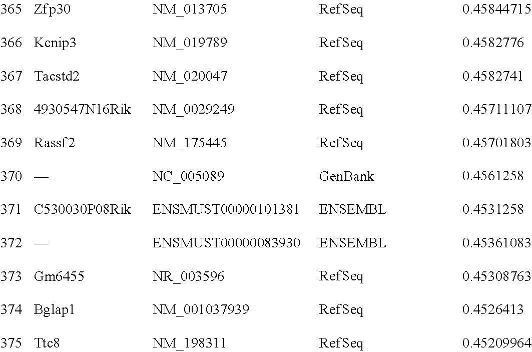

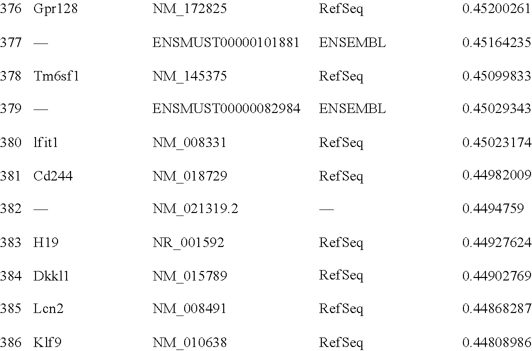

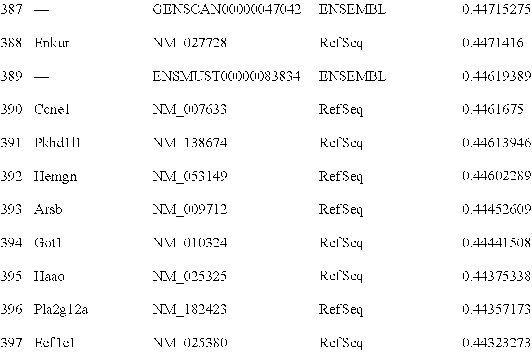

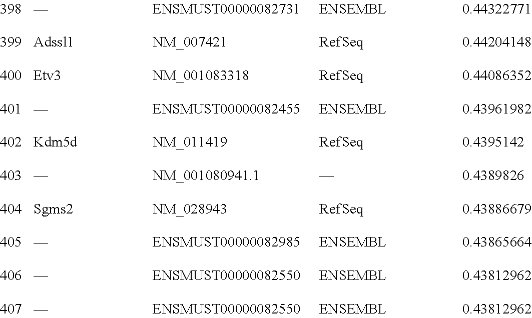

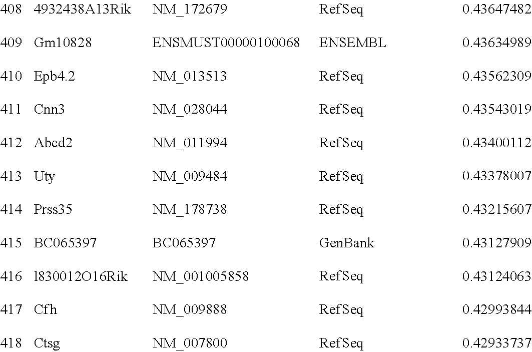

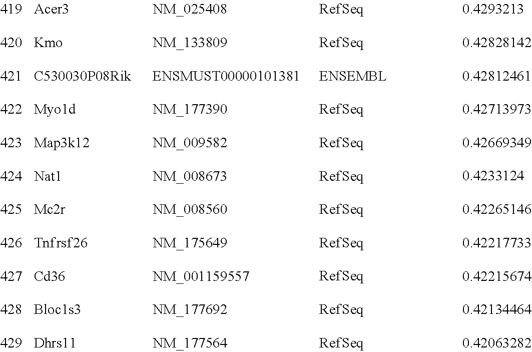

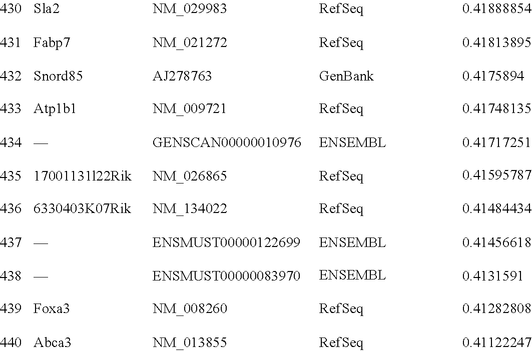

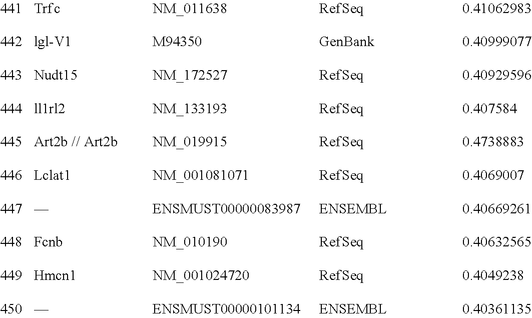

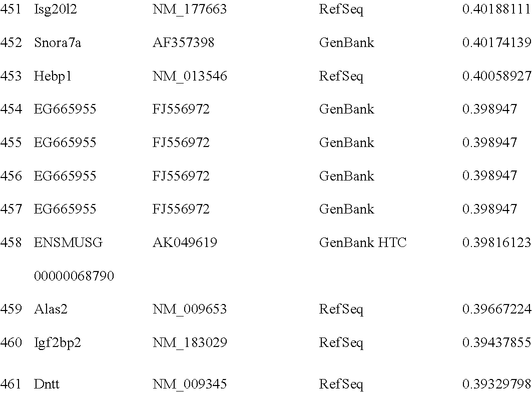

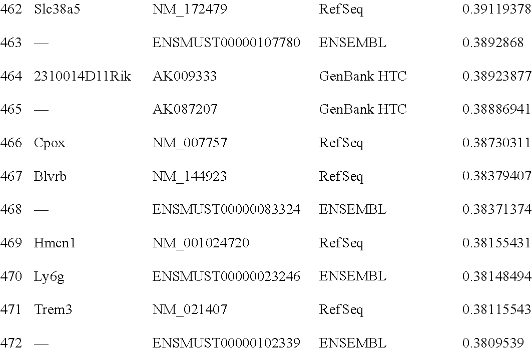

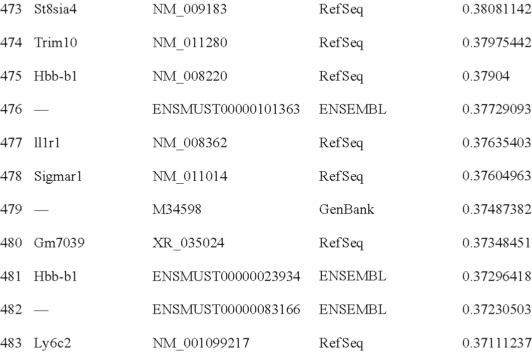

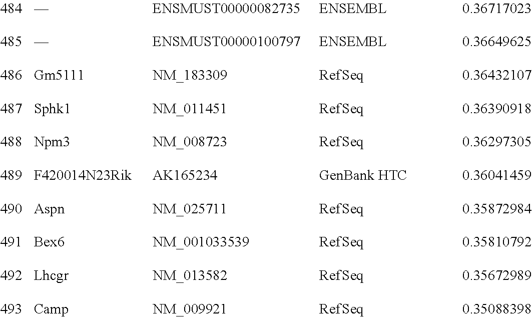

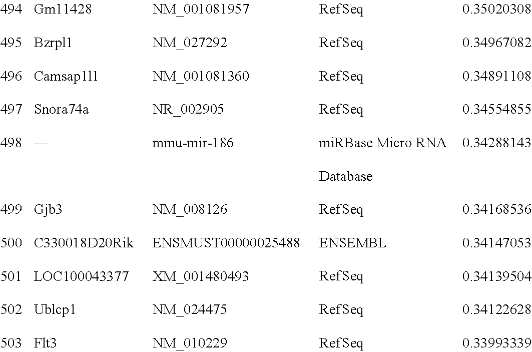

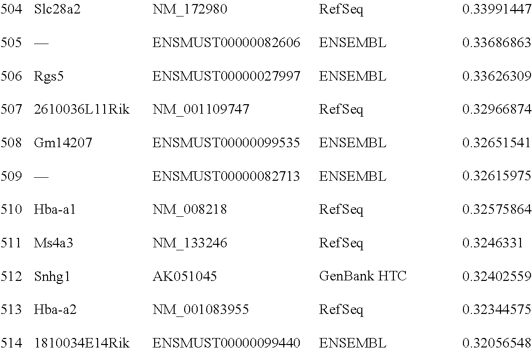

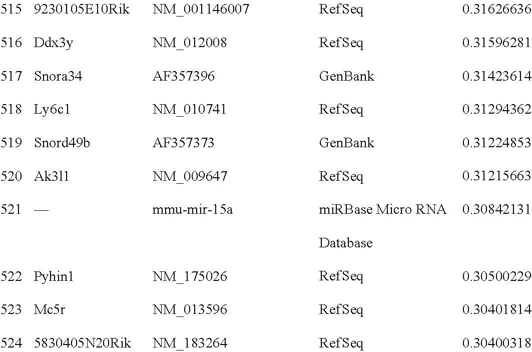

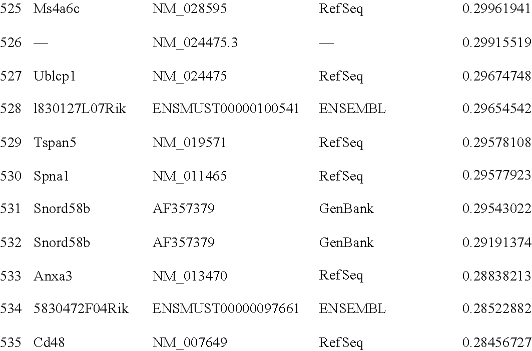

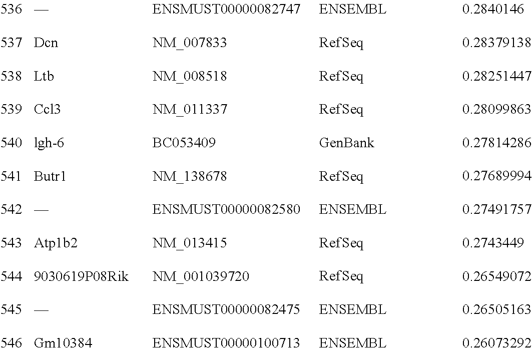

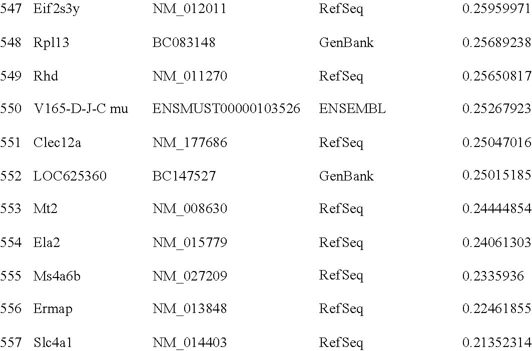

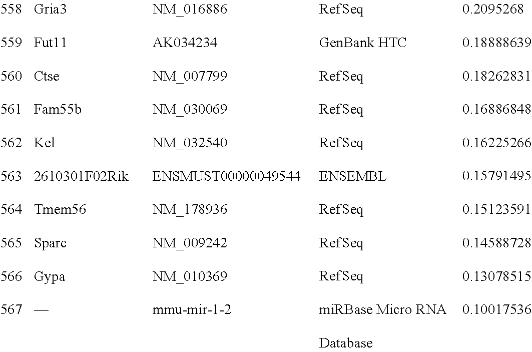

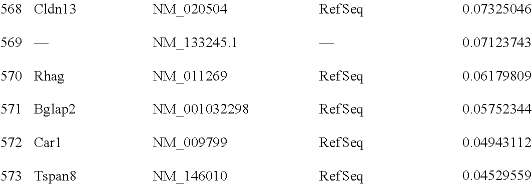

Genes exhibiting at least a 2-fold difference in expression in wt vs. Gfi1b^{ko/ko} HSCs.
Genes showing higher expression in Gfi1b^{ko/ko} HSCs are highlighted in grey.
*The apparent “higher” expression of Gfi1b mRNA in Gfi1b KO mice may be explained as follows. In the Gfi1b KO mice, those exons that are not flanked by the flox sites remain in the genome after Cre mediated deletion. Since the promoter is not deleted, a truncated Gfi1b mRNA is made, which encodes a non-functional Gfi1b protein. However, this mRNA is detected by probes ion the Affymetrix array used herein that cover sequences of the remaining exons. The level o the truncated Gfi1b mRNA is relatively up-regulated since the Gfi1b locus is under auto-regulatory control. Hence the knockout, i.e. the lack of Gfi1 protein, leads to a de-repression of the locus and the non-functional RNA is made at a higher level relative to the endogenous mRNA in non deleted cells.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

1. A method of increasing the number of hematopoietic stem cells (HSCs) in a biological system, said method comprising contacting HSCs from said biological system with an inhibitor of growth factor independence 1b (Gfi1b).

2. The method of claim 1, wherein said biological system is the bone marrow and/or blood of a subject.

3. A method of increasing the repopulation of HSCs in an HSC transplant recipient, said method comprising contacting the transplanted HSCs with an inhibitor of Gfi1b.

4. The method of claim 3, wherein said contacting occurs in a transplant donor prior to the transplantation.

5. The method of claim 3, wherein said contacting occurs in said transplant recipient after the transplantation.

6. The method of claim 1, wherein said inhibitor of Gfi1b is an inhibitory nucleic acid.

7. The method of claim 1, wherein said inhibitor of Gfi1b is a zinc-finger inhibitor.

8. The method of claim 7, wherein said zinc-finger inhibitor is Hoechst33342.

9. The method of claim 1, wherein said inhibitor of Gfi1b is a peptide comprising the amino acid sequence of SEQ ID NO: 18.

10. The method of claim 1, wherein said inhibitor of Gfi1b is an antibody recognizing an epitope within the amino acid sequence of SEQ ID NO: 18.

11-33. (canceled)

34. A method for determining whether a test compound may be useful for (i) increasing the number of hematopoietic stem cells (HSCs) in a biological system; (ii) increasing the number of HSCs in the bone marrow and/or blood of a subject; and/or (iii) increasing the repopulation of HSCs in an HSC transplant recipient, said method comprising:
(a) contacting said test compound with a Gfi1b polypeptide or a fragment thereof;
(b) determining whether said test compound binds to said Gfi1b polypeptide or fragment thereof
wherein the binding of said test compound to said Gfi1b polypeptide or fragment thereof is indicative that said test compound may be useful for (i) increasing the number of hematopoietic stem cells (HSCs) in a biological system; (ii) increasing the number of HSCs in the bone marrow and/or blood of a subject; and/or (iii) increasing the repopulation of HSCs in an HSC transplant recipient; or
(a) contacting said test compound with a cell exhibiting Gfi1b expression or activity;
(b) determining whether said test compound inhibits said Gfi1b expression or activity;
wherein the inhibition of said Gfi1b expression or activity in the presence of said test compound is indicative that said test compound may be useful for (i) increasing the number of hematopoietic stem cells (HSCs) in a biological system; (ii) increasing the number of HSCs in the bone marrow and/or blood of a subject; and/or (iii) increasing the repopulation of HSCs in an HSC transplant recipient.

35-36. (canceled)

37. A method for determining whether a test compound may be useful for (i) increasing the number of hematopoietic stem cells (HSCs) in a biological system; (ii) increasing the number of HSCs in the bone marrow and/or blood of a subject; and/or (iii) increasing the repopulation of HSCs in an HSC transplant recipient, said method comprising:
(a) contacting said test compound with a cell comprising a first nucleic acid comprising a transcriptional regulatory element comprising a Gfi1b binding sequence, operably linked to a second nucleic acid encoding a reporter protein;
(b) determining whether reporter gene expression or activity is increased in the presence of said test compound;
wherein the increase of said reporter gene expression or activity in the presence of said test compound is indicative that said test compound may be useful for (i) increasing the number of hematopoietic stem cells (HSCs) in a biological system; (ii) increasing the number of HSCs in the bone marrow and/or blood of a subject; and/or (iii) increasing the repopulation of HSCs in an HSC transplant recipient or
(a) contacting said test compound with a nucleic acid comprising a Gfi1b binding sequence in the presence of Gfi1b;
(b) determining whether said test compound inhibits the binding of Gfi1b to said nucleic acid;
wherein the inhibition of the binding of Gfi1b to said nucleic acid in the presence of said test compound is indicative that said test compound may be useful for (i) increasing the number of hematopoietic stem cells (HSCs) in a biological system; (ii) increasing the number of HSCs in the bone marrow and/or blood of a subject; and/or (iii) increasing the repopulation of HSCs in an HSC transplant recipient.

38. (canceled)

39. The method of claim 37, wherein said Gfi1b binding sequence is TAAATCAC(A/T)GCA (SEQ ID NO: 19).

40. The method of claim 37, wherein said reporter protein is luciferase.

41. The method of claim 3, wherein said inhibitor of Gfi1b is an inhibitory nucleic acid.

42. The method of claim 3, wherein said inhibitor of Gfi1b is a zinc-finger inhibitor.

43. The method of claim 42, wherein said zinc-finger inhibitor is Hoechst33342.

44. The method of claim 3, wherein said inhibitor of Gfi1b is a peptide comprising the amino acid sequence of SEQ ID NO: 18.

45. The method of claim 3, wherein said inhibitor of Gfi1b is an antibody recognizing an epitope within the amino acid sequence of SEQ ID NO: 18.


SEQUENCE LISTING (19 sequences)

SEQ ID NO: 1 - DNA, 1801 bp, Homo sapiens
  CDS (152)..(1144)
aaaaaggagaagtatctatttgtgcaaagagtcacacagttgacagagtggaggccagtcccgagagaggctttgcagttcccacctcgggaagctccggcagaacccaggcgagggacagctccggacaggtgtggggtgcacactgaaaatgccacgctccttcctggtgaagagcaagaaggctcacacctaccaccagccccgtgtgcaggaagatgaaccgctctggcctcctgcccttaccccggtgcccagagaccaggctccaagcaacagccctgtccttagcactctattcccaaaccagtgcctggactggaccaacctcaaacgagagccggagctggagcaggaccagaacttggccaggatggccccggcaccagagggccccattgtgctgtcccgaccccaggatggggactctccactgtccgactcacccccattctacaagcctagcttctcctgggacaccttggccacaacctatggccacagctaccggcaggccccctccaccatgcagtcagccttcctggagcactccgtcagcctgtacggcagtcctcttgtgcccagcactgagcccgccttggacttcagcctccgctactccccaggcatggatgcgtaccactgtgtgaagtgcaacaaggtcttctccacccctcacgggctcgaagtgcatgtgcgacgctcccatagtgggacccggcccttcgcctgtgacatctgcggcaaaaccttcggccacgctgtgagcctggagcagcacacgcacgtccactcccaggagcgcagcttcgagtgccgcatgtgcggcaaggccttcaagcgctcgtccacgctgtccacccacctgctcatccactcagacacgcggccctacccctgccagttctgcggcaagcgtttccaccagaagtccgacatgaagaagcacacctacatccacacaggtgagaagccgcacaagtgccaggtgtgcggaaaggccttcagccagagctccaacctcatcacccacagccgcaagcacacaggcttcaagcccttcagctgtgagctgtgcaccaaaggcttccagcgcaaggtggacctgcggcggcaccgcgagagccagcacaatctcaagtgaggctgcgccggctcccagctcctggccagcctgccctgcggtcctgtcacctggaggccagcctcacatgcccaaatctccagtctcctggaggtgggactggacaggagtctaccagcttgttttgagactcatgaaattgctgtgtgaccttgggcaagtcacttaccctgtctggatcaacatttctcctgctgccaagtgtgggagcctggctgggtctttctcagcagaagttgtttccaggtgtgctcaagtgccttcctctagcagagcacagaaagctagaatacccccagggagacagggatgccaagagtagaccagagctgggacccacagacagaacctccacctgcctgctgcccactgagctgggacctggtcaccttggattttagccggcctctttctggctataacaggcagagtcggagctgcctcccaccccagtcagaagcctggcaccccctctgcttcggccagatgtgctggctgactccgacttccgaccagcactcagctggcctctggggattctagctccacaaccaggccgtgaggctggagaaactggcagttattgctgtcaaaagcctgttctctcaactgctgtcaataaaattaaagatacagatttgctgccaaaaaaaaaaa

SEQ ID NO: 2 - protein, 330 aa, Homo sapiens
MPRSFLVKSKKAHTYHQPRVQEDEPLWPPALTPVPRDQAPSNSPVLSTLFPNQCLDWTNLKREPELEQDQNLARMAPAPEGPIVLSRPQDGDSPLSDSPPFYKPSFSWDTLATTYGHSYRQAPSTMQSAFLEHSVSLYGSPLVPSTEPALDFSLRYSPGMDAYHCVKCNKVFSTPHGLEVHVRRSHSGTRPFACDICGKTFGHAVSLEQHTHVHSQERSFECRMCGKAFKRSSTLSTHLLIHSDTRPYPCQFCGKRFHQKSDMKKHTYIHTGEKPHKCQVCGKAFSQSSNLITHSRKHTGFKPFSCELCTKGFQRKVDLRRHRESQHNLK

SEQ ID NO: 3 - DNA, 1663 bp, Homo sapiens
  CDS (152)..(1006)
aaaaaggagaagtatctatttgtgcaaagagtcacacagttgacagagtggaggccagtcccgagagaggctttgcagttcccacctcgggaagctccggcagaacccaggcgagggacagctccggacaggtgtggggtgcacactgaaaatgccacgctccttcctggtgaagagcaagaaggctcacacctaccaccagccccgtgtgcaggaagatgaaccgctctggcctcctgcccttaccccggtgcccagagaccaggctccaagcaacagccctgtccttagcactctattcccaaaccagtgcctggactggaccaacctcaaacgagagccggagctggagcaggaccagaacttggccaggatggccccggcaccagagggccccattgtgctgtcccgaccccaggatggggactctccactgtccgactcacccccattctacaagcctagcttctcctgggacaccttggccacaacctatggccacagctaccggcaggccccctccaccatgcagtcagccttcctggagcactccgtcagcctgtacggcagtcctcttgtgcccagcactgagcccgccttggacttcagcctccgctactccccaggcatggatgcgtaccactgtgtgaagtgcaacaaggagcgcagcttcgagtgccgcatgtgcggcaaggccttcaagcgctcgtccacgctgtccacccacctgctcatccactcagacacgcggccctacccctgccagttctgcggcaagcgtttccaccagaagtccgacatgaagaagcacacctacatccacacaggtgagaagccgcacaagtgccaggtgtgcggaaaggccttcagccagagctccaacctcatcacccacagccgcaagcacacaggcttcaagcccttcagctgtgagctgtgcaccaaaggcttccagcgcaaggtggacctgcggcggcaccgcgagagccagcacaatctcaagtgaggctgcgccggctcccagctcctggccagcctgccctgcggtcctgtcacctggaggccagcctcacatgcccaaatctccagtctcctggaggtgggactggacaggagtctaccagcttgttttgagactcatgaaattgctgtgtgaccttgggcaagtcacttaccctgtctggatcaacatttctcctgctgccaagtgtgggagcctggctgggtctttctcagcagaagttgtttccaggtgtgctcaagtgccttcctctagcagagcacagaaagctagaatacccccagggagacagggatgccaagagtagaccagagctgggacccacagacagaacctccacctgcctgctgcccactgagctgggacctggtcaccttggattttagccggcctctttctggctataacaggcagagtcggagctgcctcccaccccagtcagaagcctggcaccccctctgcttcggccagatgtgctggctgactccgacttccgaccagcactcagctggcctctggggattctagctccacaaccaggccgtgaggctggagaaactggcagttattgctgtcaaaagcctgttctctcaactgctgtcaataaaattaaagatacagatttgctgccaaaaaaaaaaa

SEQ ID NO: 4 - protein, 284 aa, Homo sapiens
MPRSFLVKSKKAHTYHQPRVQEDEPLWPPALTPVPRDQAPSNSPVLSTLFPNQCLDWTNLKREPELEQDQNLARMAPAPEGPIVLSRPQDGDSPLSDSPPFYKPSFSWDTLATTYGHSYRQAPSTMQSAFLEHSVSLYGSPLVPSTEPALDFSLRYSPGMDAYHCVKCNKERSFECRMCGKAFKRSSTLSTHLLIHSDTRPYPCQFCGKRFHQKSDMKKHTYIHTGEKPHKCQVCGKAFSQSSNLITHSRKHTGFKPFSCELCTKGFQRKVDLRRHRESQHNLK

SEQ ID NO: 5 - DNA, 1724 bp, Mus musculus
  CDS (156)..(1148)
cacagaaacgaaaaggagaagtgtctgtctgtgcagagactcataacgttgaccgagccgagagcagtccccagggacagtgtggaggttcgtggctctcgggcagaactcagaagagggacagctccctgacggtgtggcgtgcacgcagaaaaatgccacggtcctttctagtgaagagtaagaaggcacacacttaccaccagccccgggcacagggtgatgagctggtctggcctcctgctgtaattcctgtggcaaaagagcatagccagagtgccagccctcttctcagcacaccgcttccaagccagaccttggactggaacacaatcaaacaggagcgggagatgttgctgaaccagagccttcccaagatggcctcagccccagaggggcctctcgtgacaccccaaccccaggatggggaatcaccactctctgagtcaccccctttctacaagcccagcttctcctgggataccttggcctcctcctacagccacagctacacacagaccccctccaccatgcagtccgccttcctggagcgctccgtgaggctgtacggcagccccctcgtgcccagcacagagtctcccttggacttccgcctccgctactctccaggcatggacacttaccactgtgtcaagtgcaacaaggtgttctccacccctcatgggctagaagtgcatgtccgccgctctcacagcggaacccggccctttgcctgtgatgtctgtggcaaaacctttggccacgctgtgagcttggagcagcatactcacgtccactcacaggagcgaagcttcgagtgccggatgtgtggcaaagccttcaagcgttcatccaccctgtccacccacctgctcatccactcggacactcggccctacccctgccagttctgtgggaagcgcttccaccagaagtcggacatgaagaaacacacctacatccacacaggtgagaagccccacaagtgccaggtgtgtgggaaagccttcagccagagctccaacctcatcacccacagccgcaagcacacaggcttcaagccgttcagctgtgagctgtgcaccaagggcttccagcgcaaggtggacctgcgacgtcaccgtgagagtcaacacaatctcaagtgagacggttggccgcctgctttagtgtgtctcgcctgaaggccagcctctcctttccaatcctgatcccagtcctcctggaagcagcattgcccatgagcctctctgcttcttttgagactggatgacctcaaccaagccacactcctcctctgaccacagctgatgtgggagtcagttcgggcctttctgaactgaggcagtcacagacatgctctggtctcattcaatgccagagcacagacagctacaaagccctcgtggacatggcaaaaccaggcaagagccaggatccacagagaaaccctcttgcttgcttctctgggtctagtgactgaatttttagggattctctttttggctgccacagtcagagttgtcccctggcctagaggtctggcgcccctctgcttcggccagatgtgctctctgtcccccacctctcaccacatccatccgcccactgggagtcaggctcctgaggtgagccacgaggctatgaaatcagccatgatagtagaagcctgctcctatcagttgatgtaaatgtaaataaaggtgggtttatttacatgtgaa

SEQ ID NO: 6 - protein, 330 aa, Mus musculus
MPRSFLVKSKKAHTYHQPRAQGDELVWPPAVIPVAKEHSQSASPLLSTPLPSQTLDWNTIKQEREMLLNQSLPKMASAPEGPLVTPQPQDGESPLSESPPFYKPSFSWDTLASSYSHSYTQTPSTMQSAFLERSVRLYGSPLVPSTESPLDFRLRYSPGMDTYHCVKCNKVFSTPHGLEVHVRRSHSGTRPFACDVCGKTFGHAVSLEQHTHVHSQERSFECRMCGKAFKRSSTLSTHLLIHSDTRPYPCQFCGKRFHQKSDMKKHTYIHTGEKPHKCQVCGKAFSQSSNLITHSRKHTGFKPFSCELCTKGFQRKVDLRRHRESQHNLK

SEQ ID NO: 7 - DNA, 1823 bp, Mus musculus
  CDS (156)..(1247)
cacagaaacgaaaaggagaagtgtctgtctgtgcagagactcataacgttgaccgagccgagagcagtccccagggacagtgtggaggttcgtggctctcgggcagaactcagaagagggacagctccctgacggtgtggcgtgcacgcagaaaaatgccacggtcctttctagtgaagagtaagaaggcacacacttaccaccagccccgggcacagggtgatgagctggtctggcctcctgctgtaattcctgtggcaaaagagcatagccagagtgccagccctcttctcagcacaccgcttccaagccagaccttggactggaacacaatcaaacaggagcgggagatgttgctgaaccagagccttcccaagatggcctcagccccagaggggcctctcgtgacaccccaaccccaggatggggaatcaccactctctgagtcaccccctttctacaagcccagcttctcctgggataccttggcctcctcctacagccacagctacacacagaccccctccaccatgcagtccgccttcctggagcgctccgtgaggctgtacggcagccccctcgtgcccagcacagagtctcccttggacttccgcctccgctactctccaggcatggacacttaccactgtgtcaagtgcaacaaggtgttctccacccctcatgggctagaagtgcatgtccgccgctctcacagcggaacccggccctttgcctgtgatgtctgtggcaaaacctttggccacgctgtgagcttggagcagcatactcacgtccactcacagggcgtcccagccgggtccagtcctacgcccaccttggctgtcccgggccttgaggccccacctgcacctgaccccccagggcctcgtttcctccggcaggagcgaagcttcgagtgccggatgtgtggcaaagccttcaagcgttcatccaccctgtccacccacctgctcatccactcggacactcggccctacccctgccagttctgtgggaagcgcttccaccagaagtcggacatgaagaaacacacctacatccacacaggtgagaagccccacaagtgccaggtgtgtgggaaagccttcagccagagctccaacctcatcacccacagccgcaagcacacaggcttcaagccgttcagctgtgagctgtgcaccaagggcttccagcgcaaggtggacctgcgacgtcaccgtgagagtcaacacaatctcaagtgagacggttggccgcctgctttagtgtgtctcgcctgaaggccagcctctcctttccaatcctgatcccagtcctcctggaagcagcattgcccatgagcctctctgcttcttttgagactggatgacctcaaccaagccacactcctcctctgaccacagctgatgtgggagtcagttcgggcctttctgaactgaggcagtcacagacatgctctggtctcattcaatgccagagcacagacagctacaaagccctcgtggacatggcaaaaccaggcaagagccaggatccacagagaaaccctcttgcttgcttctctgggtctagtgactgaatttttagggattctctttttggctgccacagtcagagttgtcccctggcctagaggtctggcgcccctctgcttcggccagatgtgctctctgtcccccacctctcaccacatccatccgcccactgggagtcaggctcctgaggtgagccacgaggctatgaaatcagccatgatagtagaagcctgctcctatcagttgatgtaaatgtaaataaaggtgggtttatttacatgtgaa

SEQ ID NO: 8 - protein, 363 aa, Mus musculus
MPRSFLVKSKKAHTYHQPRAQGDELVWPPAVIPVAKEHSQSASPLLSTPLPSQTLDWNTIKQEREMLLNQSLPKMASAPEGPLVTPQPQDGESPLSESPPFYKPSFSWDTLASSYSHSYTQTPSTMQSAFLERSVRLYGSPLVPSTESPLDFRLRYSPGMDTYHCVKCNKVFSTPHGLEVHVRRSHSGTRPFACDVCGKTFGHAVSLEQHTHVHSQGVPAGSSPTPTLAVPGLEAPPAPDPPGPRFLRQERSFECRMCGKAFKRSSTLSTHLLIHSDTRPYPCQFCGKRFHQKSDMKKHTYIHTGEKPHKCQVCGKAFSQSSNLITHSRKHTGFKPFSCELCTKGFQRKVDLRRHRESQHNLK

SEQ ID NO: 9 - DNA, 15084 bp, Artificial Sequence
  Gfi1b conditional knock-out plasmid construct
gagctcagtgggagacagaatcctgttggttaagagtgatctgtactggcccaggatccaagagctttaaacacaggggcccctctaatgcctcagcgagaaagtgtgtgatggtggaatctaggagaggagaagcaagaggaagaagggagaaaaagaggagagaggaggaggagaggaaggagagggggaagaaaaggaagatggatggggaaagaagagaaaaggggaagagggggaagggagggaaagaaggaagggaaagaaataaaatctaaagcaaggatgagggactgtaagtcaatgctagacttttacattttaataagttagccactctctttcacccaaatgctactgaaagtttctggagaaatttccagtgggttagaagcttctgtgaagttttgataagcaaatgtggctgcactttcgcgctctggtcattggctgacgctgcagccgccagattttgacacaaataatcagattgaaaatcagggaggggaacagaaggggggcgggaaacacagaaacgaaaaggagaagtgtctgtctgtgcagagactcataacgttgaccgagccgagagcagtccccagggacagtgtggaggttcgtggctctcgggcagaactcagaagagggacagctccctgacggtgagtgctgccagacccgtgctggctgggctccgctgaaaagttcgtcctgggtgcccaaggcagggagggtgtctgcttccctttaaggtgagaggaagaatactgtggtagtctctcttctggatgcagtgctcatcaccaggggagagaagaaggaaggaacatggaccagtcccgataccccaagatagcgcaaacaccgagctttcctcctgcgaattctgtgaaacttcattgtagggggctaggcttcacccagatacacaaagtcaatcccccccttcacacacacacacacacacacacacacacacacacacacacacacacacagagcacttttctaggggcaccagaggagcccaaattggtgtccccctgggctagagctgccctgtgccaggatctcagaagggattggtagctctgatgaccctctcaaaagttcctggaagcaccggggaggggagtcagacaccctttccttgggaatttactgaagggaaatgacaaggtgaggcagaccacaggatgatggaggggaggcactgccaaaggctcttggctccttgagcatctgtagctcttctcacctagaggaaaaggtgagtttgagttgtggttttcttagaaaacagggctcattgcagagatgcccacggatggatgctgtggtctgtgactgggtcctgggaaagccagcatttttatgagactactgtggggaaagccaagtcaccctagctgtggagttggacagcctgggtactggcagagctgtgcctatccacctgtgctcttccaagtctgcaaagccctccatcaggagaggggtaacattttgatttgggggtcaacctgtagaaagtccctcggggagttgggtggctctggatgatttaggtctgatggaatcgccccagtggtgctcaccattcccagaacttcaaaaaggtgggtgcaggtcccaggtctcccttcaccggggcacagagcctgctcgcaggcagaaatagcaggagactcctccgtctgaaggcgtttgcattgaatcctttctggtgtcagtcccagccggccgtggggagtgtgggggctgtttgcccagttttgtttccattgtggtaaaacaagtccatagaactgacctttttttttttttttttttttgcttttgagttacgtggtgttcttcggcatgggcatgggacgtacactctggtcccgactccttccttctctcctttctcactacatagtaaaaaaacaacaacaacaacaacaaaaaaaaaatcaaccaaacaacaaacaaatgaacaaacaaaaacttaatttctgatccttctgcctccaccctctgtcactgtgtcctgagctacatccccagtcctttcttcttttctttcattatttttttaatctctcctttctgtttttttttttggggggggggtcgagacagggtttctctgtgtagccctggatgtcctggaactcactttgtagaccaggctggcctcgaactcagaaatccacctgcctctgcctccctggtgctgggattaaaggcgtgcgccaccatgcctggctcctttctgtttttaatttaagattactatcactaactaggcatggaggcgcatgcctttaatcctagcatttgggagccagaggcaggtagatttctgtgagtttgaggccagtctgatctacatagagagttccagaacagccagtgtctcaaaagaactacaaaagtattgttgttgttgttgtttgtttttcgtgtaagagagatgtggggctcacatgccacagcacgcatgtggaggtcagagaactactttacagtgtctgctctctttctgccccgtgggttctgggaactgaacgtaagtcatcaggcctgcatggcaagtgtctcacccactgagatagctcactgacccactttattccatttttgaccaggctgacccagaactcatgggctcaagcaatcctgacttcctcgaacttatccaggactcaagatgcttggccctaggccgagcacatcttaagagctgttcattaactatcgagaagattcatcttgttcaaccgtgtgttaatccccaaggccgtgtccttttgtgaaactgagaccccatccttattaaccaataactgttctcctgcacccagcccctgctagcctcaaatctagtcctgtgagtttgcctattctgcacaactagatatttgtccttctgtggctagcttatcttgctccctatgtttgtggggtgcatcgtgccacacatgctgaagtttccttcagttggatgtctggatcacattgtgtgctttggcctggctatattccttgctcatccatcctgcctagatggacacttgtacctctcagctcttgcagatggtgctgctgtgaacgtagacggtcaggggctctgaactctgtgctttggtgtatgcctgaaagtcatcgctcactttatttatatatttatttggtggtaatggctactgaaccctgggcttcccacctgccaggcactgactttaggttccctaaggtgacccaggctcctggccgtgactccatgacgtggccctgcagcttgtttgttgcctccctcccttctgtcttgacacataaggaagtccaaagggtcccctgtagctgaccctgagaatcagcccagtcttgcccaaccctcctgaaaagggaacctaactgaccagtgtctcagacaaggtaggtcagtccttagctgaaggtcaacagattgtacatgggattttagagtaaaggggaagtttagaaatctcttagcccaacggtctcctctgagagataggaaaaccaaacccaggttagtggtgcacgcggtcacagaatcagactcatctcccgtgggttgcacaaggttcctggagtcaggaaatcctgatgaagccactgtgctcaaaacctgggtgtgcaggatgttccagccttccaggcagctctggctgcagttccagcctgggtccattgactaagtctggatagaaggtttatgaaggatgatgacaaggggcacaaggggcctcttgctggagcccagataccatcactgtctctgttttatgaagaaatccgagtgcctttcacaatctctgccctgagctctttctcagaatggttcgtgtgaatcaaaaccgagtggttgttaattaactacattataggtaattggttcccctttctttctttcttaactcagctaacaagggccaaaagcaaactctctggatactcttggctactgctgtaggcttgggggagggggtgtgtgtctgaccaaggctcagctgtcctgatggaaaagaatgctctagaagggaaaggatagagctaaggcaacccagttggaggaagcaagacgcccaggacctcttgcttcccacgtaagcataaagcattcagtaagtgctcatgaatgtaggatggaggtcctggtcctgtgttgctctgggtttgggtcagctgacctagggtcacctctaactggaccacgggcaaaactgcctttaaggaagtctgttccaggtgaagctagttgggactctgtgtggggtctttggggatcagaggaataagaccttttgtcctcgcgcccagctgcttttgatacaatcatctataccagaggccgctgtgaggagcacagatggtgtggtggggatacagggtgagtcagaagtcaaatccaaacaggacagtagccctgggcctgcctctgtcctgacttcagagtcagtaccctctctaagccttgagatccccacctgcaaccaaggacacttcctacttccctgccccctgggacacaatgcacagagtagggcagccaccgtggcttggtttcctctttggtgtactgtgtagtgactgtctcagtgaggacctggcatgaaaggcctgggatagcctgtggcacacagaccacactgagtaactgttgtccctctgcagtgagtatggaaggcctgggcctagacatatccactccccctccctctgctcaggatttagagtccctcaagtgaagactgggggggggggcagaagggagaaacccagggagggagggggcaggagaggaaggctggcccaggaagataaggtctcggctgctatatgtaccaggatgagcaggggtccttccaaatgtcttgtttagagaactaagaatctgtcagtggaaaagatcaaagagtctaagtcccatccttcattttagagttcagagacttgaacttatgccagctctctggttcatccttaaaagtgcacctgctgggttgagcctggcctccctcctggccccttcagctcttccttagtggatggagatgccttgtgatggcttagcctaggcatctgccatgcaccttatatgctcaaggctatttcttgctcccagcattgactgacattgaatcaggccccctcttagctgatctaaatataggcttcacagcaaggagtgctggttctagctaccctgcaaacaatcccaacccctgctactccccagcggacttaaggcctctcctgcctggtttaggttcagctcctgggacagaggtgactcaagcgcccttgtcacaggctaaactctaagctgtctttcctaatcttgcctcccaccctggtctgagctgaggtcttatcgcctagccttcactgttgttcagaggcggccaaatccgttttcccgcctttgccgggcatatcacagatgtagccttcagtgagtggctggtcatgggtgggtctgggactcctcccaggctttctgagcctgaagagtctggagtgcggggaagataaaggactccacgggggttctctgtctcccagggccatgggacactgcacaggctaggacagggataggaaccattaatgggagaggaatcagatgctgttctagaggactgggcaggagacatctagcgaggtaggcatgcttcccaccttaggaactgggcagaccctttgactcatttgggcctccggttctctcccagtccctgactaagaagttaaatgttgatgcccaacaccggagttgatagacattcacctctctctgtcttcatccacacagagaccgaatgtccagatgaagtggttgggtctcctcatgtctctttccatgattaatagacggaaatgccccaccatttctggagaggggactgcgggcttaggtgctgactgcagcagctccccctgcaggacaatacaggcttcaattaagtctctacttgcctcactagcctcaagtccctttaagcacagccccaccccctaccccacccccctgctacatctgggttgtgagaacctgggatgctcaccagccttccaggctgggtagcacaccttaaagtaaaggaaattagcctggggaagtggcgagtctgcccaaatctcccccatgagcttctcttttagagagtactgttgtttagtttggtttgctttgttgggacagggtctttctatgtttgtcctggctggcctgaaacatgatctatagaccaggctggccttgaactcagggaggttctactgtccctgcctcctgagagctgggactaaaggcacgcaccaccacacctggctttttcatgagccatcaatctaaaatctaagctcagattgtgtggtgtcttgagtgtgttgaactggcctcaagaacaacttttaaaatttgttgtttgaggcagggtttctaccagtctggccctgaactcatattagctgaggatgacttcgaattgtggcttctcctgtgtccactttccaagagctggactataagaaggcaccgccataattggtcataagcttataacttcgtatagcatacattatacgaagttatcatatgcagtggagctgcaccccagcccttcaagtagcactaactggggtccacaagtgcctgcttcagggaccctagccagcccttccttctctttgccaaaacagtccaggctgactcaagtttctaagcatgaatgtactctgcctgtgactaactcaccgttcttgggggagccagatgatcaaagttaaagcgtcaataagtttggttccttctggaagttccaaaaaaatcagttcatggatctctctgagctcttggtagcaggaacctagagaggcctggctctactccctgcttttgatacacaagtcactctccctgcgcccagctttccctcttcttgtagggagggacatctggggagcaaggcctatcttatcttcaggcacatcttaacttgatgtgatattcacaggtgtcaggtggatgtgaactttggggaacgttatacaagccagcatgggatagtgtgcctttgatccaaaggactagcgcttgtcactcttagtcactcccattgcccctcctcccccgcaggtgtggcgtgcacgcagaaaaatgccacggtcctttctagtgaagagtaagaaggcacacacttaccaccagccccgggcacagggtgatgagctggtctggcctcctgctgtaattcctggtgagtctgagcttaggctatatggcccacctcatgcctgcttggcactgtcttgatgatgctgtggcatttcttctgtatctaactgtcactgaatgaatgaatggacaagtgaataaatgaatgaatgaaataaatactcatctcctctagcctttggagttgagggagcatcggactcagagcctggaaaggactatggctccagttgagatctgaagtcagggtgctctgggtttcccccacctctgcctgttctgaacagaggtgtcagcagcagcaggctggtgtccctgggtcccctctccatgatatggtaccccttgctctctgagggcattccagagcttctggtggtgactcttgggtcttgtctcctcacagtggcaaaagagcatagccagagtgccagccctcttctcagcacaccgcttccaagccagaccttggactggaacacaatcaaacaggagcgggagatgttgctgaaccagagccttcccaagatggcctcagccccaggtacccagccagggtcatcacgtcccctgcaggtgccacagaggaaagtggtagcacctacagaggctctggagtagcacttcttaggataatgatattggggagcatcagtcactggacactgtctggactctcagcatctgctggacacttctggacaggcatttgctctaaatgagggtgtttgtgtgtgtgtgtgtgtgtgtgtgtgtgtgtgtgtgtgtgtgtagcaactggggggtccatagggcagtggcctggtggtcccccaggggagggagctgaaactgcagtgaagactcacaccaatggtacacagtagggctgcatactggaaccctcaacaggaggtcaccaggagagatacagcacccgggcaacttatttcaaaattaataacctgaaccaggtgtgccggttcatgcctctaatcccagcacttgggacgcagaggcaagcagatctctgtcagttgaaggccagtctggtctacatagagagttctaggacagcaaagggtatagaaagagaccctatttagaaagaaagagagagaaagaaaggaagggaagggaaggaaagggaagaggtggagaaagagagagagagaccactggtggcagttaggaatggtcactgagcaccacaaggcagcttacagttgcctgtggctccatctggggagtccaacaccatcttctagcctctgagggcacccgcacacacacggtatacacaggtacaacatatatagagagttgaaggcaaatcgttaaaataaattaccctcacatgtctttgtacagaggggcctctcgtgacaccccaaccccaggatggggaatcaccactctctgagtcaccccctttctacaagcccagcttctcctgggataccttggcctcctcctacagccacagctacacacagaccccctccaccatgcagtccgccttcctggagcgctccgtgaggctgtacggcagccccctcgtgcccagcacagagtctcccttggacttccgcctccgctactctccaggcatggacacttaccactgtgtcaagtgcaacaaggtgagcaatgggcatgcaggccccgttcagctcagcatctattctctccaggatgctgtccctgctattatttatggctttgggtggtccttatgctgggaatcttgaggccatgctggattcagaccaagccctaactgtcttgcctttgcatcttgcctttgcttgacatgaataagttcttgggggatatagagagagcaaaacacctaccctagtccacaacaaggagatgggtcgagtaccgggccccccctcgacgaagttcctatactttctagagaataggaacttcctcgacggtatcgataagctacttagaaccacaaagtgggaatcaagagaaaaacaatgatcccacgagagatctatagatctatagatcatgagtgggaggaatgagctggcccttaatttggttttgcttgtttaaattatgatatccaactatgaaacattatcataaagcaatagtaaagagccttcagtaaagagcaggcatttatctaatcccaccccacccccacccccgtagctccaatccttccattcaaaatgtaggtactctgttctcacccttcttaacaaagtatgacaggaaaaacttccattttagtggacatctttattgtttaatagatcatcaatttctgcagacttacagcggatcgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctcccaaggcggccggagaacctgcgtgcaatccatcttgttcaatggccgatcccattatgacctgcaggtcgaaaggcccggagatgaggaagaggagaacagcgcggcagacgtgcgcttttgaagcgtgcagaatgccgggctccggaggaccttcgcgcccgccccgcccctgagcccgcccctgagcccgcccccggacccaccccttcccagcctctgagcccagaaagcgaaggagccaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaagggagccggttggcgctaccggtggatgtggaatgtgtgcgaggccagaggccacttgtgtagcgccaagtgccagcggggctgctaaagcgcatgctccagactgccttgggaaaagcgcctcccctacccggtagaattggccgcgaagttcctatactttctagagaataggaacttcggccgccaccgcggtgagagcttataacttcgtatagcatacattatacgaagttattctagagaattcctgcagcccatcagaaacagcatctacgtggtcctagatagctttggcattctttttttttttttttttttttttttttttgagacaaggtctcatgtagctcaggctggacctgagctctctttgtagccaaggataaccctgaactctttatctttctccctccacctcctcaagtattcaccaccacagctagttttgcctgctttaatgcagtactagggcctcatgtgtaatatacaagcgctctatcaactgagctacctctcagtcccctagccctgagatcttacccatgggttattcgtcctccagatgttcatggaggagccttctggttccctcccacagccccattttatagataggaaactgccacagagaggtgagcgactccctgggatgtgcagcagaacagccggaccgcctgaggtgcccttctcctgctctatcctgctctggagttaggcccatggccagggtgaccacaggcctgcctccttccagggacctgactctttgctctgccccaggtgttctccacccctcatgggctagaagtgcatgtccgccgctctcacagcggaacccggccctttgcctgtgatgtctgtggcaaaacctttggccacgctgtgagcttggagcagcatactcacgtccactcacaggtgagtaactgacaaaggctgaaggggacaaaggtgggggaggagcaaaggtggaggaggagcaaaggtggaggaggagcaaaggtgggggaggagcaaaggtgggggaggagcaaaggtgggggaggagtaaaggtgggggaggagcagactccctgacttgtctccctgggtctgagtcatctaacatactgggtctctgattggctgaaatggtttcacatctagttggaaggaactggggatttaggggtgggagctgggttatgtcagtgagaagccatcctatgctctgtgtggccccgcaggcatcctcaattcttagagttttagagttcaatttaaatacacgtcctccctcccacccccaccccatctctgtttgggacccattcctttccagagcagaacaggtaactaaccagcactctgtctgggtcacacaggccggttaccaacacctggggaggtgggaaaggtggtgggaggtgaccctcgaaccttccgtccctcggcaacaggctggaagctggctctgtcttctacttcgaagcctcccacagaccaaccaggccaggctagaactgccccagtgcacagatgaggaaaacaatgggcttgccccaagcatacggctgctgggctgctgggagtagggtctcagctggtagagtgcttgcctagtacacagaagccctaacaccagatgaaccaggaatggcaggcctagaacagcactcaggaggtggaagctcaaagttatcactgcctacataataagttcaaggccaaactgggctacccaagaacctatcctaactaaattgagtcagcatgtcactagctaagaggaactcactctgcgttactggtgaatccctgaaaaacttagttgcttcatcaatcagtcaccaaacacaccctgaggcttattttgaaaagtatactgtgaaaggcacagggagagatttcagaggaagcaaatcaggcccaaagcctcagtcctcagttgaggaagcaggaatggggcggggttcttcctgcttctctggacccctgttctcgaggccaccccaacggagtgtcctctttctcagggcgtcccagccgggtccagtcctacgcccaccttggctgtcccgggccttgaggccccacctgcacctgaccccccagggcctcgtttcctccggcaggagcgaagcttcgagtgccggatgtgtggcaaagccttcaagcgttcatccaccctgtccacccacctgctcatccactcggacactcggccctacccctgccagttctgtgggaagcgcttccaccagaagtcggacatgaagaaacacacctacatccacacaggtgagcacccaggccgagttcgagggaaggccataggatcgcctgcctggcttcccccatcatttgccaagaggcccccttgaggagggtctggagggctgcacaatcactcaaagtaaagaaacactggcaggaaagccaagccaacaaaggcctgagggcagaaggccagcgtgggcgtggattctccaccccctgtcgctcactctgtccactctacagagtgagcggccttgcgcagccaaggaatcaaggaagggccagcctgtgccagcttggaccagctgagaaagccagaaatctagagaaagactgtaccagtaacctgacacattatggtgacatagatgcctgacaaaatgtcactgggcctcacagtttgagacggagtccatcacggcaggactgctcagtggtaggagcatgaggcagctggtcacactgctttaggaaggagagggcggaatgctggtgtgcagcttgctctcccccttttattctgtcagtcccagcccatgggacagtgccagctgccatatttaggatgaaccttcagttagcccccgccccccccaaattcctcacagacattcccagcagtgtgtctcctgggtgacctaagtcccatcaagtagacagtgttgcccatcagaaaagcattttgcttgtttgtgtgcttgcttgcttattgagacaaatactcaggtagctgaggctagccttggagttaatatgtagttgaggctgacctcaacctctaacttgcaatttctctccctcaggatgagagggttacagatgtatgcatttccttcctttcttcctccccccttccttccctcccccccttccttccctcccttcctcccttccttccttccttccttttttgttgttgttgttttgttctgttttggtttggtttagggttatttgttatttttttttctttttgtttttgggggatagggtttctctgtgtgtaggcctggctgtcctggaactcactctataaatcaagctggccttgaactcagagatccatctgcctctgtctcccaagggctgggatcaaaggtgtggccatcacacttaactactttccaccattttctaaggttggccataaaaacccagtagcgaatgtgtgacagggtggtggctgcacgcctagcactgccacctgaacccacctgcggtttgtcccttccccaggtgagaagccccacaagtgccaggtgtgtgggaaagccttcagccagagctccaacctcatcacccacagccgcaagcacacaggcttcaagccgttcagctgtgagctgtgcaccaagggcttccagcgcaaggtggacctgcgacgtcaccgtgagagtcaacacaatctcaagtgagacggttggccgcctgctttagtgtgtctcgcctgaaggccagcctctcctttccaatcctgatcccagtcctcctggaagcagcattgcccatgagcctctctgcttcttttgagactggatgacctcaaccaagccacactcctcctctgaccacagctgatgtgggagtcagttcgggcctttctgaactgaggcagtcacagacatgctctggtctcattcaatgccagagcacagacagctacaaagccctcgtggacatggcaaaaccaggcaagagccaggatccacagagaaaccctcttgcttgcttctctgggtctagtgactgaatttttagggattctctttttggctgccacagtcagagttgtcccctggcctagaggtctggcgcccctctgcttcggccagatgtgctctctgtcccccacctctcaccacatccatccgcccactgggagtcaggctcctgaggtgagccacgaggctatgaaatcagccatgatagtagaagcctgctcctatcagttgatgtaaatgtaaataaaggtgggtttatttacatgtgaccttgtttgtaaaactcctactaggtccccaggactgcctgaggacttgagggccttgacagggagctaacagagagaagcaccatgacaggtgtccag

SEQ ID NO: 10 - DNA, 27 bp, Artificial Sequence
  Synthetic oligonucleotide
ggtttctaccagtctggccctgaactc

SEQ ID NO: 11 - DNA, 27 bp, Artificial Sequence
  Synthetic oligonucleotide
ctcacctctctgtggcagtttcctatc

SEQ ID NO: 12 - DNA, 26 bp, Artificial Sequence
  Synthetic oligonucleotide
tacattcatgcttagaaacttgagtc

SEQ ID NO: 13 - RNA, 19 nt, Artificial Sequence
  TRIAD1 siRNA
uugugaggaagaggaagaa

SEQ ID NO: 14 - RNA, 21 nt, Artificial Sequence
  TRIAD1 siRNA
aauugugaggaagaggaagaa

SEQ ID NO: 15 - DNA, 45 bp, Artificial Sequence
  GFI1B shRNA
gcctagcttctcctgggacttcaagagagtcccaggagaagctag

SEQ ID NO: 16 - DNA, 21 bp, Artificial Sequence
  GFI1B shRNA
cccattctacaagcctagctt

SEQ ID NO: 17 - DNA, 21 bp, Artificial Sequence
  GFI1B shRNA
ccttagcactctattcccaaa

SEQ ID NO: 18 - protein, 20 aa, Homo sapiens
MPRSFLVKSKKAHTYHQPRV

SEQ ID NO: 19 - DNA, 12 bp, Homo sapiens
taaatcacwgca